(19) United States
(12) Patent Application Publication
(10) Pub. No.: US 2003/0236212 A1
(43) Pub. Date: Dec. 25, 2003

(54) Functionalized long chain derivatives as acyl coenzyme-A mimics, compositions thereof, and methods of cholesterol management and related uses

(76) Inventors: Jean-Louis Dasseux, Ann Arbor, MI (US); Carmen Daniela Oniciu, Ann Arbor, MI (US)

Correspondence Address: PENNIE & EDMONDS LLP, 1667 K STREET NW, SUITE 1000, WASHINGTON, DC, 20006

(21) Appl. No.: 10/410,262

(22) Filed: Apr. 10, 2003

Related U.S. Application Data
(60) Provisional application No. 60/371,511, filed on Apr. 10, 2002.

(51) Int. Cl. A61K 48/00; A61K 31/7076; A61K 31/7072; C07H 21/02; C07H 21/04; C07H 19/04; A61K 31/66; C07F 9/02

(52) U.S. Cl. 514/44; 514/47; 514/48; 514/50; 514/125; 536/23.1; 536/26.1; 558/178; 514/114

(57) ABSTRACT

The invention relates to novel Acyl coenzyme-A mimics, compositions comprising ketone compounds, and methods useful for treating and preventing cardiovascular diseases, dyslipidemias, dysproteinemias, and glucose metabolism disorders comprising administering a composition comprising a ketone compound. The Acyl coenzyme-A mimics, compositions, and methods of the invention are also useful for treating and preventing Alzheimer's Disease, Syndrome X, peroxisome proliferator activated receptor-related disorders, septicemia, thrombotic disorders, obesity, pancreatitis, hypertension, renal disease, cancer, inflammation, bacterial infection and impotence. In certain embodiments, the Acyl coenzyme-A mimics, compositions, and methods of the invention are useful in combination therapy with other therapeutics, such as hypocholesterolemic and hypoglycemic agents.

This application claims priority to U.S. provisional application No. 60/371,511, filed Apr. 10, 2002, the entirety of which is incorporated herein by reference.

### 1. FIELD OF THE INVENTION

The invention relates to acyl-Coenzyme-A mimics; compositions comprising an acyl coenzyme-A mimic; and methods for treating or preventing a disease or disorder, such as cardiovascular disease, dyslipidemia, dyslipoproteinemia, a disorder of glucose metabolism, Alzheimer's Disease, Syndrome X, a peroxisome proliferator activated receptor-associated disorder, septicemia, a thrombotic disorder, obesity, pancreatitis, hypertension, renal disease, cancer, inflammation, bacterial infection and impotence, comprising the administration of an acyl coenzyme-A mimic.

### 2. BACKGROUND OF THE INVENTION

Obesity, hyperlipidemia, and diabetes have been shown to play a casual role in atherosclerotic cardiovascular diseases, which currently account for a considerable proportion of morbidity in Western society. Further, one human disease, termed “Syndrome X” or “Metabolic Syndrome”, is manifested by defective glucose metabolism (insulin resistance), elevated blood pressure (hypertension), and a blood lipid imbalance (dyslipidemia). See e.g. Reaven, 1993, Annu. Rev. Med. 44:121-131.

The evidence linking elevated serum cholesterol to coronary heart disease is overwhelming. Circulating cholesterol is carried by plasma lipoproteins, which are particles of complex lipid and protein composition that transport lipids in the blood. Low density lipoprotein (LDL) and high density lipoprotein (HDL) are the major cholesterol-carrier proteins. LDL are believed to be responsible for the delivery of cholesterol from the liver, where it is synthesized or obtained from dietary sources, to extrahepatic tissues in the body. The term “reverse cholesterol transport” describes the transport of cholesterol from extrahepatic tissues to the liver, where it is catabolized and eliminated. It is believed that plasma HDL particles play a major role in the reverse transport process, acting as scavengers of tissue cholesterol. HDL is also responsible for the removal non-cholesterol lipid, oxidized cholesterol and other oxidized products from the bloodstream.

Atherosclerosis, for example, is a slowly progressive disease characterized by the accumulation of cholesterol within the arterial wall. Compelling evidence supports the belief that lipids deposited in atherosclerotic lesions are derived primarily from plasma apolipoprotein B (apo B)-containing lipoproteins, which include chylomicrons, CLDL, IDL and LDL. The apo B-containing lipoprotein, and in particular LDL, has popularly become known as the “bad” cholesterol. In contrast, HDL serum levels correlate inversely with coronary heart disease. Indeed, high serum levels of HDL is regarded as a negative risk factor. It is hypothesized that high levels of plasma HDL is not only protective against coronary artery disease, but may actually induce regression of atherosclerotic plaque (e.g., see Badimon et al., 1992, Circulation 86:(Suppl. III)86-94; Dansky and Fisher, 1999, Circulation 100: 1762-3.). Thus, HDL has popularly become known as the “good” cholesterol.

### 2.1 Fatty Acid Synthesis

The first step in fatty acid synthesis is the carboxylation of acetyl coenzyme A (coA) to malonyl coA, a process catalyzed by the enzyme acetyl coA carboxylase. Malonyl coA, as well as acetyl coA, are linked to an acyl carrier protein (ACP), producing malonyl-ACP and acetyl-ACP, respectively. Malonyl-ACP and acetyl-ACP condense to form acetoactyl ACP and, following a series of reactions, butryl-ACP is formed. Fatty acid elongation proceeds by sequential addition of malonyl coA subunits (by condensation of malonyl-ACP) to butryl-ACP, and is catalyzed by an enzyme system referred to as fatty acid synthase, which in eukaryotic cells is part of a multienzyme complex. See generally Stryer, 1988, Biochemistry W. H. Freeman & Co., New York, at chapter 20.

Fatty acid synthases, also known as fatty acid ligases, are classified on the basis of the length of the carbon chain of the fatty acid to which they conjugate acetyl coA (in the form of a malonyl-ACP). Acetate-CoA ligase (EC 6.2.1.1, also known as acetyl-CoA synthetase and short chain fatty acyl-CoA synthetase) activates C2-C4 fatty acids, the butyrate-CoA ligase (EC 6.2.1.2, also known as medium chain acyl-CoA synthetase and propionoyl-CoA synthetase) activates C4-C12 while the long-chain fatty acid-CoA ligase (EC 6.2.1.3, also known as palmitoyl-CoA synthetase and long-chain acyl CoA synthetase) activates long-chain fatty acids C10-C22. Novel fatty acid syntheses are being actively identified. For example, Steinberg et al. have recently identified a human very long-chain fatty acid ligase homologous to the Drosophila “bubblegum” protein (Steinberg et al, 2000, J. Biol. Chem. 275:35162-69), and Fujino et al. have identified two murine medium-chain fatty acid ligases called MACS1 and Sa (Fujino et al., 2001, J. Biol. Chem. 276:35961-66).

### 2.2. Cholesterol Transport

The fat-transport system can be divided into two pathways: an exogenous one for cholesterol and triglycerides absorbed from the intestine and an endogenous one for cholesterol and triglycerides entering the bloodstream from the liver and other non-hepatic tissue.

In the exogenous pathway, dietary fats are packaged into lipoprotein particles called chylomicrons, which enter the bloodstream and deliver their triglycerides to adipose tissue for storage and to muscle for oxidation to supply energy. The remnant of the chylomicron, which contains cholesteryl esters, is removed from the circulation by a specific receptor found only on liver cells. This cholesterol then becomes available again for cellular metabolism or for recycling to extrahepatic tissues as plasma lipoproteins.

In the endogenous pathway, the liver secretes a large, very-low-density lipoprotein particle (VLDL) into the bloodstream. The core of VLDL consists mostly of triglycerides synthesized in the liver, with a smaller amount of cholesteryl esters either synthesized in the liver or recycled from chylomicrons. Two predominant proteins are displayed on the surface of VLDL, apolipoprotein B-100 (apo B-100) and apolipoprotein E (apo E), although other apolipoproteins are present, such as apolipoprotein CIII (apo CIII) and apolipoprotein CII (apo CII). When a VLDL reaches the capillaries of adipose tissue or of muscle, its triglyceride is extracted. This results in the formation of a new kind of particle called intermediate-density lipoprotein (IDL) or VLDL remnant, decreased in size and enriched in cholesteryl esters relative to a VLDL, but retaining its two apoproteins.

In human beings, about half of the IDL particles are removed from the circulation quickly, generally within two to six hours of their formation. This is because IDL particles bind tightly to liver cells, which extract IDL cholesterol to make new VLDL and bile acids. The IDL not taken up by the liver is catabolized by the hepatic lipase, an enzyme bound to the proteoglycan on liver cells. Apo E dissociates from IDL as it is transformed to LDL. Apo B-100 is the sole protein of LDL.

Primarily, the liver takes up and degrades circulating cholesterol to bile acids, which are the end products of cholesterol metabolism. The uptake of cholesterol-containing particles is mediated by LDL receptors, which are present in high concentrations on hepatocytes. The LDL receptor binds both apo E and apo B-100 and is responsible for binding and removing both IDL and LDL from the circulation. IN addition, remnant receptors are responsible for clearing chylomicrons and VLDL remnants i.e., IDL). However, the affinity of apo E for the LDL receptor is greater than that of apo B-100. As a result, the LDL particles have a much longer circulating life span than IDL particles; LDL circulates for an average of two and a half days before binding to the LDL receptors in the liver and other tissues. High serum levels of LDL, the “bad” cholesterol, are positively associated with coronary heart disease. For example, in atherosclerosis, cholesterol derived from circulating LDL accumulates in the walls of arteries. This accumulation forms bulky plaques that inhibit the flow of blood until a clot eventually forms, obstructing an artery and causing a heart attack or stroke.

Ultimately, the amount of intracellular cholesterol liberated from the LDL controls cellular cholesterol metabolism. The accumulation of cellular cholesterol derived from VLDL and LDL controls three processes. First, it reduces the cell's ability to make its own cholesterol by turning off the synthesis of HMGCoA reductase, a key enzyme in the cholesterol biosynthetic pathway. Second, the incoming LDL-derived cholesterol promotes storage of cholesterol by the action of ACAT, the cellular enzyme that converts cholesterol into cholesteryl esters that are deposited in storage droplets. Third, the accumulation of cholesterol within the cell drives a feedback mechanism that inhibits cellular synthesis of new LDL receptors. Cells, therefore, adjust their complement of LDL receptors so that enough cholesterol is brought in to meet their metabolic needs, without overloading (for a review, see Brown & Goldstein, In, The Pharmacological Basis Of Therapeutics, 8th Ed., Goodman & Gilman, Pergaman Press, NY, 1990, Ch. 36, pp. 874-896).

High levels of apo B-containing lipoproteins can be trapped in the subendothelial space of an artery and undergo oxidation. The oxidized lipoprotein is recognized by scavenger receptors on macrophages. Binding of oxidized lipoprotein to the scavenger receptors can enrich the macrophages with cholesterol and cholesteryl esters independently of the LDL receptor. Macrophages can also produce cholesteryl esters by the action of ACAT. LDL can also be complexed to a high molecular weight glycoprotein called apolipoprotein(a), also known as apo(a), through a disulfide bridge. The LDL-apo(a) complex is known as Lipoprotein(a) or Lp(a). Elevated levels of Lp(a) are detrimental, having been associated with atherosclerosis, coronary heart disease, myocardial infarcation, stroke, cerebral infarction, and restenosis following angioplasty.

### 2.3. Reverse Cholesterol Transport

Peripheral (non-hepatic) cells predominantly obtain their cholesterol from a combination of local synthesis and uptake of preformed sterol from VLDL and LDL. Cells expressing scavenger receptors, such as macrophages and smooth muscle cells, can also obtain cholesterol from oxidized apo B-containing lipoproteins. In contrast, reverse cholesterol transport (RCT) is the pathway by which peripheral cell cholesterol can be returned to the liver for recycling to extrahepatic tissues, hepatic storage, or excretion into the intestine in bile. The RCT pathway represents the only means of eliminating cholesterol from most extrahepatic tissues and is crucial to maintenance of the structure and function of most cells in the body.

The enzyme in blood involved in the RCT pathway, lecithin:cholesterol acyltransferase (LCAT), converts cell-derived cholesterol to cholesteryl esters, which are sequestered in HDL destined for removal. LCAT is produced mainly in the liver and circulates in plasma associated with the HDL fraction. Cholesterol ester transfer protein (CETP) and another lipid transfer protein, phospholipid transfer protein (PLTP), contribute to further remodeling the circulating HDL population (see for example Bruce et al., 1998, Annu. Rev. Nutr. 18:297-330). PLTP supplies lecithin to HDL, and CETP can move cholesteryl ester made by LCAT to other lipoproteins, particularly apoB-containing lipoproteins, such as VLDL. HDL triglyceride can be catabolized by the extracellular hepatic triglyceride lipase, and lipoprotein cholesterol is removed by the liver via several mechanisms.

Each HDL particle contains at least one molecule, and usually two to four molecules, of apolipoprotein (apo A-I). Apo A-I is synthesized by the liver and small intestine as preproapolipoprotein which is secreted as a proprotein that is rapidly cleaved to generate a mature polypeptide having 243 amino acid residues. Apo A-I consists mainly of a 22 amino acid repeating segment, spaced with helix-breaking proline residues. Apo A-I forms three types of stable structures with lipids: small, lipid-poor complexes referred to as pre-beta-1 HDL; flattened discoidal particles, referred to as pre-beta-2 HDL, which contain only polar lipids (e.g., phospholipid and cholesterol); and spherical particles containing both polar and nonpolar lipids, referred to as spherical or mature HDL (HDL₃ and HDL₂). Most HDL in the circulating population contains both apo A-I and apo A-II, a second major HDL protein. This apo A-1- and apo A-II-containing fraction is referred to herein as the AI/AII-HDL fraction of HDL. But the fraction of HDL containing only apo A-I, referred to herein as the AI-HDL fraction, appears to be more effective in RCT. Certain epidemiologic studies support the hypothesis that the AI-HDL fraction is antiartherogenic (Parra et al., 1992, Arterioscler. Thromb. 12:701-707; Decossin et al., 1997, Eur. J Clin. Invest. 27:299-307).

Although the mechanism for cholesterol transfer from the cell surface is unknown, it is believed that the lipid-poor complex, pre-beta-1 HDL, is the preferred acceptor for cholesterol transferred from peripheral tissue involved in RCT. Cholesterol newly transferred to pre-beta-1 HDL from the cell surface rapidly appears in the discoidal pre-beta-2 HDL. PLTP may increase the rate of disc formation (Lagrost et al., 1996, J. Biol. Chem. 271:19058-19065), but data indicating a role for PLTP in RCT is lacking. LCAT reacts preferentially with discoidal and spherical HDL, transferring the 2-acyl group of lecithin or phosphatidylethanolamine to the free hydroxyl residue of fatty alcohols, particularly cholesterol, to generate cholesteryl esters (retained in the HDL) and lysolecithin. The LCAT reaction requires an apoliprotein such apo A-I or apo A-IV as an activator. ApoA-I is one of the natural cofactors for LCAT. The conversion of cholesterol to its HDL-sequestered ester prevents re-entry of cholesterol into the cell, resulting in the ultimate removal of cellular cholesterol. Cholesteryl esters in the mature HDL particles of the AI-HDL fraction are removed by the liver and processed into bile more effectively than those derived from the AI/AII-HDL fraction. This may be due, in part, to the more effective binding of AI-HDL to the hepatocyte membrane. Several HDL receptor receptors have been identified, the most well characterized of which is the scavenger receptor class B, type I (SR-BI) (Acton et al., 1996, Science 271:518-520). The SR-BI is expressed most abundantly in steroidogenic tissues (e.g., the adrenals), and in the liver (Landshulz et al., 1996, J. Clin. Invest. 98:984-995; Rigotti et al., 1996, J. Biol. Chem. 271:33545-33549). Other proposed HDL receptors include HB1 and HB2 (Hidaka and Fidge, 1992, Biochem J. 15:161-7; Kurata et al., 1998, J. Atherosclerosis and Thrombosis 4:112-7).

While there is a consensus that CETP is involved in the metabolism of VLDL- and LDL-derived lipids, its role in RCT remains controversial. However, changes in CETP activity or its acceptors, VLDL and LDL, play a role in “remodeling” the HDL population. For example, in the absence of CETP, the HDL becomes enlarged particles that are poorly removed from the circulation (for reviews on RCT and HDLs, see Fielding & Fielding, 1995, J. Lipid Res. 36:211-228; Barrans et al., 1996, Biochem. Biophys. Acta. 1300:73-85; Hirano et al., 1997, Arterioscler. Thromb. Vasc. Biol. 17:1053-1059).

### 2.4. Reverse Transport of Other Lipids

HDL is not only involved in the reverse transport of cholesterol, but also plays a role in the reverse transport of other lipids, i.e., the transport of lipids from cells, organs, and tissues to the liver for catabolism and excretion. Such lipids include sphingomyelin, oxidized lipids, and lysophophatidylcholine. For example, Robins and Fasulo (1997, J Clin. Invest. 99:380-384) have shown that HDL stimulates the transport of plant sterol by the liver into bile secretions.

### 2.5. Peroxisome Proliferator Activated Receptor Pathway

Peroxisomes are single-membrane organelles involved in β-oxidation of a number of substrates in eukaryotic cells, such as long chain fatty acids, saturated and unsaturated very long chain fatty acids, and long chain dicarboxylic acids. A structurally diverse class of compounds called peroxisome proliferators has been characterized as anti-cholesterolemic therapeutics. When administered to test rodents, peroxisome proliferators elicit dramatic increases in the size and number of hepatic and renal peroxisomes, as well as concomitant increases in the capacity of peroxisomes to metabolize fatty acids via increased expression of the enzymes required for the β-oxidation cycle (Lazarow and Fujiki, 1985, Ann. Rev. Cell Biol. 1:489-530; Vamecq and Draye, 1989, Essays Biochem. 24:1115-225; and Nelali et al., 1988, Cancer Res. 48:5316-5324). Chemicals included in this group are the fibrate class of hypolipidermic drugs, herbicides, and phthalate plasticizers (Reddy and Lalwani, 1983, Crit. Rev. Toxicol. 12:1-58). Peroxisome proliferation can also be elicited by dietary or physiological factors, such as a high-fat diet and cold acclimatization.

Insight into the mechanism whereby peroxisome proliferators exert their pleiotropic effects was provided by the identification of a member of the nuclear hormone receptor superfamily activated by these chemicals (Isseman and Green, 1990, Nature, 347:645-650). This receptor, termed peroxisome proliferator activated receptor α (PPARα), was subsequently shown to be activated by a variety of medium and long-chain fatty acids. PPAR_{α} activates transcription by binding to DNA sequence elements, termed peroxisome proliferator response elements (PPRE), in the form of a heterodimer with the retinoid X receptor (RXR). RXR is activated by 9-cis retinoic acid (see Kliewer et al., 1992, Nature 358:771-774; Gearing et al., 1993, Proc. Natl. Acad. Sci. USA 90:1440-1444, Keller et al., 1993, Proc. Natl. Acad. Sci. USA 90:2160-2164; Heyman et al., 1992, Cell 68:397-406, and Levin et al, 1992, Nature 355:359-361). Since the discovery of PPAR_{α}, additional isoforms of PPAR have been identified, e.g., PPAR_{β}, PPAR_{γ} and PPAR_{δ}, which are have similar functions and are similarly regulated.

PPREs have been identified in the enhancers of a number of genes encoding proteins that regulate lipid metabolism. These proteins include the three enzymes required for peroxisomal β-oxidation of fatty acids; apolipoprotein A-I; medium-chain acyl-CoA dehydrogenase, a key enzyme in mitochondrial β-oxidation; and aP2, a lipid binding protein expressed exclusively in adipocytes (reviewed in Keller and Whali, 1993, TEM, 4:291-296; see also Staels and Auwerx, 1998, Atherosclerosis 137 Suppl:S19-23). The nature of the PPAR target genes coupled with the activation of PPARs by fatty acids and hypolipidemic drugs suggests a physiological role for the PPARs in lipid homeostasis.

Pioglitazone, an antidiabetic compound of the thiazolidinedione class, was reported to stimulate expression of a chimeric gene containing the enhancer/promoter of the lipid-binding protein aP2 upstream of the chloroamphenicol acetyl transferase reporter gene (Harris and Kletzien, 1994, Mol. Pharmacol. 45:439-445). Deletion analysis led to the identification of an approximately 30 bp region responsible for pioglitazone responsiveness. In an independent study, this 30 bp fragment was shown to contain a PPRE (Tontonoz et al., 1994, Nucleic Acids Res. 22:5628-5634). Taken together, these studies suggested the possibility that the thiazolidinediones modulate gene expression at the transcriptional level through interactions with a PPAR and reinforce the concept of the interrelatedness of glucose and lipid metabolism.

### 2.6. Current Cholesterol Management Therapies

In the past two decades or so, the segregation of cholesterolemic compounds into HDL and LDL regulators and recognition of the desirability of decreasing blood levels of the latter has led to the development of a number of drugs. However, many of these drugs have undesirable side effects and/or are contraindicated in certain patients, particularly when administered in combination with other drugs.

Bile-acid-binding resins are a class of drugs that interrupt the recycling of bile acids from the intestine to the liver. Examples of bile-acid-binding resins are cholestyramine (QUESTRAN LIGHT, Bristol-Myers Squibb), and colestipol hydrochloride (COLESTID, Pharmacia & Upjohn Company). When taken orally, these positively charged resins bind to negatively charged bile acids in the intestine. Because the resins cannot be absorbed from the intestine, they are excreted, carrying the bile acids with them. The use of such resins, however, at best only lowers serum cholesterol levels by about 20%. Moreover, their use is associated with gastrointestinal side-effects, including constipation and certain vitamin deficiencies. Moreover, since the resins bind to drugs, other oral medications must be taken at least one hour before or four to six hours subsequent to ingestion of the resin, complicating heart patients' drug regimens.

The statins are inhibitors of cholesterol synthesis. Sometimes, the statins are used in combination therapy with bile-acid-binding resins. Lovastatin (MEVACOR, Merck & Co., Inc.), a natural product derived from a strain of Aspergillus; pravastatin (PRAVACHOL, Bristol-Myers Squibb Co.); and atorvastatin (LIPITOR, Warner Lambert) block cholesterol synthesis by inhibiting HMGCoA, the key enzyme involved in the cholesterol biosynthetic pathway. Lovastatin significantly reduces serum cholesterol and LDL-serum levels. It also slows progression of coronary atherosclerosis. However, serum HDL levels are only slightly increased following lovastatin administration. The mechanism of the LDL-lowering effect may involve both reduction of VLDL concentration and induction of cellular expression of LDL-receptor, leading to reduced production and/or increased catabolism of LDL. Side effects, including liver and kidney dysfunction are associated with the use of these drugs.

Niacin, also known as nicotinic acid, is a water-soluble vitamin B-complex used as a dietary supplement and antihyperlipidemic agent. Niacin diminishes production of VLDL and is effective at lowering LDL. It is used in combination with bile-acid-binding resins. Niacin can increase HDL when administered at therapeutically effective doses; however, its usefulness is limited by serious side effects.

Fibrates are a class of lipid-lowering drugs used to treat various forms of hyperlipidemia, elevated serum triglycerides, which may also be associated with hypercholesterolemia. Fibrates appear to reduce the VLDL fraction and modestly increase HDL; however, the effects of these drugs on serum cholesterol is variable. In the United States, fibrates have been approved for use as antilipidemic drugs, but have not received approval as hypercholesterolemia agents. For example, clofibrate (ATROMID-S, Wyeth-Ayerst Laboratories) is an antilipidemic agent that acts to lower serum triglycerides by reducing the VLDL fraction. Although ATROMID-S may reduce serum cholesterol levels in certain patient subpopulations, the biochemical response to the drug is variable, and is not always possible to predict which patients will obtain favorable results. ATROMID-S has not been shown to be effective for prevention of coronary heart disease. The chemically and pharmacologically related drug, gemfibrozil (LOPID, Parke-Davis), is a lipid regulating agent which moderately decreases serum triglycerides and VLDL cholesterol. LOPID also increases HDL cholesterol, particularly the HDL₂ and HDL₃ sub fractions, as well as both the AI/AII-HDL fraction. However, the lipid response to LOPID is heterogeneous, especially among different patient populations. Moreover, while prevention of coronary heart disease was observed in male patients between the ages of 40 and 55 without history or symptoms of existing coronary heart disease, it is not clear to what extent these findings can be extrapolated to other patient populations (e.g., women, older and younger males). Indeed, no efficacy was observed in patients with established coronary heart disease. Serious side-effects are associated with the use of fibrates, including toxicity; malignancy, particularly malignancy of gastrointestinal cancer; gallbladder disease; and an increased incidence in non-coronary mortality. These drugs are not indicated for the treatment of patients with high LDL or low HDL as their only lipid abnormality.

Oral estrogen replacement therapy may be considered for moderate hypercholesterolemia in post-menopausal women. However, increases in HDL may be accompanied with an increase in triglycerides. Estrogen treatment is, of course, limited to a specific patient population, postmenopausal women, and is associated with serious side effects, including induction of malignant neoplasms; gall bladder disease; thromboembolic disease; hepatic adenoma; elevated blood pressure; glucose intolerance; and hypercalcemia.

Long chain carboxylic acids, particularly long chain αω-dicarboxylic acids with distinctive substitution patterns, and their simple derivatives and salts, have been disclosed for treating atherosclerosis, obesity, and diabetes (See, e.g., Bisgaier et al., 1998, J. Lipid Res. 39:17-30, and references cited therein; International Patent Publication WO 98/30530; U.S. Pat. No. 4,689,344; International Patent Publication WO 99/00116; and U.S. Pat. No. 5,756,344). However, some of these compounds, for example the α,ω′-dicarboxylic acids substituted at their α,α′-carbons (U.S. Pat. No. 3,773,946), while having serum triglyceride and serum cholesterol-lowering activities, have no value for treatment of obesity and hypercholesterolemia (U.S. Pat. No. 4,689,344).

U.S. Pat. No. 4,689,344 discloses β,β,β′,β′-tetrasubstituted-α,ω-alkanedioic acids that are optionally substituted at their α,α,β′,β′ positions, and alleges that they are useful for treating obesity, hyperlipidemia, and diabetes. According to this reference, both triglycerides and cholesterol are lowered significantly by compounds such as 3,3,14,14-tetramethylhexadecane-1,16-dioic acid. U.S. Pat. No. 4,689,344 further discloses that the β,β,β′,β′-tetramethyl-alkanediols of U.S. Pat. No. 3,930,024 also are not useful for treating hypercholesterolemia or obesity.

Other compounds are disclosed in U.S. Pat. No. 4,711,896. In U.S. Pat. No. 5,756,544, αω-dicarboxylic acid-terminated dialkane ethers are disclosed to have activity in lowering certain plasma lipids, including Lp(a), triglycerides, VLDL-cholesterol, and LDL-cholesterol, in animals, and elevating others, such as HDL-cholesterol. The compounds are also stated to increase insulin sensitivity. In U.S. Pat. No. 4,613,593, phosphates of dolichol, a polyprenol isolated from swine liver, are stated to be useful in regenerating liver tissue, and in treating hyperuricuria, hyperlipemia, diabetes, and hepatic diseases in general.

U.S. Pat. No. 4,287,200 discloses azolidinedione derivatives with anti-diabetic, hypolipidemic, and anti-hypertensive properties. However, these administration of these compounds to patients can produce side effects such as bone marrow depression, and both liver and cardiac cytotoxicity. Further, the compounds disclosed by U.S. Pat. No. 4,287,200 stimulate weight gain in obese patients.

It is clear that none of the commercially available cholesterol management drugs has a general utility in regulating lipid, lipoprotein, insulin and glucose levels in the blood. Thus, compounds that have one or more of these utilities are clearly needed. Further, there is a clear need to develop safer drugs that are efficacious at lowering serum cholesterol, increasing HDL serum levels, preventing coronary heart disease, and/or treating existing disease such as atherosclerosis, obesity, diabetes, and other diseases that are affected by lipid metabolism and/or lipid levels. There is also is a clear need to develop drugs that may be used with other lipid-altering treatment regimens in a synergistic manner. There is still a further need to provide useful therapeutic agents whose solubility and Hydrophile/Lipophile Balance (HLB) can be readily varied.

Citation or identification of any reference in Section 2 of this application is not an admission that such reference is available as prior art to the present invention.

### 3. SUMMARY OF THE INVENTION

In one embodiment, the invention relates to compounds of formula I:

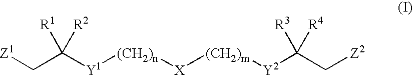

and pharmaceutically acceptable salts, solvates, hydrates, clathrates, or prodrugs thereof, wherein:

Z¹ and Z² are independently —OH, —OPO₃H, —OP₂O₆H₂, —OPO₃-(nucleotide), —OP₂O₆(H)-(nucleotide);

R¹ and R³ are independently hydrogen, methyl, or phenyl;

R² and R⁴ are independently methyl or phenyl;

m and n are independently 0, 1, 2, 3, 4, 5, or 6;

Y¹ and Y² are independently —CH₂,

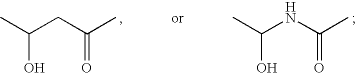

and

X is O, S, Se, C(O), C(H)F, CF₂, S(O), NH, O—P(O)(OH)—O, NH—C(O)—NH or NH—C(S)—NH.

Another embodiment encompasses compounds and pharmaceutically acceptable salts, solvates, hydrates, clathrates, or prodrugs of formula I wherein Z¹ and Z² are independently OPO₃-(nucleotide) or —OP₂O₆(H)-(nucleotide).

Another embodiment encompasses compounds and pharmaceutically acceptable salts, solvates, hydrates, clathrates, or prodrugs of formula I but with the proviso that when:

X is O;

n and m are 3;

Y¹ and Y² are —CH₂—; and

R¹-R⁴ are independently methyl or phenyl,

then at least one of Z¹ and Z² is —OPO₃-(nucleotide) or —OP₂O₆(H)-(nucleotide).

Another embodiment encompasses compounds and pharmaceutically acceptable salts, solvates, hydrates, clathrates, or prodrugs of formula I but with the proviso that when:

X is O;

n and m are 3;

Y¹ and Y² are —CH₂—; and

R¹-R⁴ are independently methyl or phenyl,

then Z¹ and Z² are indepently —OPO₃—-(nucleotide) or —OP₂O₆(H)-(nucleotide).

Another embodiment encompasses compounds and pharmaceutically acceptable salts, solvates, hydrates, clathrates, or prodrugs of formula I but with the proviso that when:

X is C(O), S, or S(O);

n and m are 1-4;

Y^{1 and Y}² are —CH₂—; and

R¹-R⁴ are independently methyl or phenyl,

then at least one of Z¹ and Z² is —OPO₃—-(nucleotide) or —OP₂O₆(H)-(nucleotide).

Another embodiment encompasses compounds and pharmaceutically acceptable salts, solvates, hydrates, clathrates, or prodrugs of formula I but with the proviso that when:

X is C(O), S, or S(O);

n and m are 1-4;

Y¹ and Y² are —CH₂—; and

R¹-R⁴ are independently methyl or phenyl,

then Z¹ and Z² is —OPO₃-(nucleotide) or —OP₂O₆(H)-(nucleotide).

In another embodiment, the invention relates to a compound of formula I:

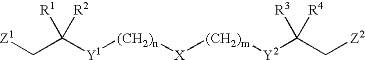

or pharmaceutically acceptable salts, solvates, hydrates, clathrates, or prodrugs thereof, wherein:

Z¹ and Z² are independently —OH, —OPO₃H, —OP₂O₆H₂, —OPO₃—-(nucleotide), —OP₂O₆(H)-(nucleotide);

R¹ and R² are taken together to form a cycloalkyl ring of 3 to 6 carbons;

R³ is hydrogen, methyl, or phenyl;

R⁴ is methyl or phenyl;

m and n are independently 0, 1, 2, 3, 4, 5, or 6;

Y¹ and Y² are independently —CH₂,

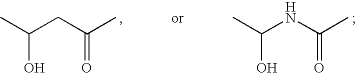

and

X is O, S, Se, C(O), C(H)F, CF₂, S(O), NH, O—P(O)(OH)—O, NH—C(O)—NH or NH—C(S)—NH.

Another embodiment encompasses compounds and pharmaceutically acceptable salts, solvates, hydrates, clathrates, or prodrugs of formula I wherein Z¹ and Z² are independently OPO₃—-(nucleotide) or —OP₂O₆(H)-(nucleotide).

In one embodiment, the compounds and pharmaceutically acceptable salts, solvates, hydrates, clathrates, or prodrugs of the compounds of formula I, but with the proviso that when:

X is O;

n and m are 3;

Y¹ and Y² are —CH₂—;

R¹ and R² are taken together to form a cycloalkyl ring of 3 to 6 carbons; and R³ and R⁴ are independently methyl or phenyl,

then at least one of Z¹ and Z² is —OPO₃—-(nucleotide) or —OP₂O₆(H)-(nucleotide).

Another embodiment encompasses compounds and pharmaceutically acceptable salts, solvates, hydrates, clathrates, or prodrugs of formula I but with the proviso that when:

X is O;

n and m are 3;

Y¹ and Y² are —CH₂—;

R¹ and R² are taken together to form a cycloalkyl ring of 3 to 6 carbons; and R³ and R⁴ are independently methyl or phenyl,

then Z¹ and Z² are independently —OPO₃-(nucleotide) or —OP₂O₆(H)-(nucleotide).

Another embodiment encompasses compounds and pharmaceutically acceptable salts, solvates, hydrates, clathrates, or prodrugs of formula I but with the proviso that when:

X is C(O), S, or S(O);

n and m are 1-4;

Y¹ and Y² are —CH₂—; and

R¹-R⁴ are independently methyl or phenyl,

then at least one of Z¹ and Z² is —OPO₃-(nucleotide) or —OP₂O₆(H)-(nucleotide).

Another embodiment encompasses compounds and pharmaceutically acceptable salts, solvates, hydrates, clathrates, or prodrugs of formula I but with the proviso that when:

X is C(O), S, or S(O);

n and m are 1-4;

Y¹ and Y² are —CH₂—; and

R¹-R⁴ are independently methyl or phenyl,

then Z¹ and Z² is —OPO₃-(nucleotide) or —OP₂O₆(H)-(nucleotide).

In another embodiment, the invention relates to compounds of formula I:

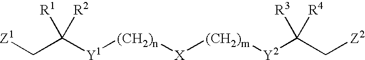

or pharmaceutically acceptable salts, solvates, hydrates, clathrates, or prodrugs thereof, wherein:

Z¹ and Z² are independently —OH, —OPO₃H, —OP₂O₆H₂, —OPO₃-(nucleotide), —OP₂O₆(H)-(nucleotide);

R¹ and R² are taken together to form a cycloalkyl ring of 3 to 6 carbons;

R³ and R⁴ are taken together to form a cycloalkyl ring of 3 to 6 carbons;

m and n are independently 0, 1, 2, 3, 4, 5, or 6;

Y¹ and Y² are independently —CH₂,

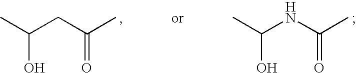

and

X is O, S, Se, C(O), C(H)F, CF₂, S(O), NH, O—P(O)(OH)—O, NH—C(O)—NH or NH—C(S)—NH.

Another embodiment encompasses compounds and pharmaceutically acceptable salts, solvates, hydrates, clathrates, or prodrugs of formula I wherein Z¹ and Z² are independently OPO₃-(nucleotide) or —OP₂O₆(H)-(nucleotide).

Another embodiment encompasses compounds and pharmaceutically acceptable salts, solvates, hydrates, clathrates, or prodrugs of formula I but with the proviso that when:

X is O;

n and m are 3;

Y¹ and Y² are —CH₂—;

R¹ and R² are taken together to form a cycloalkyl ring of 3 to 6 carbons; and R³ and R⁴ are taken together to form a cycloalkyl ring of 3 to 6 carbons,

then at least one of Z and Z² is —OPO₃-(nucleotide) or —OP₂O₆(H)-(nucleotide).

Another embodiment encompasses compounds and pharmaceutically acceptable salts, solvates, hydrates, clathrates, or prodrugs of formula I but with the proviso that when:

X is O;

n and m are 3;

Y¹ and Y² are —CH₂—;

R¹ and R² are taken together to form a cycloalkyl ring of 3 to 6 carbons; and R³ and R⁴ are taken together to form a cycloalkyl ring of 3 to 6 carbons,

then Z¹ and Z² are independently —OPO₃-(nucleotide) or —OP₂O₆(H)-(nucleotide).

Another embodiment encompasses compounds and pharmaceutically acceptable salts, solvates, hydrates, clathrates, or prodrugs of formula I but with the proviso that when:

X is C(O), S, or S(O);

n and m are 1-4;

Y¹ and Y² are —CH₂—; and

R¹-R⁴ are independently methyl or phenyl,

then at least one of Z¹ and Z² is —OPO₃-(nucleotide) or —OP₂O₆(H)-(nucleotide).

Another embodiment encompasses compounds and pharmaceutically acceptable salts, solvates, hydrates, clathrates, or prodrugs of formula I but with the proviso that when:

X is C(O), S, or S(O);

n and m are 1-4;

Y¹ and Y² are —CH₂—; and

R¹-R⁴ are independently methyl or phenyl,

then Z¹ and Z² is —OPO₃-(nucleotide) or —OP₂O₆(H)-(nucleotide).

In another embodiment, the compounds of the invention can be co-administered with a second or third active agent as described in U.S. Provisional Application No. 60/393,184, the entire disclosure of which is incorparated herein by reference.

The compounds of formula I and pharmaceutically acceptable salts, solvates, hydrates, clathrates, or prodrugs thereof are Acyl coenzyme-A mimics. Particular compounds of formula I are useful for treating or preventing cardiovascular diseases, dyslipidemias, dyslipoproteinemias, disorders of glucose metabolism, Alzheimer's Disease, Syndrome X, PPAR-associated disorders, septicemia, thrombotic disorders, obesity, pancreatitis, hypertension, renal diseases, cancer, inflammation, bacterial infection and impotence.

Particular compounds of formula I and pharmaceutically acceptable salts, solvates, hydrates, clathrates, or prodrugs thereof are useful for increasing a patient's HDL cholesterol level, lowering a patient's LDL cholesterol level, lowering a patient's VLDL cholesterol level, lowering a patient's triglyceride level, lowering a patient's insulin level, lowering a patient's glucose level, increasing a patient's ketone body level, inhibiting fatty acid synthesis in a patient, and inhibiting cholesterol synthesis in a patient.

A further embodiment of the invention provides pharmaceutical compositions comprising a compound of formula I or a pharmaceutically acceptable salt, solvate, hydrate, clathrate, or prodrug thereof, and a pharmaceutically acceptable carrier.

Compositions of the invention are useful for treating or preventing cardiovascular diseases, dyslipidemias, dyslipoproteinemias, disorders of glucose metabolism, Alzheimer's Disease, Syndrome X, PPAR-associated disorders, septicemia, thrombotic disorders, obesity, pancreatitis, hypertension, renal diseases, cancer, inflammation, bacterial infection and impotence.

Compositions of the invention are useful for increasing a patient's HDL cholesterol level, lowering a patient's LDL cholesterol level, lowering a patient's VLDL cholesterol level, lowering a patient's triglyceride level, lowering a patient's insulin level, lowering a patient's glucose level, increasing a patient's ketone body level, inhibiting fatty acid synthesis in a patient, and inhibiting cholesterol synthesis in a patient.

A further embodiment of the invention provides methods for treating or preventing a condition comprising administering to a patient in need thereof a therapeutically or prophylactically effective amount of a compound of formula or a pharmaceutically acceptable salt thereof, the condition being cardiovascular diseases, dyslipidemias, dyslipoproteinemias, disorders of glucose metabolism, Alzheimer's Disease, Syndrome X, PPAR-associated disorders, septicemia, thrombotic disorders, obesity, pancreatitis, hypertension, renal diseases, cancer, inflammation, bacterial infection or impotence.

Another embodiment of the invention provides methods for increasing a patient's HDL cholesterol level, lowering a patient's LDL cholesterol level, lowering a patient's VLDL cholesterol level, lowering a patient's triglyceride level, lowering a patient's insulin level, lowering a patient's glucose level, increasing a patient's ketone body level, inhibiting fatty acid synthesis in a patient, or inhibiting cholesterol synthesis in a patient comprising administering to a patient in need thereof a therapeutically effective amount of a compound of formula I or a pharmaceutically acceptable salt thereof

Another embodiment of the invention encompasses a method of obtaining an acyl coenzyme A mimic, comprising determining whether a test compound binds to or inhibits the activity of a fatty acid ligase, wherein a test compound that binds to or inhibits the activity of a fatty acid ligase is an acyl coenzyme A mimic.

A particular method of obtaining an acyl coenzyme A mimic, comprising comparing binding of a test compound to a short chain fatty acid ligase versus binding of a test compound to a long chain fatty acid ligase, wherein a test compound that preferentially binds to the short chain fatty acid ligase is an acyl coenzyme A mimic.

Yet another embodiment of the invention encompasses a method of obtaining an acyl coenzyme A mimic, comprising:

a. contacting a short chain fatty acid ligase with a test compound;

b. contacting a long chain fatty acid ligase with the test compound; and

c. determining whether the test compound selectively binds to or inhibits the activity of the short chain fatty acid ligase.

Another embodiment encompasses a method of obtaining an acyl coenzyme A mimic comprising comparing inhibition of a short chain fatty acid ligase by a test compound versus inhibition of the activity of a long chain fatty acid ligase by the test compound, wherein a test compound that preferentially inhibits the short chain fatty acid ligase is an acyl coenzyme A mimic.

Another embodiment encompasses a method for obtaining compounds that bind to and/or inhibit an enzyme that catalyzes the formation of, or the metabolism of an acyl coenzyme A molecule.

A preferred embodiment encompasses a method for obtaining compounds that are inhibitors of short-chain acyl-coenzyme A ligases. This method comprises the steps of (1) docking a three-dimensional structure of a test compound with a three-dimensional structure of a substrate binding site of a short-chain acyl-coenzyme A ligase and determining a first binding energy value for this interaction; and (2) docking the three-dimensional structure of the test compound with a three-dimensional structure of a substrate binding site of a long-chain acyl-coenzyme A ligase and determining a second binding energy value for this interaction. This method may further comprise determining the ratio of the first binding energy value to the second binding energy value.

Another embodiment encompasses a method for obtaining acyl coenzyme A mimics that are selective inhibitors of short-chain acyl-coenzyme A ligases in which a three-dimensional structure of a test compound is docked with a three-dimensional structure of a consensus substrate binding site derived from a set of short-chain acyl-coenzyme A ligases and determining a first binding energy value for this interaction. The three-dimensional structure of the test compound is also docked with a three-dimensional structure of a consensus substrate binding site derived from a set of long-chain acyl-coenzyme A ligases and a second binding energy value is determined. This method may further comprise the step of determining the ratio of the first binding energy value to the second binding energy value.

Another embodiment encompasses a method of obtaining compounds that are acyl coenzyme A mimics that are selective inhibitors of short-chain acyl-coenzyme A metabolizing enzymes. This method comprises docking a three-dimensional structure of a test compound with a three-dimensional structure of a substrate binding site of a short-chain acyl-coenzyme A metabolizing enzyme and determining a first binding energy value for this interaction. In addition, this method comprises docking the three-dimensional structure of the test compound with a three-dimensional structure of a substrate binding site of a long-chain acyl-coenzyme A metabolizing enzyme and determining a second binding energy value for this interaction. This method further comprises determining the ratio of the first binding energy value to the second binding energy value. If this ratio is greater than one, the test compound is deemed to be a selective inhibitor of the short-chain acyl coenzyme A ligase tested. In preferred embodiments, the ratio is at least 2, at least 10, or at least 100.

Another embodiment encompasses a method of obtaining compounds that are acyl coenzyme A mimics that are selective inhibitors of short-chain acyl-coenzyme A metabolizing enzymes in which a three-dimensional structure of a test compound is docked with a three-dimensional structure of a consensus substrate binding site derived from a set of short-chain acyl-coenzyme A metabolizing enzymes and determining a first binding energy value therefor. This method further comprises the step of docking the three-dimensional structure of the test compound with a three-dimensional structure of a consensus substrate binding site derived from a set of long-chain acyl-coenzyme A metabolizing enzymes and determining a second binding energy value this interaction. The method may also comprise determining the ratio of the first binding energy value to the second binding energy value.

Also encompassed by the invention is a method of treating or preventing a condition in a patient, comprising administering to a patient in need of such treatment or prevention, a therapeutically or prophylactically effective amount of a compound or a pharmaceutically acceptable salt thereof identified according to the methods disclosed herein for obtaining acyl coenzyme A mimics that are selective inhibitors of short-chain acyl-coenzyme A ligases and for obtaining acyl coenzyme A mimics that are selective inhibitors of short-chain acyl-coenzyme A metabolizing enzymes. In certain aspects of this embodiment, the condition to be treated or prevented is cardiovascular disease, dyslipidemia, dyslipoproetinemia, glucose metabolism disorder, Alzheimer's disease, Syndrome X or Metabolic Syndrome, septicemia, thrombotic disorder, peroxisome proliferator activated receptor associated disorder, obesity, hypertension, pancreatitis, renal disease, cancer, inflammation, bacterial infection, or impotence. Preferred patients are human.

### 4. BRIEF DESCRIPTION OF THE DRAWINGS

Various aspects of the invention can be understood with reference to the figures described below (for compound A see preferred compounds, compound 1 is the reference compound bis(6-hydroxy-5,5-dimethylhexyl)ether, and compound 2 is rosiglitazone maleate salt):

FIGS. 1A-1D. FIG. 1A shows the effect on body weight of Male Sprague-Dawley rats of treatment with Compounds A, 1 or 2 for two weeks. FIG. 1B shows the percentage change in body weight of Obese female Zucker rats of treatment with Compounds A, 1 or 2 for two weeks. FIG. 1C shows the effect on liver weight of Obese female Zucker rats of treatment with Compounds A, 1 or 2 for two weeks. FIG. 1D shows the effect on the liver weight:body weight ratio of Obese female Zucker rats of treatment with Compounds A, 1 or 2 for two weeks.

FIGS. 2A-2B. FIG. 2A shows serum glucose levels of Obese female Zucker rats following two weeks of treatment with Compounds A, 1 or 2. FIG. 2B shows serum insulin levels of Obese female Zucker rats following two weeks of treatment with Compounds A, 1 or 2.

FIGS. 3A-3C. FIG. 3A shows non-esterified fatty acid levels of Obese female Zucker rats following two weeks of treatment with Compounds A, 1 or 2. FIG. 3B shows β-hydroxy butyrate levels of Obese female Zucker rats following two weeks of treatment with Compounds A, 1 or 2. FIG. 3C shows triglyceride levels of Obese female Zucker rats following two weeks of treatment with Compounds A, 1 or 2.

FIGS. 4A-4C. FIG. 4A shows the effect of treatment of Obese female Zucker rats for two weeks with Compounds A, 1 or 2 on total serum total cholesterol. FIG. 4B shows the effect of treatment of Obese female Zucker rats for two weeks with Compounds A, 1 or 2 on low and very low density lipoprotein. FIG. 4C shows the effect of treatment of Obese female Zucker rats for two weeks with Compounds A, 1 or 2 on high density lipoprotein.

FIG. 5. FIG. 5A shows the rate of total lipid synthesis in primary rat hepatocytes upon treatment with 3 μM Compound A, 10 μM Compound A, or 10 μM Compound 1. FIG. 5B shows lipid to protein synthesis ratios in primary rat hepatocytes under the same conditions.

### 5. DETAILED DESCRIPTION OF THE INVENTION

### 5.1. Definitions and Abbreviations

Apo(a): apolipoprotein(a)

Apo A-I: apolipoprotein A-I

Apo B: apolipoprotein B

Apo E: apolipoprotein E

FH: Familial hypercholesterolemia

FCH: Familial combined hyperlipidemia

GDM: Gestational diabetes mellitus

HDL: High density lipoprotein

IDL: Intermediate density lipoprotein

IDDM: Insulin dependent diabetes mellitus

LDH: Lactate dehdyrogenase

LDL: Low density lipoprotein

Lp(a): Lipoprotein (a)

MODY: Maturity onset diabetes of the young

NIDDM: Non-insulin dependent diabetes mellitus

PPAR: Peroxisome proliferator activated receptor

RXR: Retinoid X receptor

VLDL: Very low density lipoprotein

Compounds of the invention can contain one or more chiral centers and/or double bonds and, therefore, can exist as stereoisomers, such as enantiomers, diastereomers, or geometric isomers such as double-bond isomers. According to the invention, the chemical structures depicted herein, and therefore the compounds of the invention, encompass all of the corresponding compound's enantiomers and stereoisomers, that is, both the stereomerically pure form (e.g., geometrically pure, enantiomerically pure, or diastereomerically pure) and enantiomeric and stereoisomeric mixtures.

As used herein and unless otherwise indicated, the term “therapeutically effective” refers to an amount of a compound of the invention or a pharmaceutically acceptable salt, solvate, clathrate, or prodrug thereof to cause an amelioration of a disease or disorder, or at least one discernible symptom thereof. “therapeutically effective” refers to an amount of a compound of the invention or a pharmaceutically acceptable salt, solvate, clathrate, or prodrug thereof to result in an amelioration of at least one measurable physical parameter, not necessarily discernible by the patient. In yet another embodiment, the term “therapeutically effective” refes to an amount of a compound of the invention or a pharmaceutically acceptable salt, solvate, clathrate, or prodrug thereof to inhibit the progression of a disease or disorder, either physically, e.g., stabilization of a discernible symptom, physiologically, e.g., stabilization of a physical parameter, or both. In yet another embodiment, the term “therapeutically effective” refes to an amount of a compound of the invention or a pharmaceutically acceptable salt, solvate, clathrate, or prodrug thereof resulting in delaying the onset of a disease or disorder.

In certain embodiments, the compounds and compositions of the invention are administered to an animal, preferably a human, as a preventative measure against such diseases. As used herein, the term “prophylactically effective” refers to an amount of a compound of the invention or a pharmaceutically acceptable salt, solvate, clathrate, or prodrug thereof causing a reduction of the risk of acquiring a given disease or disorder. In a preferred mode of the embodiment, the compositions of the present invention are administered as a preventative measure to an animal, preferably a human, having a genetic predisposition to a cholesterol, dyslipidemia, or related disorders including, but not limited to, cardiovascular disease; artherosclerosis; stroke; peripheral vascular disease; dyslipidemia; dyslipoproteinemia; restenosis; a disorder of glucose metabolism; Alzheimer's Disease; Syndrome X; a peroxisome proliferator activated receptor-associated disorder; septicemia; a thrombotic disorder; obesity; pancreatitis; hypertension; renal disease; cancer; inflammation; inflammatory muscle diseases, such as polymylagia rheumatica, polymyositis, and fibrositis; impotence; gastrointestinal disease; irritable bowel syndrome; inflammatory bowel disease; inflammatory disorders, such as asthma, vasculitis, ulcerative colitis, Crohn's disease, Kawasaki disease, Wegener's granulomatosis, (RA), systemic lupus erythematosus (SLE), multiple sclerosis (MS), and autoimmune chronic hepatitis; impotence; arthritis, such as rheumatoid arthritis, juvenile rheumatoid arthritis, and osteoarthritis; osteoporosis, soft tissue rheumatism, such as tendonitis; bursitis; autoimmune disease, such as systemic lupus and erythematosus; scleroderma; ankylosing spondylitis; gout; pseudogout; non-insulin dependent diabetes mellitus (NIDDM); septic shock; polycystic ovarian disease; hyperlipidemias, such as familial hypercholesterolemia (FH), familial combined hyperlipidemia (FCH); lipoprotein lipase deficiencies, such as hypertriglyceridemia, hypoalphalipoproteinemia, and hypercholesterolemia; lipoprotein abnormalities associated with diabetes; lipoprotein abnormalities associated with obesity; and lipoprotein abnormalities associated with Alzheimer's Disease. Examples of such genetic predispositions include but are not limited to the ε4 allele of apolipoprotein E, which increases the likelihood of Alzheimer's Disease; a loss of function or null mutation in the lipoprotein lipase gene coding region or promoter (e.g., mutations in the coding regions resulting in the substitutions D9N and N291 S; for a review of genetic mutations in the lipoprotein lipase gene that increase the risk of cardiovascular diseases, dyslipidemias and dyslipoproteinemias, see Hayden and Ma, 1992, Mol. Cell Biochem. 113:171-176); and familial combined hyperlipidemia and familial hypercholesterolemia. In another method of the invention, the compounds of the invention or compositions of the invention are administered as a preventative measure to a patient having a non-genetic predisposition to a cholesterol, dyslipidemia, or related disorders. Examples of such non-genetic predispositions include but are not limited to cardiac bypass surgery and percutaneous transluminal coronary angioplasty, which often lead to restenosis, an accelerated form of atherosclerosis; diabetes in women, which often leads to polycystic ovarian disease; and cardiovascular disease, which often leads to impotence. Accordingly, the compositions of the invention may be used for the prevention of one disease or disorder and concurrently treating another (e.g., prevention of polycystic ovarian disease while treating diabetes; prevention of impotence while treating a cardiovascular disease). Without being limited by theory it is believed that pantethine or a derivative thereof is effective when administered to a patient for more than thirty days. Accordingly, the invention encompasses methods of treating, preventing, or managing a cholesterol, dyslipidemia, or related disorder, which comprises administering for at least thirty days to a patient in need of such treatment, prevention, or management an effective amount of pantethine, or a derivative thereof, and a second active agent or a pharmaceutically acceptable salt, solvate, clathrate, polymorph, prodrug, or pharmacologically active metabolite thereof.

A compound of the invention is considered optically active or enantiomerically pure (i.e., substantially the R-form or substantially the S-form) with respect to a chiral center when the compound is about 90% ee (enantiomeric excess) or greater, preferably, equal to or greater than 95% ee with respect to a particular chiral center. A compound of the invention is considered to be in enantiomerically-enriched form when the compound has an enantiomeric excess of greater than about 80% ee with respect to a particular chiral center. A compound of the invention is considered diastereomerically pure with respect to multiple chiral centers when the compound is about 90% de (diastereomeric excess) or greater, preferably, equal to or greater than 95% de with respect to a particular chiral center. A compound of the invention is considered to be in diastereomerically-enriched form when the compound has an diastereomeric excess of greater than about 80% de with respect to a particular chiral center. As used herein, a racemic mixture means about 50% of one enantiomer and about 50% of is corresponding enantiomer relative to all chiral centers in the molecule. Thus, the invention encompasses all enantiomerically-pure, enantiomerically-enriched, diastereomerically pure, diastereomerically enriched, and racemic mixtures of compounds of Formula I and pharmaceutically acceptable salts thereof.

Enantiomeric and diastereomeric mixtures can be resolved into their component enantiomers or stereoisomers by well known methods, such as chiral-phase gas chromatography, chiral-phase high performance liquid chromatography, crystallizing the compound as a chiral salt complex, or crystallizing the compound in a chiral solvent. Enantiomers and diastereomers can also be obtained from diastereomerically- or enantiomerically-pure intermediates, reagents, and catalysts by well known asymmetric synthetic methods.

As used herein and unless otherwise indicated, the term “stereomerically pure” means a composition that comprises one stereoisomer of a compound and is substantially free of other stereoisomers of that compound. For example, a stereomerically pure composition of a compound having one chiral center will be substantially free of the opposite enantiomer of the compound. A stereomerically pure composition of a compound having two chiral centers will be substantially free of other diastereomers of the compound. A typical stereomerically pure compound comprises greater than about 80% by weight of stercoisomer of the compound and less than about 20% by weight of other stereoisomers the compound, more preferably greater than about 90% by weight of one stereoisomer of the compound and less than about 10% by weight of the other stereoisomers of the compound, even more preferably greater than about 95% by weight of one stereoisomer of the compound and less than about 5% by weight of the other stereoisomers of the compound, and most preferably greater than about 97% by weight of one stereoisomer of the compound and less than about 3% by weight of the other stereoisomers of the compound.

As used herein and unless otherwise indicated, the term “enantiomerically pure” means a stereomerically pure composition or compound. Enantiomeric and diastereomeric mixtures can be resolved into their component enantiomers or stereoisomers by well known methods, such as chiral-phase gas chromatography, chiral-phase high performance liquid chromatography, crystallizing the compound as a chiral salt complex, or crystallizing the compound in a chiral solvent. Enantiomers and diastereomers can also be obtained from diastereomerically- or enantiomerically-pure intermediates, reagents, and catalysts by well known asymmetric synthetic methods.

As used herein and unless otherwise indicated, the term “racemic mixture” means about 50% of one enantiomer and about 50% of is corresponding enantiomer relative to all chiral centers in the molecule. Thus, the invention encompasses all enantiomerically-pure, enantiomerically-enriched, diastereomerically pure, diastereomerically enriched, and racemic mixtures of compounds of Formula I and pharmaceutically acceptable salts thereof.

The compounds of the invention are defined herein by their chemical structures and/or chemical names. Where a compound is referred to by both a chemical structure and a chemical name, and the chemical structure and chemical name conflict, the chemical structure is determinative of the compound's identity.

As used herein and unless otherwise indicated, the term “second active agent” refers to a compound or mixture of compounds that are combined and/or administered with compounds of the invention. Examples of second active agents include, but are not limited to, statins, fibrates, glitazones, biguanides, dyslipidemic controlling compounds, small peptides of the invention, and pharmaceutically acceptable salts, solvates, prodrugs thereof, and combinations thereof.

As used herein and unless otherwise indicated, the term “third active agent” refers to a compound or mixture of compounds that are combined and/or administered with compounds of the invention and a second active agent. Specific third active agents reduce a disorder such as, but not limited to, hepatotoxicity, myopathy, cataracts, or rhabdomyolysis. Examples of third active agents include, but not limited to, bile acid-binding resins; niacin; hormones and pharmaceutically acceptable salts, solvates, prodrugs thereof, and combinations thereof.

When administered to a patient, e.g., to an animal for veterinary use or for improvement of livestock, or to a human for clinical use, the compounds of the invention are administered in isolated form or as the isolated form in a pharmaceutical composition. As used herein, “isolated” means that the compounds of the invention are separated from other components of either (a) a natural source, such as a plant or cell, preferably bacterial culture, or (b) a synthetic organic chemical reaction mixture. Preferably, via conventional techniques, the compounds of the invention are purified. As used herein, “purified” means that when isolated, the isolate contains at least 95%, preferably at least 98%, of a single ether compound of the invention by weight of the isolate.

As used herein and unless otherwise indicated, the term “pharmaceutically acceptable” means approved by a regulatory agency of the Federal or a state government or listed in the U.S. Pharmacopeia or other generally recognized pharmacopeia for use in animals, and more particularly in humans. The term “vehicle” refers to a diluent, adjuvant, excipient, or carrier with which a compound of the invention is administered. Such pharmaceutical vehicles can be liquids, such as water and oils, including those of petroleum, animal, vegetable or synthetic origin, such as peanut oil, soybean oil, mineral oil, sesame oil and the like. The pharmaceutical vehicles can be saline, gum acacia, gelatin, starch paste, talc, keratin, colloidal silica, urea, and the like. In addition, auxiliary, stabilizing, thickening, lubricating and coloring agents may be used. When administered to a patient, the compounds and compositions of the invention and pharmaceutically acceptable vehicles are preferably sterile. Water is a preferred vehicle when the compound of the invention is administered intravenously. Saline solutions and aqueous dextrose and glycerol solutions can also be employed as liquid vehicles, particularly for injectable solutions. Suitable pharmaceutical vehicles also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or pH buffering agents.

As used herein and unless otherwise indicated, the term “pharmaceutically acceptable salt(s),” includes, but is not limited to, salts of acidic or basic groups that may be present in the compounds of the invention. Compounds that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions, including but not limited to sulfuric, citric, maleic, acetic, oxalic, hydrochloride, hydrobromide, hydroiodide, nitrate, sulfate, bisulfate, phosphate, acid phosphate, isonicotinate, acetate, lactate, salicylate, citrate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. Compounds of the invention that include an amino moiety also can form pharmaceutically acceptable salts with various amino acids, in addition to the acids mentioned above. Compounds of the invention that are acidic in nature are capable of forming base salts with various pharmacologically acceptable cations. Examples of such salts include alkali metal or alkaline earth metal salts and, particularly, calcium, magnesium, sodium lithium, zinc, potassium, and iron salts.

As used herein and unless otherwise indicated, the term “pharmaceutically acceptable solvate,” means a compound of the invention or a salt thereof, that further includes a stoichiometric or non-stoichiometric amount of a solvent bound by non-covalent intermolecular forces. Preferred solvents are volatile, non-toxic, and/or acceptable for administration to humans in trace amounts. The term solvate includes hydrates and means a compound of the invention or a salt thereof, that further includes a stoichiometric or non-stoichiometric amount of water bound by non-covalent intermolecular forces and includes a mono-hydrate, dihydrate, trihydrate, tetrahydrate, and the like.

As used herein and unless otherwise indicated, the term “pharmaceutically acceptable prodrug” means a derivative of a compound that can hydrolyze, oxidize, or otherwise react under biological conditions (in vitro or in vivo) to provide the compound. Examples of prodrugs include, but are not limited to, compounds that comprise biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Other examples of prodrugs include compounds that comprise NO, NO₂, ONO, and ONO₂ moieties. Prodrugs can typically be prepared using well known methods, such as those described in 1 Burger's Medicinal Chemistry and Drug Discovery, 172 178, 949 982 (Manfred E. Wolff ed., 5th ed. 1995), and Design of Prodrugs (H. Bundgaard ed., Elselvier, New York 1985).

As used herein and unless otherwise indicated, the terms “biohydrolyzable amide,” “biohydrolyzable ester,” “biohydrolyzable carbamate,” “biohydrolyzable carbonate,” “biohydrolyzable ureide,” “biohydrolyzable phosphate” mean an amide, ester, carbamate, carbonate, ureide, or phosphate, respectively, of a compound that either: 1) does not interfere with the biological activity of the compound but can confer upon that compound advantageous properties in vivo, such as uptake, duration of action, or onset of action; or 2) is biologically inactive but is converted in vivo to the biologically active compound. Examples of biohydrolyzable esters include, but are not limited to, lower alkyl esters, lower acyloxyalkyl esters (such as acetoxylmethyl, acetoxyethyl, aminocarbonyloxy-methyl, pivaloyloxymethyl, and pivaloyloxyethyl esters), lactonyl esters (such as phthalidyl and thiophthalidyl esters), lower alkoxyacyloxyalkyl esters (such as methoxycarbonyloxy-methyl, ethoxycarbonyloxyethyl and isopropoxycarbonyloxyethyl esters), alkoxyalkyl esters, choline esters, and acylamino alkyl esters (such as acetamidomethyl esters). Examples of biohydrolyzable amides include, but are not limited to, lower alkyl amides, α amino acid amides, alkoxyacyl amides, and alkylaminoalkyl-carbonyl amides. Examples of biohydrolyzable carbamates include, but are not limited to, lower alkylamines, substituted ethylenediamines, aminoacids, hydroxyalkylamines, heterocyclic and heteroaromatic amines, and polyether amines.

As used herein and unless otherwise indicated, the term “pharmaceutically acceptable hydrate” means a compound of the invention or a salt thereof, that further includes a stoichiometric or non-stoichiometric amount of water bound by non-covalent intermolecular forces.

As used herein and unless otherwise indicated, the term “pharmaceutically acceptable clathrate” means a compound of the invention or a salt thereof in the form of a crystal lattice that contains spaces (e.g., channels) that have a guest molecule (e.g., a solvent or water) trapped within.

As used herein and unless otherwise indicated, the term “altering lipid metabolism” indicates an observable (measurable) change in at least one aspect of lipid metabolism, including but not limited to total blood lipid content, blood HDL cholesterol, blood LDL cholesterol, blood VLDL cholesterol, blood triglyceride, blood Lp(a), blood apo A-I, blood apo E and blood non-esterified fatty acids.

As used herein and unless otherwise indicated, the term “altering glucose metabolism” indicates an observable (measurable) change in at least one aspect of glucose metabolism, including but not limited to total blood glucose content, blood insulin, the blood insulin to blood glucose ratio, insulin sensitivity, and oxygen consumption.

As used herein and unless otherwise indicated, the terms “alkyl group” and “(C₁-C₆)alkyl”means a saturated, monovalent unbranched or branched hydrocarbon chain. Examples of alkyl groups include, but are not limited to, (C₁-C₆)alkyl groups, such as methyl, ethyl, propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, and hexyl, and longer alkyl groups, such as heptyl, and octyl. An alkyl group can be unsubstituted or substituted with one or two suitable substituents.

As used herein and unless otherwise indicated, the term “alkenyl group” means a monovalent unbranched or branched hydrocarbon chain having one or more double bonds therein. The double bond of an alkenyl group can be unconjugated or conjugated to another unsaturated group. Suitable alkenyl groups include, but are not limited to (C₂-C₆)alkenyl groups, such as vinyl, allyl, butenyl, pentenyl, hexenyl, butadienyl, pentadienyl, hexadienyl, 2-ethylhexenyl, 2-propyl-2-butenyl, 4-(2-methyl-3-butene)-pentenyl. An alkenyl group can be unsubstituted or substituted with one or two suitable substituents.

As used herein and unless otherwise indicated, the term “alkynyl group” means monovalent unbranched or branched hydrocarbon chain having one or more triple bonds therein. The triple bond of an alkynyl group can be unconjugated or conjugated to another unsaturated group. Suitable alkynyl groups include, but are not limited to, (C₂-C₆)alkynyl groups, such as ethynyl, propynyl, butynyl, pentynyl, hexynyl, methylpropynyl, 4-methyl-1-butynyl, 4-propyl-2-pentynyl, and 4-butyl-2-hexynyl. An alkynyl group can be unsubstituted or substituted with one or two suitable substituents.

As used herein and unless otherwise indicated, the terms “aryl group” and “(C₆-C₁₄)aryl” mean a monocyclic or polycyclic-aromatic radical comprising carbon and hydrogen atoms. Examples of suitable aryl groups include, but are not limited to, phenyl, tolyl, anthacenyl, fluorenyl, indenyl, azulenyl, and naphthyl, as well as benzo-fused carbocyclic moieties such as 5,6,7,8-tetrahydronaphthyl. An aryl group can be unsubstituted or substituted with one or two suitable substituents. Preferably, the aryl group is a monocyclic ring, wherein the ring comprises 6 carbon atoms, referred to herein as “(C₆)aryl”.

As used herein and unless otherwise indicated, the term “heteroaryl group” means a monocyclic- or polycyclic aromatic ring comprising carbon atoms, hydrogen atoms, and one or more heteroatoms, preferably 1 to 3 heteroatoms, independently selected from nitrogen, oxygen, and sulfur. Illustrative examples of heteroaryl groups include, but are not limited to, pyridinyl, pyridazinyl, pyrimidinyl, pyrazyl, triazinyl, pyrrolyl, pyrazolyl, imidazolyl, (1,2,3)- and (1,2,4)-triazolyl, pyrazinyl, pyrimidinyl, tetrazolyl, furyl, thiophenyl, isoxazolyl, thiazolyl, furyl, phenyl, isoxazolyl, and oxazolyl. A heteroaryl group can be unsubstituted or substituted with one or two suitable substituents. Preferably, a heteroaryl group is a monocyclic ring, wherein the ring comprises 2 to 5 carbon atoms and 1 to 3 heteroatoms, referred to herein as “(C₂-C₅)heteroaryl”.

As used herein and unless otherwise indicated, the term “cycloalkyl group” means a monocyclic or polycyclic saturated ring comprising carbon and hydrogen atoms and having no carbon-carbon multiple bonds. Examples of cycloalkyl groups include, but are not limited to, (C₃-C₇)cycloalkyl groups, such as cyclopropyl, cyclobutyl, cyclopentyl, cyclohexyl, and cycloheptyl, and saturated cyclic and bicyclic terpenes. A cycloalkyl group can be unsubstituted or substituted by one or two suitable substituents. Preferably, the cycloalkyl group is a monocyclic ring or bicyclic ring.

As used herein and unless otherwise indicated, the term “heterocycloalkyl group” means a monocyclic or polycyclic ring comprising carbon and hydrogen atoms and at least one heteroatom, preferably, 1 to 3 heteroatoms selected from nitrogen, oxygen, and sulfur, and having no unsaturation. Examples of heterocycloalkyl groups include pyrrolidinyl, pyrrolidino, piperidinyl, piperidino, piperazinyl, piperazino, morpholinyl, morpholino, thiomorpholinyl, thiomorpholino, and pyranyl. A heterocycloalkyl group can be unsubstituted or substituted with one or two suitable substituents. Preferably, the heterocycloalkyl group is a monocyclic or bicyclic ring, more preferably, a monocyclic ring, wherein the ring comprises from 3 to 6 carbon atoms and form 1 to 3 heteroatoms, referred to herein as (C₁-C₆)heterocycloalkyl.

As used herein and unless otherwise indicated, the term “heterocyclic radical” or “heterocyclic ring” means a heterocycloalkyl group or a heteroaryl group.

As used herein and unless otherwise indicated, the term “alkoxy group”means an —O-alkyl group, wherein alkyl is as defined above. An alkoxy group can be unsubstituted or substituted with one or two suitable substituents. Preferably, the alkyl chain of an alkyloxy group is from 1 to 6 carbon atoms in length, referred to herein as “(C₁-C₆)alkoxy”.

As used herein and unless otherwise indicated, the term “aryloxy group” means an —O-aryl group, wherein aryl is as defined above. An aryloxy group can be unsubstituted or substituted with one or two suitable substituents. Preferably, the aryl ring of an aryloxy group is a monocyclic ring, wherein the ring comprises 6 carbon atoms, referred to herein as “(C₆)aryloxy”.

As used herein and unless otherwise indicated, the term “benzyl” means —CH₂-phenyl.

As used herein and unless otherwise indicated, the term “phenyl” means —C₆H₅. A phenyl group can be unsubstituted or substituted with one or two suitable substituents.

As used herein and unless otherwise indicated, the term “hydrocarbyl” group means a monovalent group selected from (C₁-C₈)alkyl, (C₂-C₈)alkenyl, and (C₂-C₈)alkynyl, optionally substituted with one or two suitable substituents. Preferably, the hydrocarbon chain of a hydrocarbyl group is from 1 to 6 carbon atoms in length, referred to herein as “(C₁₋C₆)hydrocarbyl”.

As used herein and unless otherwise indicated, the term “carbonyl” group is a divalent group of the formula —C(O)—.

As used herein and unless otherwise indicated, the term “alkoxycarbonyl” group means a monovalent group of the formula —C(O)-alkoxy. Preferably, the hydrocarbon chain of an alkoxycarbonyl group is from 1 to 8 carbon atoms in length, referred to herein as a “lower alkoxycarbonyl” group.

As used herein and unless otherwise indicated, the term “carbamoyl” group means the radical —C(O)N(R′)₂, wherein R′ is chosen from the group consisting of hydrogen, alkyl, and aryl.

As used herein and unless otherwise indicated, the term “halogen” means fluorine, chlorine, bromine, or iodine. Correspondingly, the meaning of the terms “halo” and “Hal”encompass fluoro, chloro, bromo, and iodo.

As used herein and unless otherwise indicated, the term “suitable substituent” means a group that does not nullify the synthetic or pharmaceutical utility of the compounds of the invention or the intermediates useful for preparing them. Examples of suitable substituents include, but are not limited to: (C1-C8)alkyl; (C₁-Cg)alkenyl; (C₁-C₈)alkynyl; (C₆)aryl; (C₂-C₅)heteroaryl; (C₃-C₇)cycloalkyl; (C₁-C₈)alkoxy; (C₆)aryloxy; —CN; —OH; oxo; halo, —CO₂H; —NH₂; —NH((C₁-C₈)alkyl); —N((C₁-C₈)alkyl)₂; —NH((C₆)aryl); —N((C₆)aryl)₂; —CHO; —CO((C₁-C₈)alkyl); —CO((C₆)aryl); —CO₂((C₁-C₈)alkyl); and —CO₂((C₆)aryl). One of skill in the art can readily choose a suitable substituent based on the stability and pharmacological and synthetic activity of the compound of the invention.

As used herein and unless otherwise indicated, the term “nucleotide” means a group having aribose or deoxyribose sugar joined to a purine or pyrimidine base and to one or more phosphate groups. Examples of nucleotides include, but are not limited to, adenine, guanine, cytosine, thymine, uracil and thio and thiotriphosphate analogs thereof.

As used herein and unless otherwise indicated, the term “short chain acyl coenzyme A” ligase refers to an enzyme catalyzing the condensation of a C₂-C₈ carboxylic acid and coenzyme A to form a short chain acyl-coenzyme A product. Similarly, the phrase “medium chain acyl coenzyme A” ligase refers to an enzyme catalyzing the condensation of a C₁₀-C₁₆ carboxylic acid and coenzyme A to form a short chain acyl-coenzyme A product. Accordingly, the phrase “long chain acyl coenzyme A” ligase refers to an enzyme catalyzing the condensation of a carboxylic acid comprising a carbon chain of more than 16 carbon atoms and coenzyme A to form a long chain acyl-coenzyme A product.

As used herein and unless otherwise indicated, the phrases “long chain acyl coenzyme A” metabolizing enzyme, “medium chain acyl coenzyme A” metabolizing enzyme, and “long chain acyl coenzyme A” metabolizing enzyme refer to enzymes using a short-chain, medium-chain, long-chain acyl coenzyme A molecule as a substrate, respectively.

As used herein and unless otherwise indicated, the term “docking” refers to a computer-assisted method for determining and evaluating energetically-favorable interactions between a biological macromolecule and a ligand the interacts with that biological macromolecule. As used herein, the term ligand encompasses both natural substrates as well as non-substrate inhibitors of the biochemical activity of the biological macromolecule to which it binds.

### 5.2. Compounds of Formula I

In one embodiment, the invention relates to compounds of formula I:

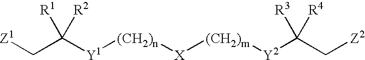

and pharmaceutically acceptable salts thereof, wherein:

Z¹ and Z² are independently —OH, —OPO₃H, —OP₂O₆H₂, —OPO₃-(nucleotide), —OP₂O₆(H)-(nucleotide);

R¹ and R³ are independently hydrogen, methyl, or phenyl;

R² and R⁴ are independently methyl or phenyl;

m and n are independently 0, 1, 2, 3, 4, 5, or 6;

Y¹ and Y² are independently —CH₂,

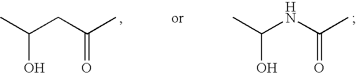

and

X is O, S, Se, C(O), C(H)F, CF₂, S(O), NH, N(OH), O—P(O)(OH)—O, NH—C(O)—NH or NH—C(S)—NH.

Another embodiment encompasses compounds and pharmaceutically acceptable salts of formula I wherein Z¹ and Z² are independently OPO₃-(nucleotide) or —OP₂O₆(H)-(nucleotide).

Another embodiment encompasses compounds and pharmaceutically acceptable salts of formula I but with the proviso that when:

X is O;

n and m are 3;

Y¹ and Y² are —CH₂—; and

R¹-R⁴ are independently methyl or phenyl,

then at least one of Z¹ and Z² is —OPO₃-(nucleotide) or —OP₂O₆(H)-(nucleotide).

Another embodiment encompasses compounds and pharmaceutically acceptable salts of formula I but with the proviso that when:

X is O;

n and m are 3;

Y¹ and Y² are —CH₂—; and

R¹-R⁴ are independently methyl or phenyl,

then Z¹ and Z² are indepently —OPO₃-(nucleotide) or —OP₂O₆(H)-(nucleotide).

Another embodiment encompasses compounds and pharmaceutically acceptable salts of formula I but with the proviso that when:

X is C(O);

n and m are 1-4;

Y¹ and Y² are —CH₂—; and

R¹-R⁴ are independently methyl or phenyl,

then at least one of Z¹ and Z² is —OPO₃-(nucleotide) or —OP₂O₆(H)-(nucleotide).

Another embodiment encompasses compounds and pharmaceutically acceptable salts of formula I but with the proviso that when:

X is C(O);

n and m are 1-4;

Y¹ and Y² are —CH₂—; and

R¹-R⁴ are independently methyl or phenyl,

then Z¹ and Z² is —OPO₃-(nucleotide) or —OP₂O₆(H)-(nucleotide).

Another embodiment encompasses compounds and pharmaceutically acceptable salts of formula I but with the proviso that when:

X is S;

n and m are 1-4;

Y¹ and Y² are —CH₂—; and

R¹-R⁴ are independently methyl or phenyl,

then at least one of Z¹ and Z² is —OPO₃-(nucleotide) or —OP₂O₆(H)-(nucleotide).

Another embodiment encompasses compounds and pharmaceutically acceptable salts of formula I but with the proviso that when:

X is S;

n and m are 1-4;

Y¹ and Y² are —CH₂—; and

R¹-R⁴ are independently methyl or phenyl,

then Z¹ and Z² is —OPO₃-(nucleotide) or —OP₂O₆(H)-(nucleotide).

Another embodiment encompasses compounds and pharmaceutically acceptable salts of formula I but with the proviso that when:

X is S(O);

n and m are 1-4;

Y¹ and Y² are —CH₂—; and

R¹-R⁴ are independently methyl or phenyl,

then at least one of Z¹ and Z² is —OPO₃-(nucleotide) or —OP₂O₆(H)-(nucleotide).

Another embodiment encompasses compounds and pharmaceutically acceptable salts of formula I but with the proviso that when:

X is S(O);

n and m are 1-4;

Y¹ and Y² are —CH₂—; and

R¹-R⁴ are independently methyl or phenyl,

then Z¹ and Z² is —OPO₃-(nucleotide) or —OP₂O₆(H)-(nucleotide).

In another preferred embodiment, Y¹ and Y² are

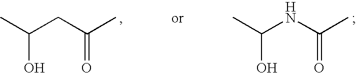

and

In another preferred embodiment, Y¹ and Y² are —CH₂ and n and m are 5 or 6.

In a still preferred embodiment, Z¹ and Z² are the same, R¹ and R³ are the same, R² and R⁴ are the same, Y¹ and Y² are the same and n and m are the same.

In another embodiment, the invention relates to a compound of formula I:

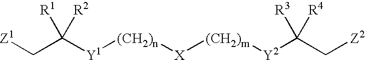

or a pharmaceutically acceptable salt thereof, wherein:

Z¹ and Z² are independently —OH, —OPO₃H, —OP₂O₆H₂, —OPO₃-(nucleotide), —OP₂O₆(H)-(nucleotide);

R¹ and R² are taken together to form a cycloalkyl ring of 3 to 6 carbons;

R³ is hydrogen, methyl, or phenyl;

R⁴ is methyl or phenyl;

m and n are independently 0, 1, 2, 3, 4, 5, or 6;

Y¹ and Y are independently —CH₂,

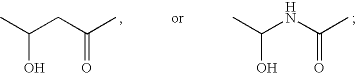

and

X is O, S, Se, C(O), C(H)F, CF₂, S(O), NH, O—P(O)(OH)—O, NH—C(O)—NH or NH—C(S)—NH.

Another embodiment encompasses compounds and pharmaceutically acceptable salts of formula I wherein Z¹ and Z² are independently OPO₃-(nucleotide) or —OP₂O₆(H)-(nucleotide).

Another embodiment encompasses compounds and pharmaceutically acceptable salts of formula I but with the proviso that when:

X is O;

n and m are 3;

Y¹ and Y² are —CH₂—; and

R¹-R⁴ are independently methyl or phenyl,

then at least one of Z¹ and Z² is —OPO₃-(nucleotide) or —OP₂O₆(H)-(nucleotide).

Another embodiment encompasses compounds and pharmaceutically acceptable salts of formula I but with the proviso that when:

X is O;

n and m are 3;

Y¹ and Y² are —CH₂—; and

R¹-R⁴ are independently methyl or phenyl,

then Z¹ and Z² are indepently —OPO₃-(nucleotide) or —OP₂O₆(H)-(nucleotide).

Another embodiment encompasses compounds and pharmaceutically acceptable salts of formula I but with the proviso that when:

X is C(O);

n and m are 1-4;

Y¹ and Y² are —CH₂—; and

R¹-R⁴ are independently methyl or phenyl,

then at least one of Z¹ and Z² is —OPO₃-(nucleotide) or —OP₂O₆(H)-(nucleotide).

Another embodiment encompasses compounds and pharmaceutically acceptable salts of formula I but with the proviso that when:

X is C(O);

n and m are 1-4;

Y¹ and Y² are —CH₂—; and

R¹-R⁴ are independently methyl or phenyl,

then Z¹ and Z² is —OPO₃-(nucleotide) or —OP₂O₆(H)-(nucleotide).

Another embodiment encompasses compounds and pharmaceutically acceptable salts of formula I but with the proviso that when:

X is S;

n and m are 1-4;

Y¹ and Y² are —CH₂—; and

R¹-R⁴ are independently methyl or phenyl,

then at least one of Z¹ and Z² is —OPO₃-(nucleotide) or —OP₂O₆(H)-(nucleotide).

Another embodiment encompasses compounds and pharmaceutically acceptable salts of formula I but with the proviso that when:

X is S;

n and m are 1-4;

Y¹ and Y² are —CH₂—; and

R¹-R⁴ are independently methyl or phenyl,

then Z¹ and Z² is —OPO₃-(nucleotide) or —OP₂O₆(H)-(nucleotide).

Another embodiment encompasses compounds and pharmaceutically acceptable salts of formula I but with the proviso that when:

X is S(O);

n and m are 1-4;

Y¹ and Y² are —CH₂—; and

R¹-R⁴ are independently methyl or phenyl,

then at least one of Z¹ and Z² is —OPO₃-(nucleotide) or —OP₂O₆(H)-(nucleotide).

Another embodiment encompasses compounds and pharmaceutically acceptable salts of formula I but with the proviso that when:

X is S(O);

n and m are 1-4;

Y¹ and Y² are —CH₂—; and

R¹-R⁴ are independently methyl or phenyl,

then Z¹ and Z² is —OPO₃-(nucleotide) or —OP₂O₆(H)-(nucleotide).

In another preferred embodiment, Y¹ and Y² are

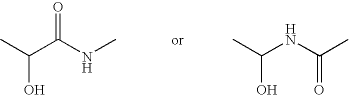

In another preferred embodiment, Y¹ and Y² are —CH₂ and n and m are 5 or 6. In another embodiment, the invention relates to compounds of formula I:

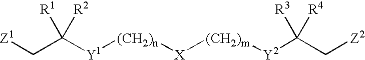

or pharmaceutically acceptable salts thereof, wherein:

Z¹ and Z² are independently —OH, —OPO₃H, —OP₂O₆H₂, —OPO₃-(nucleotide), —OP₂O₆(H)-(nucleotide);

R¹ and R² are taken together to form a cycloalkyl ring of 3 to 6 carbons;

R³ and R⁴ are taken together to form a cycloalkyl ring of 3 to 6 carbons;

m and n are independently 0, 1, 2, 3, 4, 5, or 6;

Y¹ and Y² are independently —CH₂,

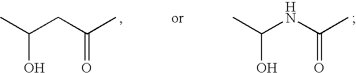

and

X is O, S, Se, C(O), C(H)F, CF₂, S(O), NH, O—P(O)(OH)—O, NH—C(O)—H, or NH—C(S)—NH.

Another embodiment encompasses compounds and pharmaceutically acceptable salts of formula I wherein Z¹ and Z² are independently OPO₃-(nucleotide) or —OP₂O₆(H)-(nucleotide).

Another embodiment encompasses compounds and pharmaceutically acceptable salts of formula I but with the proviso that when:

X is O;

n and m are 3;

Y¹ and Y² are —CH₂—; and

R¹-R⁴ are independently methyl or phenyl,

then at least one of Z¹ and Z² is —OPO₃-(nucleotide) or —OP₂O₆(H)-(nucleotide).

Another embodiment encompasses compounds and pharmaceutically acceptable salts of formula I but with the proviso that when:

X is O;

n and m are 3;

Y¹ and Y² are —CH₂—; and

R¹-R⁴ are independently methyl or phenyl,

then Z¹ and Z² are indepently —OPO₃-(nucleotide) or —OP₂O₆(H)-(nucleotide).

Another embodiment encompasses compounds and pharmaceutically acceptable salts of formula I but with the proviso that when:

X is C(O);

n and m are 1-4;

Y¹ and Y² are —CH₂—; and

R¹-R⁴ are independently methyl or phenyl,

then at least one of Z¹ and Z² is —OPO₃-(nucleotide) or —OP₂O₆(H)-(nucleotide).

Another embodiment encompasses compounds and pharmaceutically acceptable salts of formula I but with the proviso that when:

X is C(O);

n and m are 1-4;

Y¹ and Y² are —CH₂—; and

R¹-R⁴ are independently methyl or phenyl,

then Z¹ and Z² is —OPO₃-(nucleotide) or —OP₂O₆(H)-(nucleotide).

Another embodiment encompasses compounds and pharmaceutically acceptable salts of formula I but with the proviso that when:

X is S;

n and m are 1-4;

Y¹ and Y² are —CH₂—; and

R¹-R⁴ are independently methyl or phenyl,

then at least one of Z¹ and Z² is —OPO₃-(nucleotide) or —OP₂O₆(H)-(nucleotide).

Another embodiment encompasses compounds and pharmaceutically acceptable salts of formula I but with the proviso that when:

X is S;

n and m are 1-4;

Y¹ and Y² are —CH₂—; and

R¹-R⁴ are independently methyl or phenyl,

then Z¹ and Z² is —OPO₃-(nucleotide) or —OP₂O₆(H)-(nucleotide).

Another embodiment encompasses compounds and pharmaceutically acceptable salts of formula I but with the proviso that when:

X is S(O);

n and m are 1-4;

Y¹ and Y² are —CH₂—; and

R¹-R⁴ are independently methyl or phenyl,

then at least one of Z¹ and Z² is —OPO₃-(nucleotide) or —OP₂O₆(H)-(nucleotide).

Another embodiment encompasses compounds and pharmaceutically acceptable salts of formula I but with the proviso that when:

X is S(O);

n and m are 1-4;

Y¹ and Y² are —CH₂—; and

R¹-R⁴ are independently methyl or phenyl,

then Z¹ and Z² is —OPO₃-(nucleotide) or —OP₂O₆(H)-(nucleotide).

In another preferred embodiment, Y¹ and Y² are

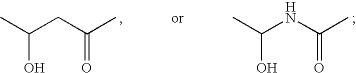

and

In another preferred embodiment, Y¹ and Y² are —CH₂ and n and m are 5 or 6.

In a still preferred embodiment, Z¹ and Z² are the same, R¹ and R² are taken together, R³ and R⁴ are taken together, Y¹ and Y² are the same and n and m are the same.

### 5.3. Illustrative Compounds of Formula I

Illustrative compounds of formula I include, but are not limited to:

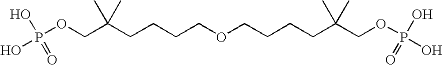

### Phosphoric Acid mono-[6-(5,5-dimethyl-6-phosphonooxy-hexyloxy)-2,2-dimethyl-hexyl] Ester

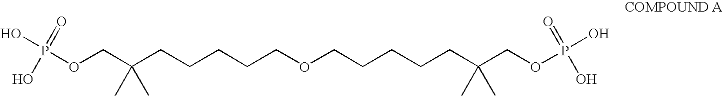

### Phosphoric Acid mono-[6-(5,5-dimethyl-6-phosphonooxy-heptyloxy)-2,2-dimethyl-hexyl] Ester

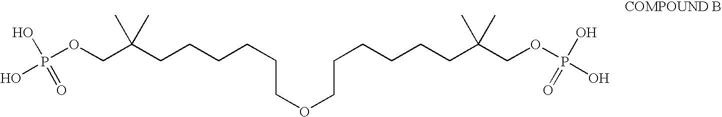

### Phosphoric Acid mono-[7,7-dimethyl-7-phosphonooxy-octyloxy)-2,2-dimethyl-octyl] Ester

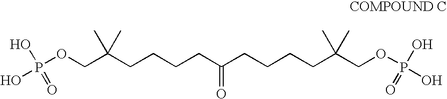

### Phosphoric Acid mono-[8-(7-oxo-13-phosphonooxy-tridecyl) Ester

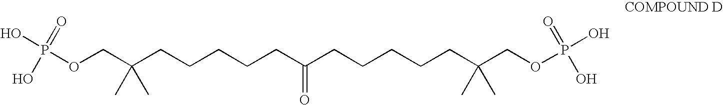

### Phosphoric Acid mono-(2,2,12,12-tetramethyl-8-oxo-13-phosphonooxy-tridecyl) Ester

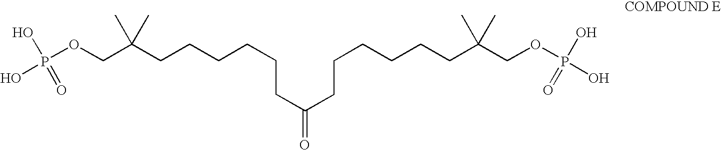

### Phosphoric Acid mono-(2,2,14,14-tetramethyl-9-oxo-15-phosphonooxy-pentadecyl) Ester

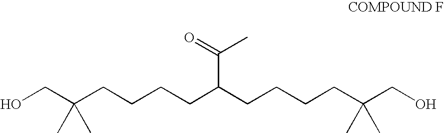

### 2,2,12,12-Tetramethyl-7-acetyltridecan-1,13-diol

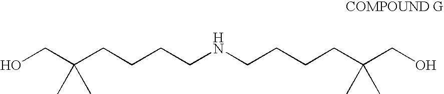

### Bis(5.5-dimethyl-6-6hydroxy-hexyl)amine hydrochloride

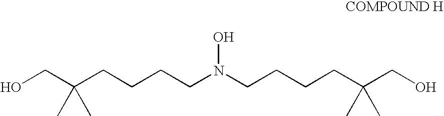

### 6-[Hydroxy-(6-hydroxy-5,5-dimethyl-hexyl)-amino]-2,2-dimethyl-hexan-1-ol

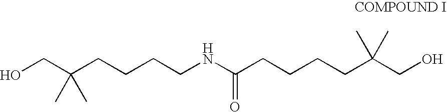

### 7-Hydroxy-6,6-dimethyl-heptanoic acid (6-hydroxy-5,5-dimethyl-hexyl)-amide

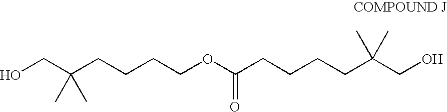

### 7-Hydroxy-6,6-dimethyl-heptanoic acid 6-hydroxy-5,5-dimethyl-hexyl Ester

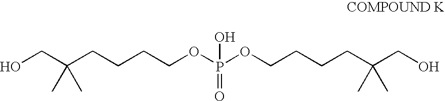

### Phosphoric Acid bis-(5,5-dimethyl-6-hydroxy-hexyl)-ester

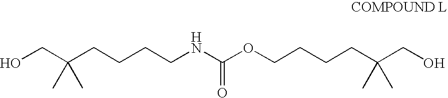

### N-(5,5-dimethyl-6-hydroxyhexyl)-O-(5,5-dimethyl-6-hydroxy)carbamate

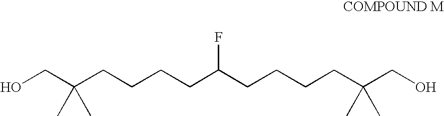

### 7-Fluoro-2,2,12,12-tetramethyl-tridecane-1,13-diol

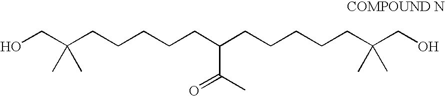

### 10-Hydroxy-3-(7-hydroxy-6,6-dimethyl-heptyl)-9,9-dimethyl-decan-2-one

### Compound O

### Error! Objects Cannot be Created from Editing Field Codes

### 7-(7-Hydroxy-6,6-dimethyl-heptylamino)-2,2-dimethyl-heptan-1-ol

### Compound P

### Error! Objects Cannot be Created from Editing Field Codes

### 7-(7-Hydroxy-6,6-dimethyl-heptylamino)-2,2-dimethyl-heptan-1-ol

### Compound Q

### Error! Objects Cannot be Created from Editing Field Codes

### 8-Hydroxy-7,7-dimethyl-octanoic Acid (7-hydroxy-6,6-dimethyl-heptyl)-amide

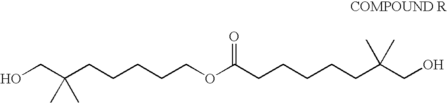

### 8-Hydroxy-7,7-dimethyl-octanoic acid 7-hydroxy-6,6-dimethyl-heptyl Ester

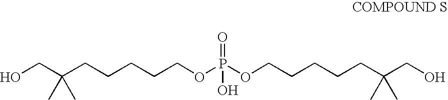

### Phosphoric Acid bis-(7-hydroxy-6,6-dimethyl-heptyl) Ester

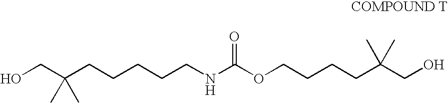

### (7-Hydroxy-6,6-dimethyl-heptyl)-carbamic acid 6-hydroxy-5,5-dimethyl-hexyl Ester

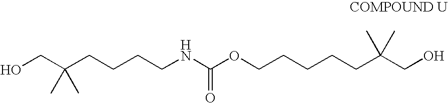

### (6-Hydroxy-5,5-dimethyl-hexyl)-carbamic Acid 7-hydroxy-6,6-dimethyl-heptyl Ester

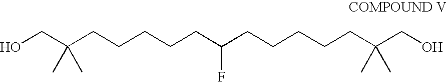

### 8-Fluoro-2,2,14,14-tetramethyl-pentadecane-1,15-diol

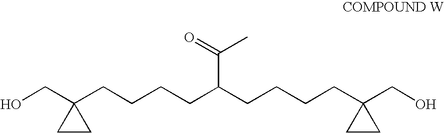

### 7-(1-Hydroxymethyl-cyclopropyl)-3-[4-(1-hydroxymethyl-cyclopropyl)-butyl]-heptan-2-one

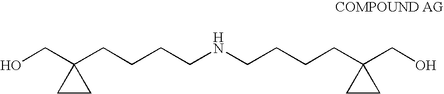

### (1-{4-[4-(1-Hydroxymethyl-cyclopropyl)-butylamino]-butyl}-cyclopropyl)-methanol

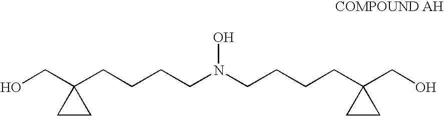

### 6[1-(4-{Hydroxy-[4-(1-hydroxymethyl-cyclopropyl)-butyl]-amino}-butyl)-cyclopropyl]-methanol

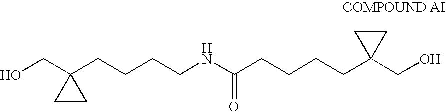

### 5-(1-Hydroxymethyl-cyclopropyl)-pentanoic Acid [4-(1-hydroxymethyl-cyclopropyl)-butyl]-amide

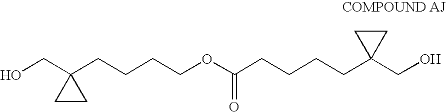

### 5-(1-Hydroxymethyl-cyclopropyl)-pentanoic Acid 4-(1-hydroxymethyl-cyclopropyl)-butyl Ester

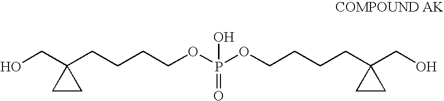

Phosphoric acid bis-[4-(1-hydroxymethyl-cyclopropyl)-butyl] Ester

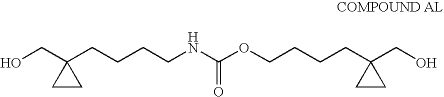

### [4-(1-Hydroxymethyl-cyclopropyl)-butyl]-carbamic acid 4-(1-hydroxymethyl-cyclopropyl)-butyl ester

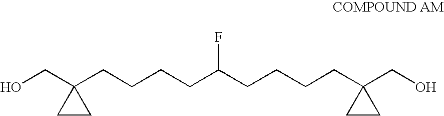

{1-[5-Fluoro-9-(1-hydroxymethyl-cyclopropyl)-nonyl]-cyclopropyl}-methanol

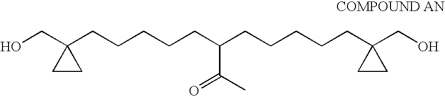

### 8-(1-Hydroxymethyl-cyclopropyl)-3-[5-(1-hydroxymethyl-cyclopropyl)-pentyl]-octan-2-one

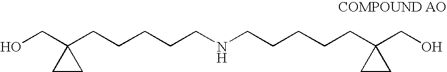

### (1-{5-[5-(1-Hydroxymethyl-cyclopropyl)-pentylamino]-pentyl}-cyclopropyl)-methanol

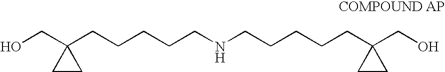

### (1-{5-[5-(1-Hydroxymethyl-cyclopropyl)-pentylamino]-pentyl}-cyclopropyl)-methanol

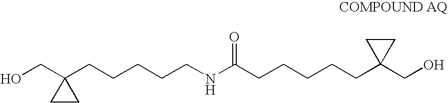

### 6-(1-Hydroxymethyl-cyclopropyl)-hexanoic acid [5-(1-hydroxymethyl-cyclopropyl)-pentyl]-amide

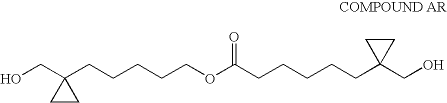

### 6-(1-Hydroxymethyl-cyclopropyl)-hexanoic Acid 5-(1-hydroxymethyl-cyclopropyl)-pentyl Ester

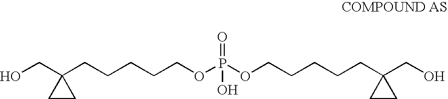

### Phosphoric Acid bis-[5-(1-hydroxymethyl-cyclopropyl)-pentyl] Ester

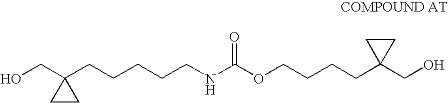

### [5-(1-Hydroxymethyl-cyclopropyl)-pentyl]-carbamic acid 4-(1-hydroxymethyl-cyclopropyl)-butyl Ester

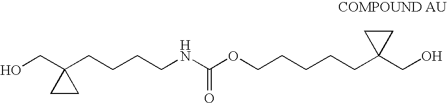

### [4-(1-Hydroxymethyl-cyclopropyl)-butyl]-carbamic Acid 5-(1-hydroxymethyl-cyclopropyl)-pentyl Ester

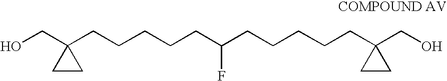

{1-[6-Fluoro-11-(1-hydroxymethyl-cyclopropyl)-undecyl]-cyclopropyl}-methanol

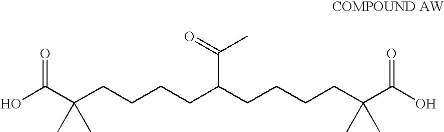

7-Acetyl-2,2,12,12-tetramethyl-tridecanedioic Acid

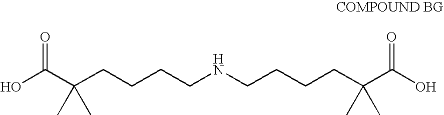

6-(5-Carboxy-5-methyl-hexylamino)-2,2-dimethyl-hexanoic Acid

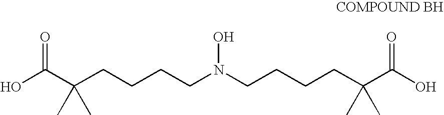

### 6-[(5-Carboxy-5-methyl-hexyl)-hydroxy-amino]-2,2-dimethyl-hexanoic Acid

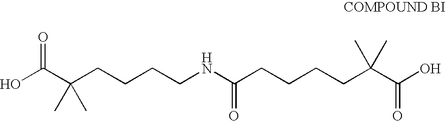

### 6-(6-Carboxy-6-methyl-heptanoylamino)-2,2-dimethyl-hexanoic Acid

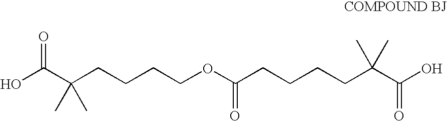

### 2,2-Dimethyl-heptanedioic Acid 7-(5-carboxy-5-methyl-hexyl) Ester

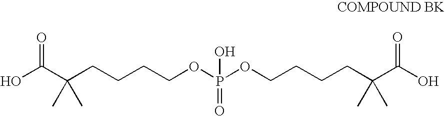

### 6-[(5-Carboxy-5-methyl-hexyloxy)-hydroxy-phosphoryloxy]-2,2-dimethyl-hexanoic Acid

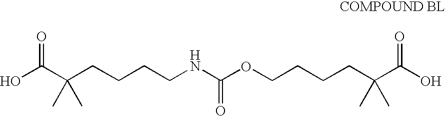

### 6-(5-Carboxy-5-methyl-hexyloxycarbonylamino)-2,2-dimethyl-hexanoic Acid

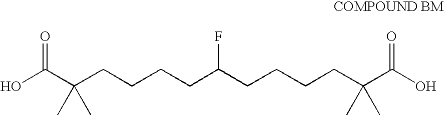

### 7-Fluoro-2,2,12,12-tetramethyl-tridecanedioic Acid

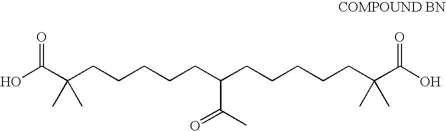

### 8-Acetyl-2,2,14,14-tetramethyl-pentadecanedioic Acid

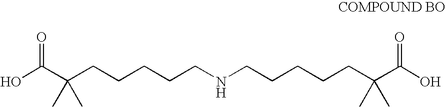

### 7-(6-Carboxy-6-methyl-heptylamino)-2,2-dimethyl-heptanoic Acid

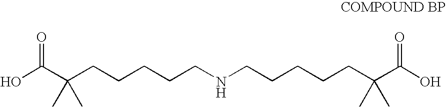

### 7-(6-Carboxy-6-methyl-heptylamino)-2,2-dimethyl-heptanoic Acid

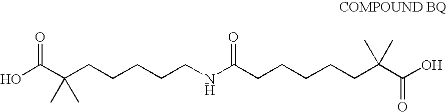

### 7-(7-Carboxy-7-methyl-octanoylamino)-2,2-dimethyl-heptanoic Acid

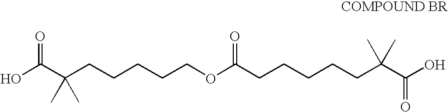

### 2,2-Dimethyl-octanedioic Acid 8-(6-carboxy-6-methyl-heptyl) Ester

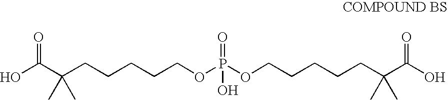

7-[(6-Carboxy-6-methyl-heptyloxy)-hydroxy-phosphoryloxy]-2,2-dimethyl-heptanoic Acid

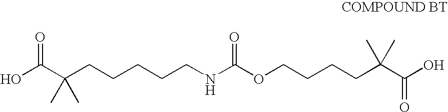

### 7-(5-Carboxy-5-methyl-hexyloxycarbonylamino)-2,2-dimethyl-heptanoic Acid

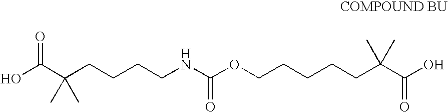

### 7-(5-Carboxy-5-methyl-hexylcarbamoyloxy)-2,2-dimethyl-heptanoic Acid

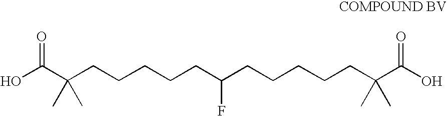

### 8-Fluoro-2,2,14,14-tetramethyl-pentadecanedioic Acid

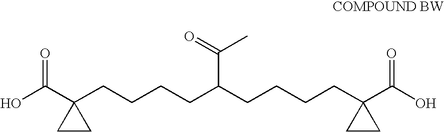

### 7-(1-Carboxy-cyclopropyl)-3-[4-(1-carboxy-cyclopropyl)-butyl]-heptan-2-one

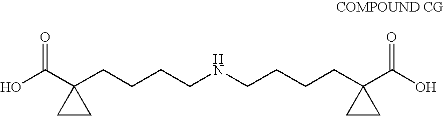

### (1-{4-[4-(1-Carboxy-cyclopropyl)-butylamino]-butyl]-cyclopropanoic Acid

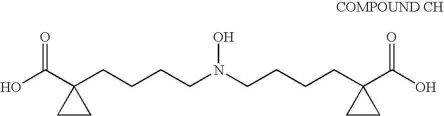

### 1-(4-{Hydroxy-[4-(1-carboxy-cyclopropyl)-butyl]-amino}-butyl)-cyclopropanoic Acid

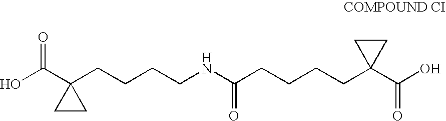

### 5-(1-Carboxyl-cyclopropyl)-pentanoic acid [4-(1-carboxy-cyclopropyl)-butyl]-amide

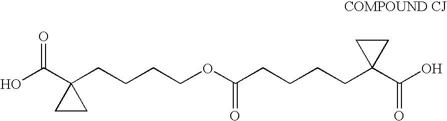

### 5-(1-Carboxy-cyclopropyl)-pentanoic Acid 4-(1-carboxy-cyclopropyl)-butyl Ester

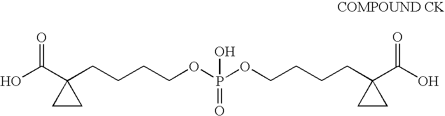

### Phosphoric Acid bis-[4-(1-carboxy-cyclopropyl)-butyl] Ester

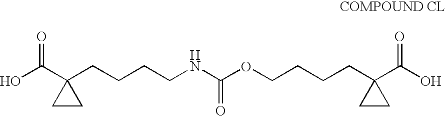

### [4-(1-Carboxy-cyclopropyl)-butyl]-carbamic acid 4-(1-carboxy-cyclopropyl)-butyl Ester

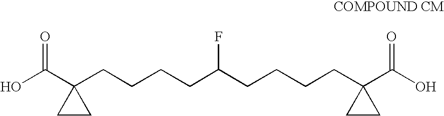

{1-[5-Fluoro-9-(1-carboxy-cyclopropyl)-nonyl]-cyclopropanoic Acid

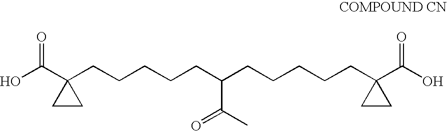

### 8-(1-Carboxy-cyclopropyl)-3-[5-(1-carboxy-cyclopropyl)-pentyl]-octan-2-one

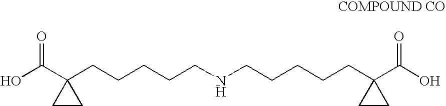

### (1-{5-[5-(1-Carboxy-cyclopropyl)-pentylamino]-pentyl}-cyclopropanoic Acid

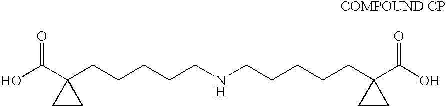

### (1-{5-[5-(1-Carboxy-cyclopropyl)-pentylamino]-pentyl}-cyclopropanoic Acid

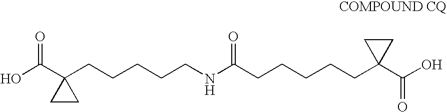

### 6-(1-Carboxy-cyclopropyl)-hexanoic acid [5-(1-hydroxymethyl-cyclopropyl)-pentyl]-amide

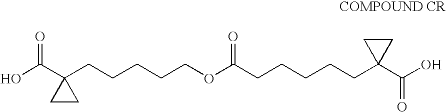

### 6-(1-Carboxy-cyclopropyl)-hexanoic acid 5-(1-carboxy-cyclopropyl)-pentyl Ester

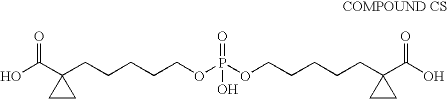

### Phosphoric Acid bis-[5-(1-carboxy-cyclopropyl)-pentyl] Ester

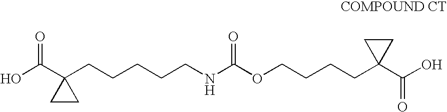

### [5-(1-Carboxy-cyclopropyl)-pentyl]-carbamic Acid 4-(1-carboxy-cyclopropyl)-butyl Ester

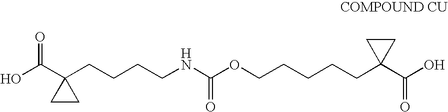

### [4-(1-Carboxy-cyclopropyl)-butyl]-carbamic acid 5-(1-carboxy-cyclopropyl)-pentyl Ester

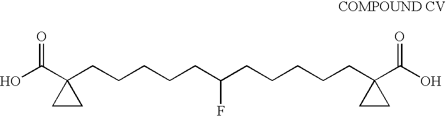

### {1-[6-Fluoro-11-(1-carboxy-cyclopropyl)-undecyl]-cyclopropanoic Acid

### Compound CW 5.4. Synthesis of the Compounds of Formula I

The compounds of the invention can be obtained via the synthetic methodology illustrated in Schemes 1-14. Starting materials useful for preparing the compounds of the invention and intermediates therefor are commercially available or can be prepared by well known synthetic methods.

Phosphorous derivatives of type I of this invention are prepared as described in Scheme 1, starting from compounds of type V. Alcohols of type V are prepared by methods already described in U.S. patent application Ser. Nos. 09/540,738, 09/976,899, 09/976,898, 09/976,867 and 09/976,938 the disclosures of which are incorporated herein by reference in their entirety, and in Larock Comprehensive Organic Transformations; Wiley-VCH: New York, 1999, incorporated herein by reference.

When Y¹ and Y² contain hydroxyl groups, they have to be protected prior to the reaction with phosphorous-introducing derivatives, by using selective methods for secondary alcohols as described by Greene et al., Protective Groups in Organic Synthesis, 3rd ed., John Wiley & Sons, Inc., (1999), incorporated herein by reference.

Phosphorous derivatives of type I of this invention are prepared following methods well documented in the chemical literature for the synthesis of mono and polyalkyl phosphates and polyphosphates, summarized in several reviews (J. B. Sweney, in Comprehensive Organic Functional Group Transformations, A. R. Katritzky, Meth-Cohn and C. W. Rees, Eds., Pergamon: Oxford, 1995, vol. 2, pp. 104-109 and Houben-Weyl, Methoden der Organische Chemie, Georg Thieme Verlag Stuttgart 1964, vol. XII/2), incorporated herein by reference.

Phosphoric acids (monoalkyl dihydrogenphosphates) of type I are prepared by treatment of the alcohol V with phosphorous oxychloride in an organic solvent, such as xylene or toluene, under heating in a temperature range of 100 to 150° C. for 2 to 24 hr, and subsequent hydrolysis of the phosphoric acid dichloride thus obtained, usually in the presence of an aqueous solution of sodium hydroxide.

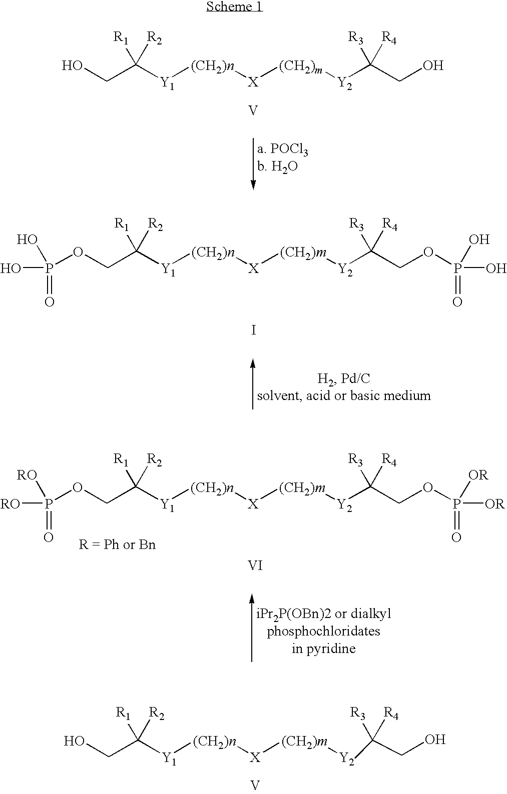

A general two-step method is the reaction of alcohols V with N,N-diisopropyl-dibenzylphophoramidite in the presence of tetrazole, followed by oxidation with MCPBA. In a typical procedure, alcohol V in a halogenated solvent, preferably dichloromethane, is treated with phosphoramidite in the presence of tetrazole for a period of one to ten hours, at temperatures ranging between −20 to 50° C. The benzyl phosphite thus obtained, MCPBA is added dropwise at −78° C., then the reaction mixture is stirred for an additional 2 to 8 hr. The benzyl phosphate is subjected to the usual workup, and then it is purified by flash chromatography on silicagel. The purified product undergoes hydrogenolysis in basic conditions (preferably sodium carbonate or bicarbonate) in the presence of palladium as a catalyst, using as a solvent mixtures of alcohols and water in various proportions, to produce the sodium salt of the phosphoric acid derivative. Alcohols used in mixtures are, but are not limited to, methanol, ethanol, propanol, n-butanol. t-butanol, preferably t-butanol. The free monoalkyl phosphate is prepared by treatment of the sodium salt with a dilute ice-cold solution of mineral acid.

Monoalkyl diphosphates (pyrophosphates) I′ and triphosphates I” are prepared as described in Scheme 2 from monoalkyl dihydrogenphosphates I by treatment with phosphoric acid and DCC.

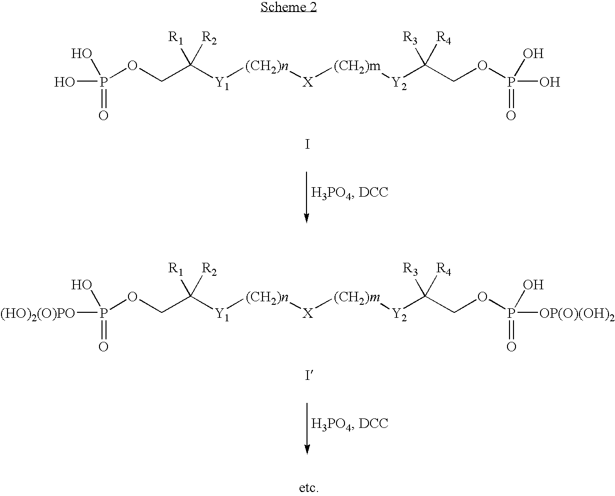

In a typical procedure, 1 mmol of monoalkyl dihydrogenphosphate and 800 mg 85% phosphoric acid are dissolved in a mixture of 2 ml water and 6 ml pyridine, and 4 g dicyclohexyl-carbodiimide are added. The mixture is stirred at room temperature for 5 hours and additional 2 g dicyclohexyl-carbodiimide and 2 ml pyridine are added. After 12 hours of vigorous stirring, 1 g dicyclohexyl-carbodiimide, 1 ml pyridine and 0.1 ml water are added. After a total of 26 hours, the precipitated dicyclohexylurea is filtered off and washed with 10 ml water. The filtrate is repeatedly extracted with diethyl ether and the ether is removed from the remaining aqueous layer in vacuo. The mercury (II)-salts of the mono-, pyro-, and triphosphoric acids are precipitated with Lohmann's reagent. These are suspended in water and decomposed with sulfuric acid at 0° C. The solution is neutralized with sodium hydroxide solution to pH=6 when the pyrophosphoric acid is separated by paper chromatography or by ion exchange through known methods. It is isolated as a barium salt by evaporating the neutralized filtrate at 0 to 5° C. and treatment with excess barium acetate.

A separation of the pyrophosphoric acid from the triphosphoric acid can also be done by precipitating the barium salt of the triphosphoric acid at pH=3.8. The filtrate is then adjusted to pH=8.5, which leads to precipitation of the barium salt of the pyrophosphoric acid.

Other preparation of di- and tri-phosphates are described in Scheme 3. Phosphoric acid ester-monoamides and phosphoric acid monoesters are reacted in pyridine in basic conditions to afford the diphosphates as salts (A. R. Todd et al. J. Chem. Soc. 1957, 1497). Monoalkyl diphosphates are also prepared by reacting monoalkyl dihydrogenphosphate with triethylammonium-N-butyl carbamyl phosphate, diimidazolyl-carbodiimide, or lithium phosphate, methods extensively reviewed in Houben-Weyl, Methoden der Organische Chemie, Georg Thieme Verlag Stuttgart 1964, vol. XII/2, pp. 143-210 and 872-879).

Monoalkyl triphosphates I″ are also prepared by treatment of diphosphates I′ with one equivalent of phosphoric acid, or by reacting the corresponding alcohols I with salicyl phosphorochloridite and pyrophosphate, followed by cleavage of the adduct thus obtained with iodine in pyridine (J. Ludwig, J. Org. Chem. 1989, 54, 631). In a typical experiment, salicyl phosphorochloridite is treated with alcohol V in an anhydrous solvent, such as pyridine, DMF, dioxane or mixtures of the solvents hereof, preferably pyridine/dioxane, and the well-stirred mixture is further reacted with a buffer solution of ammonium pyrophosphate in DMF and tri-n-butyl amine, to produce an intermediate that is oxidized with 1% iodine in pyridine/water to furnish the triphosphate. The product is isolated from the reaction mixture by methods well described in the reference procedure.

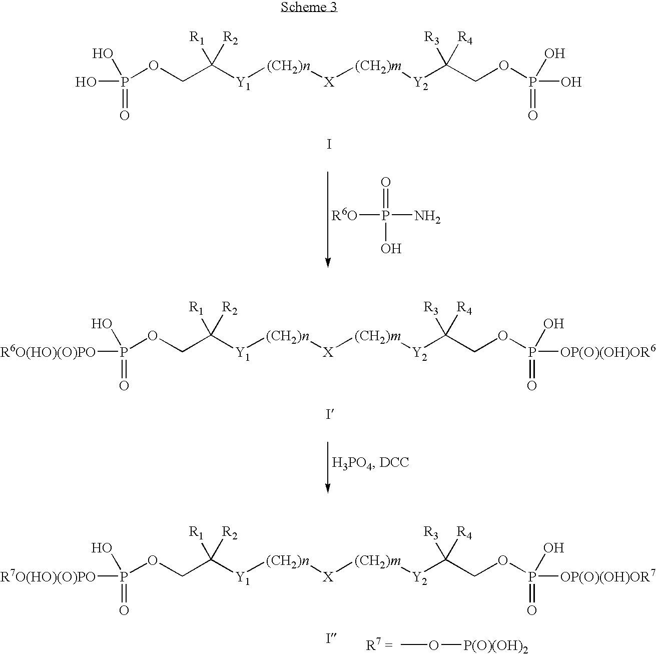

Monophosphates of formula I are coupled with nucleotides (commercially available, e.g. Sigma-Aldrich, or prepared by reacting a ribonucleotide with a purinic or pyrimidinic base by methods well described in the literature) to give nucleotide conjugates of compounds of type I. The reaction is performed by methods well described in the literature, as follows: (i) treating the phosphate and a nucleotide in the presence of an amine in a multistep reaction as described by Givens, R. S. et al. Tetrahedron Lett. 1996, 37, 6259-6262; Bhattacharya, A. K. et al. Bioorg. Med. Chem. 2002, 10, 1129-1136; (ii) treating the phosphate and the nucleotide with 1,1′-carbonyldiimidazole in an ammonium buffer as described by Hampton, A. et al. J. Med. Chem. 1982, 25, 801-805; Hong, C. I. et al. J. Med. Chem. 1986, 29, 2038-2044; Hong, C. I. et al. J. Med. Chem. 1990, 33, 1380-1386; (iii) treating the nucleotide with a phosphoric ester morpholin-4-yl amide of formula I in the presence of tetrazole as described in Peng, Z. -H. et al. Org. Lett. 2002, 4, 161-164; Ichikawa, Y. et al. J. Org. Chem. 1992, 57, 2943-2946; Adelhost, K. et al. Carbohydrate Res. 1993, 242, 69-76; (iv) treating a p-tolyl ester of a thiophosphoric acid derivative of compounds I with a nucleotide in an ammonium buffer, as described in Noort, D. et al. Recl. Trav. Chim. Pays-Bas 1991, 110, 53-56.

In a typical procedure, 3′-O-methylguanosine is phosphorylated with POCl₃/PO(Me)₃ at temperatures between −10 and 5° C., followed by treatment with MeI to give selectively the N7-methylated pyridinium salt. Then, the tributylammonium salt of the of the 3′-O-Me-m7GMP was further phosphorylated to dimethylated GDP with tributylammonium orthophosphate in dimethylformamide in the presence of carbonyldiimidazole. The activated morpholidate of the phosphate of type I is added in the presence of tetrazole in DMSO and the mixture is stirred at room temperature for 72 hr to a week, until the reaction is deemed complete. The nucleotide conjugate can be separated by usual methods, preferably by DEAE-Sephadex A25 Chromatography and couterion exchange. Scheme 4 illustrates the synthesis of compounds of formula I when coupled with nucleotides.

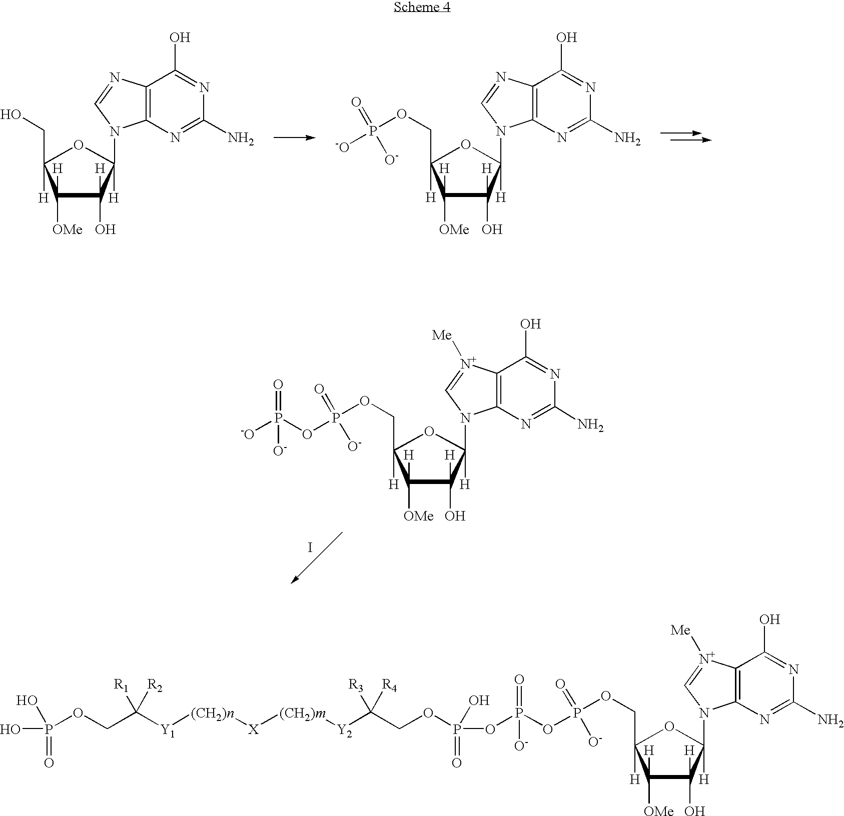

Scheme 5 illustrates the synthesis of amines XII from aldehydes XIV via the imine XV (see Wang et al. J. Org. Chem. 1995, 60, 7364, Tanaka et al. J. Med. Chem. 1998, 41, 2390, Smith and March, Advanced Organic Chemistry: Reactions, Mechanisms and Structures, 5th Ed.; Wiley: New York, 2001; p 1203, and references cited herein, and methods referenced in Larock, Comprehensive Organic Transformations, 2nd Ed., Wiley: New York 1999, p. 835). In a typical procedure, a mixture of aldehyde and ammonium formate or ammonium oxalate is heated at temperatures higher than 120° C., preferably at 140° C., until no more water is distilled off. Then the temperature of the reaction mixture is raised to over 150° C., preferably 180-200° C., for 2 to 10 hours. The reaction mixture is cooled at room temperature, treated with concentrated HCl at room temperature or higher for 2 to 6 hours, and the organic impurities extracted with an organic solvent such as diethyl-ether, t-butyl methyl ether, benzene, toluene, hexane, preferably toluene. Afterwards, the aqueous layer is made alkaline with an aqueous sodium hydroxide solution and the amine is extracted in an organic solvent and purified by methods commonly used in the field. Amines XII are also prepared from a halide XVI (X=Hal) and dibenzylamine. In a typical procedure, halide XVI is treated with dibenzylamine neat at temperatures in the range of 100 to 150° C., preferably 130° C., or in diglyme in the presence of potassium carbonate at temperatures in the range of 120 to 180° C., preferably at 140° C., until no more change in the starting material is observed by an analytical method such as but not limited to High Pressure Liquid Chromatography or Thin Layer Chromatography. When the reaction is complete, the amine is converted into a hydrochloride and is precipitated as a hydrochloride in a dry solvent such as 2-propanol. The dibenzylamine derivative XVII is treated with 10% Pd/C and ammonium formate in methanol at reflux for 2 to 24 hours, then filtration through Celite; evaporation of the solvent yields the crude amine XII, which is purified by usual methods (Purchase et al. J. Org. Chem. 1991, 56, 457-459).

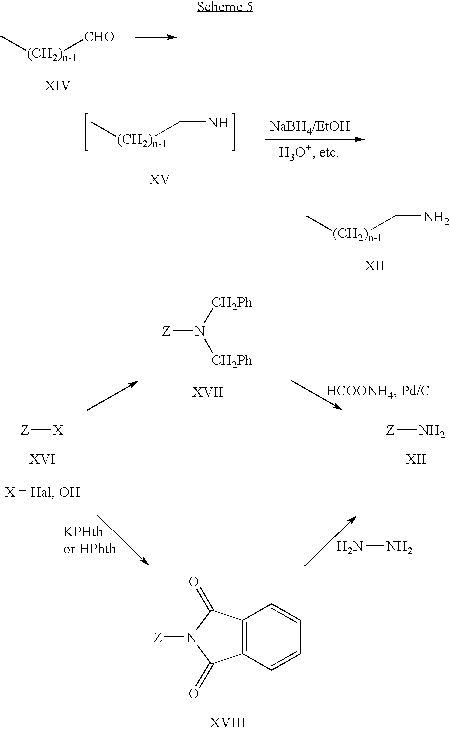

Scheme 5 also illustrates the preparation of amines of formula XII by Gabriel synthesis starting from halo-derivatives XVI (for general references see Gibson et al. Angew. Chem. 1968, 80, 986, Macholan, L. Coll. Czech. Chem. Comm. 1974, 39, 653-661 Smith and March, Advanced Organic Chemistry: Reactions, Mechanisms and Structures, 5th Ed.; Wiley: New York, 2001; p 513, and references cited herein). For an improved Gabriel synthesis, see also Sheehan et al. J. Amer. Chem. Soc. 1950, 72, 2786-2788. In a typical procedure, bromide XVI (X=Br) and potassium phthalimide in DMF are kept at room temperature or heated to 90° C. for 0.5 to 4 hours, extracted in a solvent, or precipitated by addition of water and recrystallized. The phthalimide of formula XVIII thus obtained is treated in methanol with an 85% aqueous solution of hydrazine hydrate for 15 min to one hour. Addition of water and removal of the methanol is followed by addition of HCl and heating under reflux for 1 hour, removal of crystalline phthalhydrazide by cooling to 0° C., then workup of the amine XII from the filtrate. In an alternative procedure potassium phthalimide and potassium carbonate in the presence of catalytic amounts of benzyltriethylammonium chloride in acetone are refluxed for 40 min, then bromide of formula XVI is added dropwise for 4 hr at reflux. When the reaction is complete, the mixture is subjected to separation and purification by known methods, such as chromatography or recrystallization. As a reference see Sasse et al. J. Med. Chem. 2001, 44, 694-702 and Khan J. Org. Chem. 1996, 61, 8063-8068. The reactions described above are all monitored by an analytical method such as HPLC, tlc or NMR. N-Alkylphthalimides of formula XVIII are also prepared starting from an alcohol and phthalimide in Mitsunobu conditions (Mitsunobu et al. J. Amer. Chem. Soc. 1972, 94, 679-680). In a typical procedure, an alcohol of formula XVI (X—OH) is treated with phthalimide in the presence of triphenylphosphine and diethyl azodicarboxylate in dry THF at 0° C., then the mixture is stirred overnight at room temperature. After evaporation of the solvent, the phthalimide is separated and purified in the usual manner. Subsequently, the phthalimide in ethanol is treated with hydrazine hydrate at reflux for 15 min, and then the suspension cooled, acidified and filtered. The amine of formula XII is recovered from the filtrate as a hydrochloride or as a free base by usual separation methods.

The synthesis of 6-(5,5-dimethyl-6-hydroxy-hexyl amino)-2,2-dimethyl-hexan-1-ol hydrochloride (H) is shown in Scheme 6. The sequence starts with a phase-transfer catalyzed reaction between key building block XIX [Brown, G. R. et al. J Med. Chem. 1995, 38, 1615] and p-toluenesulfonamide [Isele, G. et al. Synthesis 1981, 455-457] to give the protected amine intermediate XX2 in very good yield (96%, calculated for the crude product). Since both THP- and Ts-protective groups are cleavable by strong acids, one-step deprotection of XX to the desired amine H is possible with 30% HBr in acetic acid with phenol as scavenger at rt [Haskell, B. E. et al. J. Org. Chem. 1976, 41, 159-160; Bergeron, R. J. et al. J. Med. Chem. 1997, 40, 1475-1494]. Also, aqueous 48% HBr and phenol [Compagnone, R. S. et al. J. Org. Chem. 1986, 51, 1713-1710] at reflux temperature could be used.

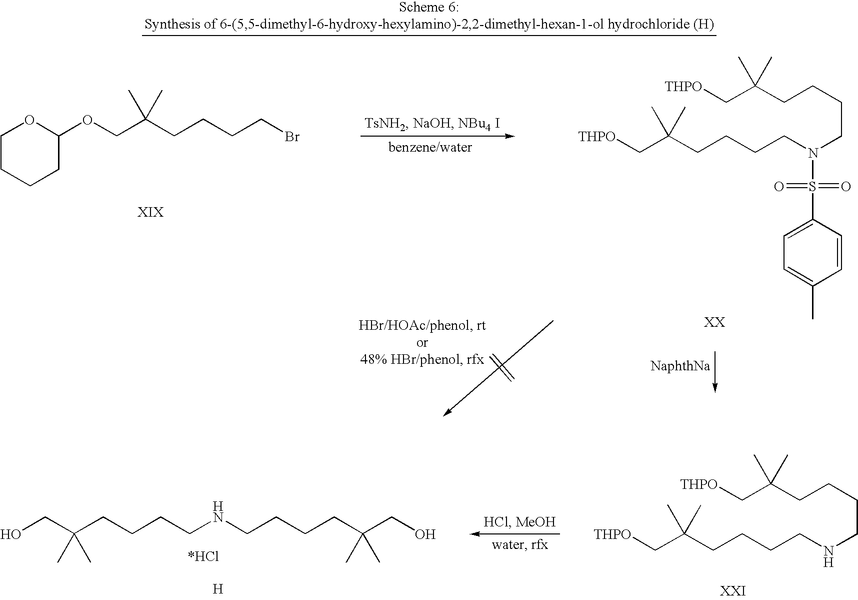

A two-step deprotection sequence usually gives higher yields. In the first step, the tosylamide protection was removed using sodium naphthalenide in dimethoxyethane at −78° C. [Bergeron, R. J. et al. J. Med. Chem. 2000, 43, 224-235] to furnish the crude product XXI, which is subsequently deprotected by treatment with concentrated HCl in methanol. The target compound H is finally obtained as a reddish glass, in a 40% yield calculated over two steps.

The synthesis of phosphoric acid bis(5,5-dimethyl-6-hydroxy-hexyl)ester (L) can be performed by using two different strategies (Scheme 7). Bromide XIX is reacted with tetramethylamminium phosphate in analogy to a method designed for the synthesis of mixed dialkyl phosphates [Baumann, R. A. Synthesis 1974, 870-872].

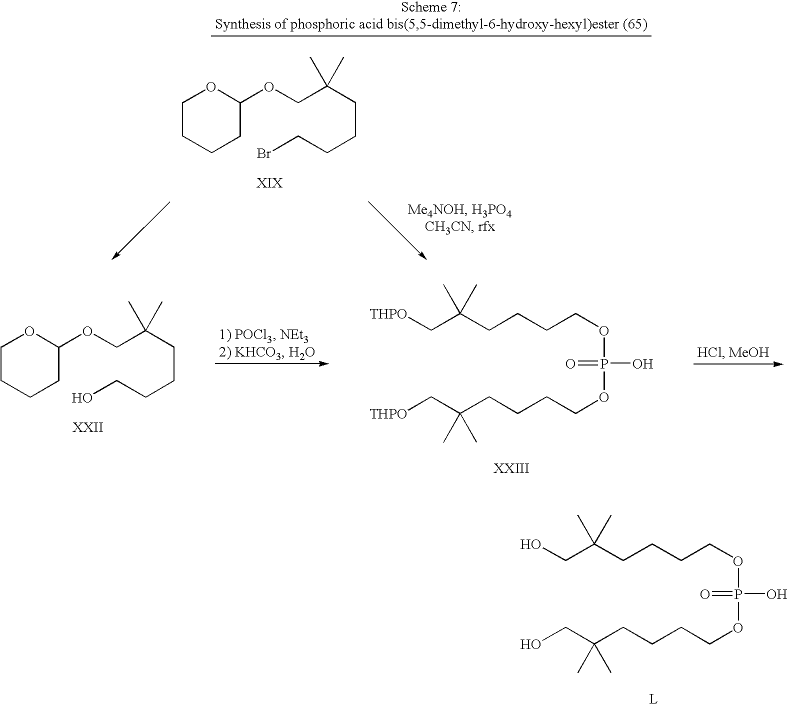

A second strategy departs from alcohol XXII (prepared from XIX by hydrolysis with K₂CO₃ in water/DMSO). This compound was reacted with suitable phosphoric acid derivatives, such as the reaction of XXII with phosphoric acid, triethylamine, and trichloroacetonitrile as condensing agent at 90° C. [Methoden der Organischen Chemie (Houben-Weyl), Bd. XII/2, 1964, 232]. Synthesis of XXII can be accomplished by treatment of alcohol XXII with phosphorous oxychloride and triethylamine in diethyl ether [Moss, R. A. et al. Tetrahedron Lett. 2000, 41, 3275-3278]. The THP-protection in XXIII is subsequently removed (methanol-concd. HCl at reflux) to furnish the dialkyl phosphate L (9% calculated from alcohol XIX) as a viscous oil.

The preparation of 6-[hydroxy-(6-hydroxy-5,5-dimethyl-hexyl)-amino]-2,2-dimethyl-hexan-1-ol is shown in Scheme 8.

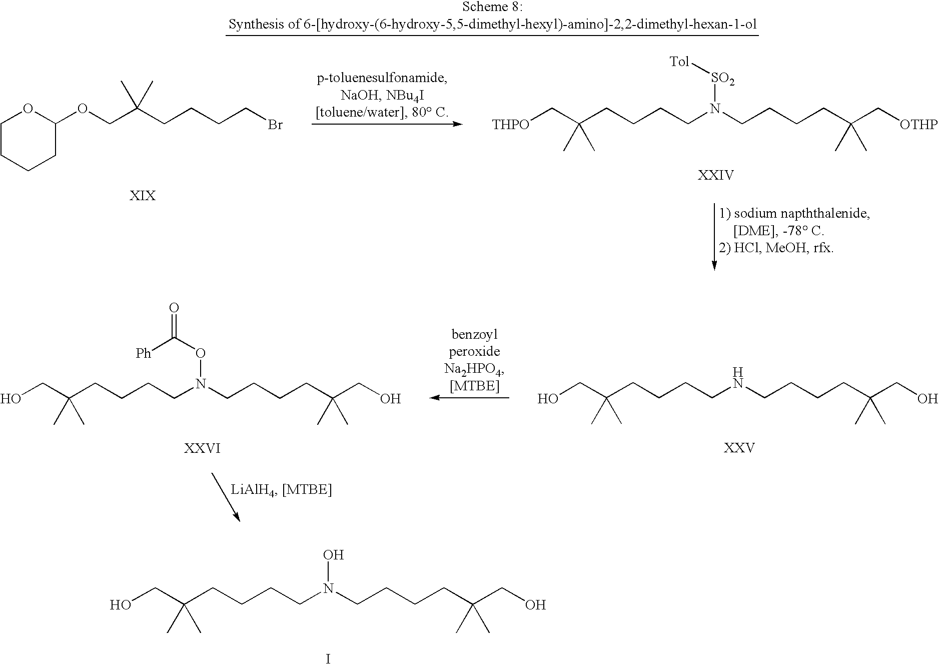

The tosylamide XXIV is prepared by heating a mixture of bromide, p-toluenesulfonamide, sodium hydroxide, and tetra-n-butylammonium iodide in a toluene/water mixture for 20 h at 80° C. [Isele, G. et al. Synthesis 1981, 455-457]. The product can be used without further purification, or purified by chromatographic methods.

Compound XXIV is then reacted with sodium naphthalenide in anhydrous dimethoxyethane at −78° C. to remove the tosyl group. The THP-protected intermediate is subsequently hydrolyzed with concentrated HCl in methanol at reflux for 2 h [Bergeron, R. J. et al. J. Med. Chem. 2000, 43, 224-235] to give the free amine XXV, which is further converted to the N-benzoyloxy intermediate XXVI by reaction with benzoyl peroxide and sodium hydrogen phosphate in t-butylmethyl ether at 45° C. for 20 h [Biloski, A. J. et al. Synthesis 1983, 537-538]. Purification by column chromatography (silica, hexanes/ethyl acetate=3:1 to 1:1} gives XXVI, which is treated with lithium aluminum hydride in anhydrous MTBE at rt for 2 h [Beckett, A. H et al. Tetrahedron 1973, 29, 4189-4193] to give the crude product I in a mixture with benzyl alcohol (ca. 27%). This crude product was crystallized (from heptane/MTBE/CH₂Cl₂=30/30/15 ml) to give the target cohydroxylamine compound I as a white solid.

### 5.5. Therapeutic Uses of Compounds of the Invention

In accordance with the invention, the compounds of formula I or a pharmaceutically acceptable salt thereof or an acyl coenzyme-A mimic identified by a method disclosed herein (collectively, “the compounds of the invention”) are useful for administration to a patient, preferably a human, with or at risk of cardiovascular disease, a dyslipidemia, a dyslipoproteinemia, a disorder of glucose metabolism, Alzheimer's Disease, Syndrome X, a PPAR-associated disorder, septicemia, a thrombotic disorder, obesity, pancreatitis, hypertension, a renal disease, cancer, inflammation, bacterial infection or impotence. In one embodiment, “treatment” or “treating” refers to an amelioration of a disease or disorder, or at least one discernible symptom thereof. In another embodiment, “treatment” or “treating” refers to delaying the onset of a disease or disorder or inhibiting the progression thereof, either physically, e.g., stabilization of a discernible symptom, physiologically, e.g., stabilization of a physical parameter, or both.

In certain embodiments, the compounds of the invention or the compositions of the invention are administered to a patient, preferably a human, as a preventative measure against such diseases. As used herein, “prevention” or “preventing” refers to a reduction of the risk of acquiring a given disease or disorder. In a preferred mode of the embodiment, the compositions of the present invention are administered as a preventative measure to a patient, preferably a human having a genetic predisposition to a cardiovascular disease, a dyslipidemia, a dyslipoproteinemia, a disorder of glucose metabolism, Alzheimer's Disease, Syndrome X, a PPAR-associated disorder, septicemia, a thrombotic disorder, obesity, pancreatitis, hypertension, a renal disease, cancer, inflammation, bacterial infection or impotence. Examples of such genetic predispositions include but are not limited to the ε4 allele of apolipoprotein E, which increases the likelihood of Alzheimer's Disease; a loss of function or null mutation in the lipoprotein lipase gene coding region or promoter (e.g., mutations in the coding regions resulting in the substitutions D9N and N291S; for a review of genetic mutations in the lipoprotein lipase gene that increase the risk of cardiovascular diseases, dyslipidemias and dyslipoproteinemias, see Hayden and Ma, 1992, Mol. Cell Biochem. 113:171-176); and familial combined hyperlipidemia and familial hypercholesterolemia.

In another preferred mode of the embodiment, the compounds of the invention or compositions of the invention are administered as a preventative measure to a patient having a non-genetic predisposition to a cardiovascular disease, a dyslipidemia, a dyslipoproteinemia, a disorder of glucose metabolism, Alzheimer's Disease, Syndrome X, a PPAR-associated disorder, septicemia, a thrombotic disorder, obesity, pancreatitis, hypertension, a renal disease, cancer, inflammation, bacterial infection or impotence. Examples of such non-genetic predispositions include but are not limited to cardiac bypass surgery and percutaneous transluminal coronary angioplasty, which often lead to restenosis, an accelerated form of atherosclerosis; diabetes in women, which often leads to polycystic ovarian disease; and cardiovascular disease, which often leads to impotence. Accordingly, the compositions of the invention may be used for the prevention of one disease or disorder and concurrently treating another (e.g., prevention of polycystic ovarian disease while treating diabetes; prevention of impotence while treating a cardiovascular disease).

### 5.5.1. Cardiovascular Diseases for Treatment or Prevention

The present invention provides methods for the treatment or prevention of a cardiovascular disease, comprising administering to a patient a therapeutically effective amount of a compound or a composition comprising a compound of the invention and a pharmaceutically acceptable vehicle. As used herein, the term “cardiovascular diseases” refers to diseases of the heart and circulatory system. These diseases are often associated with dyslipoproteinemias and/or dyslipidemias. Cardiovascular diseases which the compositions of the present invention are useful for preventing or treating include but are not limited to arteriosclerosis; atherosclerosis; stroke; ischemia; endothelium dysfunctions, in particular those dysfunctions affecting blood vessel elasticity; peripheral vascular disease; coronary heart disease; myocardial infarcation; cerebral infarction and restenosis.

### 5.5.2. Dyslipidemias for Treatment or Prevention

The present invention provides methods for the treatment or prevention of a dyslipidemia comprising administering to a patient a therapeutically effective amount of a compound or a composition comprising a compound of the invention and a pharmaceutically acceptable vehicle.

As used herein, the term “dyslipidemias” refers to disorders that lead to or are manifested by aberrant levels of circulating lipids. To the extent that levels of lipids in the blood are too high, the compositions of the invention are administered to a patient to restore normal levels. Normal levels of lipids are reported in medical treatises known to those of skill in the art. For example, recommended blood levels of LDL, HDL, free triglycerides and others parameters relating to lipid metabolism can be found at the web site of the American Heart Association and that of the National Cholesterol Education Program of the National Heart, Lung and Blood Institute (http://www.americanheart.org/cholesterol/about_level.html and http://www.nhlbi.nih.gov/health/public/heart/chol/hbc_what.html, respectively). At the present time, the recommended level of HDL cholesterol in the blood is above 35 mg/dL; the recommended level of LDL cholesterol in the blood is below 130 mg/dL; the recommended LDL:HDL cholesterol ratio in the blood is below 5:1, ideally 3.5:1; and the recommended level of free triglycerides in the blood is less than 200 mg/dL.

Dyslipidemias which the compositions of the present invention are useful for preventing or treating include but are not limited to hyperlipidemia and low blood levels of high density lipoprotein (HDL) cholesterol. In certain embodiments, the hyperlipidemia for prevention or treatment by the compounds of the present invention is familial hypercholesterolemia; familial combined hyperlipidemia; reduced or deficient lipoprotein lipase levels or activity, including reductions or deficiencies resulting from lipoprotein lipase mutations; hypertriglyceridemia; hypercholesterolemia; high blood levels of ketone bodies (e.g. β-OH butyric acid); high blood levels of Lp(a) cholesterol; high blood levels of low density lipoprotein (LDL) cholesterol; high blood levels of very low density lipoprotein (VLDL) cholesterol and high blood levels of non-esterified fatty acids.

The present invention further provides methods for altering lipid metabolism in a patient, e.g., reducing LDL in the blood of a patient, reducing free triglycerides in the blood of a patient, increasing the ratio of HDL to LDL in the blood of a patient, and inhibiting saponified and/or non-saponified fatty acid synthesis, said methods comprising administering to the patient a compound or a composition comprising a compound of the invention in an amount effective alter lipid metabolism.

5.5.3. Dyslipoproteinemias for Treatment or Prevention

The present invention provides methods for the treatment or prevention of a dyslipoproteinemia comprising administering to a patient a therapeutically effective amount of a compound or a composition comprising a compound of the invention and a pharmaceutically acceptable vehicle.

As used herein, the term “dyslipoproteinemias” refers to disorders that lead to or are manifested by aberrant levels of circulating lipoproteins. To the extent that levels of lipoproteins in the blood are too high, the compositions of the invention are administered to a patient to restore normal levels. Conversely, to the extent that levels of lipoproteins in the blood are too low, the compositions of the invention are administered to a patient to restore normal levels. Normal levels of lipoproteins are reported in medical treatises known to those of skill in the art.

Dyslipoproteinemias which the compositions of the present invention are useful for preventing or treating include but are not limited to high blood levels of LDL; high blood levels of apolipoprotein B (apo B); high blood levels of Lp(a); high blood levels of apo(a); high blood levels of VLDL; low blood levels of HDL; reduced or deficient lipoprotein lipase levels or activity, including reductions or deficiencies resulting from lipoprotein lipase mutations; hypoalphalipoproteinemia; lipoprotein abnormalities associated with diabetes; lipoprotein abnormalities associated with obesity; lipoprotein abnormalities associated with Alzheimer's Disease; and familial combined hyperlipidemia.

The present invention further provides methods for reducing apo C-II levels in the blood of a patient; reducing apo C-III levels in the blood of a patient; elevating the levels of HDL associated proteins, including but not limited to apo A-I, apo A-11, apo A-IV and apo E in the blood of a patient; elevating the levels of apo E in the blood of a patient, and promoting clearance of triglycerides from the blood of a patient, said methods comprising administering to the patient a compound or a composition comprising a compound of the invention in an amount effective to bring about said reduction, elevation or promotion, respectively.

### 5.5.4. Glucose Metabolism Disorders for Treatment or Prevention

The present invention provides methods for the treatment or prevention of a glucose metabolism disorder, comprising administering to a patient a therapeutically effective amount of a compound or a composition comprising a compound of the invention and a pharmaceutically acceptable vehicle. As used herein, the term “glucose metabolism disorders” refers to disorders that lead to or are manifested by aberrant glucose storage and/or utilization. To the extent that indicia of glucose metabolism (i.e., blood insulin, blood glucose) are too high, the compositions of the invention are administered to a patient to restore normal levels. Conversely, to the extent that indicia of glucose metabolism are too low, the compositions of the invention are administered to a patient to restore normal levels. Normal indicia of glucose metabolism are reported in medical treatises known to those of skill in the art.

Glucose metabolism disorders which the compositions of the present invention are useful for preventing or treating include but are not limited to impaired glucose tolerance; insulin resistance; insulin resistance related breast, colon or prostate cancer; diabetes, including but not limited to non-insulin dependent diabetes mellitus (NIDDM), insulin dependent diabetes mellitus (IDDM), gestational diabetes mellitus (GDM), and maturity onset diabetes of the young (MODY); pancreatitis; hypertension; polycystic ovarian disease; and high levels of blood insulin and/or glucose.

The present invention further provides methods for altering glucose metabolism in a patient, for example to increase insulin sensitivity and/or oxygen consumption of a patient, said methods comprising administering to the patient a compound or a composition comprising a compound of the invention in an amount effective to alter glucose metabolism.

### 5.5.5. PPAR Associated Disorders for Treatment or Prevention

The present invention provides methods for the treatment or prevention of a PPAR-associated disorder, comprising administering to a patient a therapeutically effective amount of a compound or a composition comprising a compound of the invention and a pharmaceutically acceptable vehicle. As used herein, “treatment or prevention of PPAR associated disorders” encompasses treatment or prevention of rheumatoid arthritis; multiple sclerosis; psoriasis; inflammatory bowel diseases; breast; colon or prostate cancer; low levels of blood HDL; low levels of blood, lymph and/or cerebrospinal fluid apo E; low blood, lymph and/or cerebrospinal fluid levels of apo A-I; high levels of blood VLDL; high levels of blood LDL; high levels of blood triglyceride; high levels of blood apo B; high levels of blood apo C-III and reduced ratio of post-heparin hepatic lipase to lipoprotein lipase activity. HDL may be elevated in lymph and/or cerebral fluid.

### 5.5.6. Renal Diseases for Treatment or Prevention

The present invention provides methods for the treatment or prevention of a renal disease, comprising administering to a patient a therapeutically effective amount of a compound or a composition comprising a compound of the invention and a pharmaceutically acceptable vehicle. Renal diseases that can be treated by the compounds of the present invention include glomerular diseases (including but not limited to acute and chronic glomerulonephritis, rapidly progressive glomerulonephritis, nephrotic syndrome, focal proliferative glomerulonephritis, glomerular lesions associated with systemic disease, such as systemic lupus erythematosus, Goodpasture's syndrome, multiple myeloma, diabetes, neoplasia, sickle cell disease, and chronic inflammatory diseases), tubular diseases (including but not limited to acute tubular necrosis and acute renal failure, polycystic renal diseasemedullary sponge kidney, medullary cystic disease, nephrogenic diabetes, and renal tubular acidosis), tubulointerstitial diseases (including but not limited to pyelonephritis, drug and toxin induced tubulointerstitial nephritis, hypercalcemic nephropathy, and hypokalemic nephropathy) acute and rapidly progressive renal failure, chronic renal failure, nephrolithiasis, or tumors (including but not limited to renal cell carcinoma and nephroblastoma). In a most preferred embodiment, renal diseases that are treated by the compounds of the present invention are vascular diseases, including but not limited to hypertension, nephrosclerosis, microangiopathic hemolytic anemia, atheroembolic renal disease, diffuse cortical necrosis, and renal infarcts.

### 5.5.7. Cancers for Treatment or Prevention

The present invention provides methods for the treatment or prevention of cancer, comprising administering to a patient a therapeutically effective amount of a compound or a composition comprising a compound of the invention and a pharmaceutically acceptable vehicle. Cancers that can be treated or prevented by administering the compounds or the compositions of the invention include, but are not limited to, human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenström's macroglobulinemia, and heavy chain disease. In a most preferred embodiment, cancers that are treated or prevented by administering the compounds of the present invention are insulin resistance or Syndrome X related cancers, including but not limited to breast, prostate and colon cancer.

### 5.5.8. Other Diseases for Treatment or Prevention

The present invention provides methods for the treatment or prevention of Alzheimer's Disease, Syndrome X, septicemia, thrombotic disorders, obesity, pancreatitis, hypertension, inflammation, bacterial infection multiple sclerosis, impotence and multiple sclerosis, comprising administering to a patient a therapeutically effective amount of a compound or a composition comprising a compound of the invention and a pharmaceutically acceptable vehicle.

As used herein, “treatment or prevention of Alzheimer's Disease” encompasses treatment or prevention of lipoprotein abnormalities associated with Alzheimer's Disease.

As used herein, “treatment or prevention of Syndrome X or Metabolic Syndrome” encompasses treatment or prevention of a symptom thereof, including but not limited to impaired glucose tolerance, hypertension and dyslipidemia/dyslipoproteinemia.

As used herein, “treatment or prevention of septicemia” encompasses treatment or prevention of septic shock.

As used herein, “treatment or prevention of thrombotic disorders” encompasses treatment or prevention of high blood levels of fibrinogen and promotion of fibrinolysis.

In addition to treating or preventing obesity, the compositions of the invention can be administered to an individual to promote weight reduction of the individual.

### 5.6. Surgical Uses

Cardiovascular diseases such as atherosclerosis often require surgical procedures such as angioplasty. Angioplasty is often accompanied by the placement of a reinforcing a metallic tube-shaped structure known as a “stent” into a damaged coronary artery. For more serious conditions, open heart surgery such as coronary bypass surgery may be required. These surgical procedures entail using invasive surgical devices and/or implants, and are associated with a high risk of restenosis and thrombosis. Accordingly, the compounds and compositions of the invention may be used as coatings on surgical devices (e.g., catheters) and implants (e.g., stents) to reduce the risk of restenosis and thrombosis associated with invasive procedures used in the treatment of cardiovascular diseases.

### 5.7. Veterinary and Livestock Uses

A composition of the invention can be administered to a non-human animal for a veterinary use for treating or preventing a disease or disorder disclosed herein.

In a specific embodiment, the non-human animal is a household pet. In another specific embodiment, the non-human animal is a livestock animal. In a preferred embodiment, the non-human animal is a mammal, most preferably a cow, horse, sheep, pig, cat, dog, mouse, rat, rabbit, or guinea pig. In another preferred embodiment, the non-human animal is a fowl species, most preferably a chicken, turkey, duck, goose, or quail.

In addition to veterinary uses, the compounds and compositions of the invention can be used to reduce the fat content of livestock to produce leaner meats. Alternatively, the compounds and compositions of the invention can be used to reduce the cholesterol content of eggs by administering the compounds to a chicken, quail, or duck hen. For non-human animal uses, the compounds and compositions of the invention can be administered via the animals' feed or orally as a drench composition.

### 5.8. Therapeutic/Prophylactic Administration and Compositions

Due to the activity of the compounds and compositions of the invention, they are useful in veterinary and human medicine. As described above, the compounds and compositions of the invention are useful for the treatment or prevention of cardiovascular diseases, dyslipidemias, dyslipoproteinemias, glucose metabolism disorders, Alzheimer's Disease, Syndrome X, PPAR-associated disorders, septicemia, thrombotic disorders, obesity, pancreatitis, hypertension, renal disease, cancer, inflammation, bacterial infection and impotence.

The invention provides methods of treatment and prophylaxis by administration to a patient of a therapeutically effective amount of a compound or a composition comprising a compound of the invention. The patient is an animal, including, but not limited, to an animal such a cow, horse, sheep, pig, chicken, turkey, quail, cat, dog, mouse, rat, rabbit, guinea pig, etc., and is more preferably a mammal, and most preferably a human.

The compounds and compositions of the invention, are preferably administered orally. The compounds and compositions of the invention may also be administered by any other convenient route, for example, by intravenous infusion or bolus injection, by absorption through epithelial or mucocutaneous linings (e.g., oral mucosa, rectal and intestinal mucosa, etc.) and may be administered together with another biologically active agent. Administration can be systemic or local. Various delivery systems are known, e.g., encapsulation in liposomes, microparticles, microcapsules, capsules, etc., and can be used to administer a compound of the invention. In certain embodiments, more than one compound of the invention is administered to a patient. Methods of administration include but are not limited to intradermal, intramuscular, intraperitoneal, intravenous, subcutaneous, intranasal, epidural, oral, sublingual, intranasal, intracerebral, intravaginal, transdermal, rectally, by inhalation, or topically, particularly to the ears, nose, eyes, or skin. The preferred mode of administration is left to the discretion of the practitioner, and will depend in-part upon the site of the medical condition. In most instances, administration will result in the release of the compounds of the invention into the bloodstream.

In specific embodiments, it may be desirable to administer one or more compounds of the invention locally to the area in need of treatment. This may be achieved, for example, and not by way of limitation, by local infusion during surgery, topical application, e.g., in conjunction with a wound dressing after surgery, by injection, by means of a catheter, by means of a suppository, or by means of an implant, said implant being of a porous, nonporous, or gelatinous material, including membranes, such as sialastic membranes, or fibers. In one embodiment, administration can be by direct injection at the site (or former site) of an atherosclerotic plaque tissue.

In certain embodiments, for example, for the treatment of Alzheimer's Disease, it may be desirable to introduce one or more compounds of the invention into the central nervous system by any suitable route, including intraventricular, intrathecal and epidural injection. Intraventricular injection may be facilitated by an intraventricular catheter, for example, attached to a reservoir, such as an Ommaya reservoir.

Pulmonary administration can also be employed, e.g., by use of an inhaler or nebulizer, and formulation with an aerosolizing agent, or via perfusion in a fluorocarbon or synthetic pulmonary surfactant. In certain embodiments, the compounds of the invention can be formulated as a suppository, with traditional binders and vehicles such as triglycerides.

In another embodiment, the compounds and compositions of the invention can be delivered in a vesicle, in particular a liposome (see Langer, 1990, Science 249:1527-1533; Treat et al., in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.).

In yet another embodiment, the compounds and compositions of the invention can be delivered in a controlled release system. In one embodiment, a pump may be used (see Langer, supra; Sefton, 1987, CRC Crit. Ref. Biomed. Eng. 14:201; Buchwald et al., 1980, Surgery 88:507 Saudek et al., 1989, N. Engl. J Med. 321:574). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, 1983, J. Macromol. Sci. Rev. Macromol. Chem. 23:61; see also Levy et al., 1985, Science 228:190; During et al., 1989, Ann. Neurol. 25:351; Howard et al., 1989, J. Neurosurg. 71:105). In yet another embodiment, a controlled-release system can be placed in proximity of the target area to be treated, e.g., the liver, thus requiring only a fraction of the systemic dose (see, e.g., Goodson, in Medical Applications of Controlled Release, supra, vol. 2, pp. 115-138 (1984)). Other controlled-release systems discussed in the review by Langer, 1990, Science 249:1527-1533) maybe used.

The present compositions will contain a therapeutically effective amount of a compound of the invention, optionally more than one compound of the invention, preferably in purified form, together with a suitable amount of a pharmaceutically acceptable vehicle so as to provide the form for proper administration to the patient.

In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “vehicle” refers to a diluent, adjuvant, excipient, or carrier with which a compound of the invention is administered. Such pharmaceutical vehicles can be liquids, such as water and oils, including those of petroleum, animal, vegetable or synthetic origin, such as peanut oil, soybean oil, mineral oil, sesame oil and the like. The pharmaceutical vehicles can be saline, gum acacia, gelatin, starch paste, talc, keratin, colloidal silica, urea, and the like. In addition, auxiliary, stabilizing, thickening, lubricating and coloring agents may be used. When administered to a patient, the compounds and compositions of the invention and pharmaceutically acceptable vehicles are preferably sterile. Water is a preferred vehicle when the compound of the invention is administered intravenously. Saline solutions and aqueous dextrose and glycerol solutions can also be employed as liquid vehicles, particularly for injectable solutions. Suitable pharmaceutical vehicles also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or pH buffering agents.

The present compositions can take the form of solutions, suspensions, emulsion, tablets, pills, pellets, capsules, capsules containing liquids, powders, sustained-release formulations, suppositories, emulsions, aerosols, sprays, suspensions, or any other form suitable for use. In one embodiment, the pharmaceutically acceptable vehicle is a capsule (see e.g., U.S. Pat. No. 5,698,155). Other examples of suitable pharmaceutical vehicles are described in “Remington's Pharmaceutical Sciences” by E. W. Martin.

In a preferred embodiment, the compounds and compositions of the invention are formulated in accordance with routine procedures as a pharmaceutical composition adapted for intravenous administration to human beings. Typically, compounds and compositions of the invention for intravenous administration are solutions in sterile isotonic aqueous buffer. Where necessary, the compositions may also include a solubilizing agent. Compositions for intravenous administration may optionally include a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the compound of the invention is to be administered by intravenous infusion, it can be dispensed, for example, with an infusion bottle containing sterile pharmaceutical grade water or saline. Where the compound of the invention is administered by injection, an ampoule of sterile water for injection or saline can be provided so that the ingredients may be mixed prior to administration.

Compounds and compositions of the invention for oral delivery may be in the form of tablets, lozenges, aqueous or oily suspensions, granules, powders, emulsions, capsules, syrups, or elixirs. Compounds and compositions of the invention for oral delivery can also be formulated in foods and food mixes. Orally administered compositions may contain one or more optionally agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, where in tablet or pill form, the compositions may be coated to delay disintegration and absorption in the gastrointestinal tract thereby providing a sustained action over an extended period of time. Selectively permeable membranes surrounding an osmotically active driving compound are also suitable for orally administered compounds and compositions of the invention. In these later platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time delay material such as glycerol monostearate or glycerol stearate may also be used. Oral compositions can include standard vehicles such as mannitol, lactose, starch, magnesium stearate, sodium saccharine, cellulose, magnesium carbonate, etc. Such vehicles are preferably of pharmaceutical grade.

The amount of a compound of the invention that will be effective in the treatment or prevention of a particular disorder or condition disclosed herein will depend on the nature of the disorder or condition, and can be determined by standard clinical techniques. In addition, in vitro or in vivo assays may optionally be employed to help identify optimal dosage ranges. The precise dose to be employed in the compositions will also depend on the route of administration, and the seriousness of the disease or disorder, and should be decided according to the judgment of the practitioner and each patient's circumstances. However, suitable dosage ranges for oral administration are generally about 0.001 milligram to 200 milligrams of a compound of the invention per kilogram body weight. In specific preferred embodiments of the invention, the oral dose is 0.01 milligram to 70 milligrams per kilogram body weight, more preferably 0.1 milligram to 50 milligrams per kilogram body weight, more preferably 0.5 milligram to 20 milligrams per kilogram body weight, and yet more preferably 1 milligram to 10 milligrams per kilogram body weight. In a most preferred embodiment, the oral dose is 5 milligrams of a compound of the invention per kilogram body weight. The dosage amounts described herein refer to total amounts administered; that is, if more than one compound of the invention is administered, the preferred dosages correspond to the total amount of the compounds of the invention administered. Oral compositions preferably contain 10% to 95% active ingredient by weight.

Suitable dosage ranges for intravenous (i.v.) administration are 0.01 milligram to 100 milligrams per kilogram body weight, 0.1 milligram to 35 milligrams per kilogram body weight, and 1 milligram to 10 milligrams per kilogram body weight. Suitable dosage ranges for intranasal administration are generally about 0.01 pg/kg body weight to 1 mg/kg body weight. Suppositories generally contain 0.01 milligram to 50 milligrams of a compound of the invention per kilogram body weight and comprise active ingredient in the range of 0.5% to 10% by weight. Recommended dosages for intradermal, intramuscular, intraperitoneal, subcutaneous, epidural, sublingual, intracerebral, intravaginal, transdermal administration or administration by inhalation are in the range of 0.001 milligram to 200 milligrams per kilogram of body weight. Suitable doses of the compounds of the invention for topical administration are in the range of 0.001 milligram to 1 milligram, depending on the area to which the compound is administered. Effective doses may be extrapolated from dose-response curves derived from in vitro or animal model test systems. Such animal models and systems are well known in the art.

The invention also provides pharmaceutical packs or kits comprising one or more containers filled with one or more compounds of the invention. Optionally associated with such container(s) can be a notice in the form prescribed by a governmental agency regulating the manufacture, use or sale of pharmaceuticals or biological products, which notice reflects approval by the agency of manufacture, use or sale for human administration. In a certain embodiment, the kit contains more than one compound of the invention. In another embodiment, the kit comprises a compound of the invention and another lipid-mediating compound, including but not limited to a statin, a thiazolidinedione, or a fibrate.

The compounds of the invention are preferably assayed in vitro and in vivo, for the desired therapeutic or prophylactic activity, prior to use in humans. For example, in vitro assays can be used to determine whether administration of a specific compound of the invention or a combination of compounds of the invention is preferred for lowering fatty acid synthesis. The compounds and compositions of the invention may also be demonstrated to be effective and safe using animal model systems.

Other methods will be known to the skilled artisan and are within the scope of the invention. 5.9. Combination Therapy

In certain embodiments of the present invention, the compounds and compositions of the invention can be used in combination therapy with at least one other therapeutic agent. The compound of the invention and the therapeutic agent can act additively or, more preferably, synergistically. In a preferred embodiment, a compound or a composition comprising a compound of the invention is administered concurrently with the administration of another therapeutic agent, which can be part of the same composition as the compound of the invention or a different composition. In another embodiment, a compound or a composition comprising a compound of the invention is administered prior or subsequent to administration of another therapeutic agent. As many of the disorders for which the compounds and compositions of the invention are useful in treating are chronic disorders, in one embodiment combination therapy involves alternating between administering a compound or a composition comprising a compound of the invention and a composition comprising another therapeutic agent, e.g., to minimize the toxicity associated with a particular drug. The duration of administration of each drug or therapeutic agent can be, e.g., one month, three months, six months, or a year. In certain embodiments, when a composition of the invention is administered concurrently with another therapeutic agent that potentially produces adverse side effects including but not limited to toxicity, the therapeutic agent can advantageously be administered at a dose that falls below the threshold at which the adverse side is elicited.

The present compositions can be administered together with a statin. Statins for use in combination with the compounds and compositions of the invention include but are not limited to atorvastatin, pravastatin, fluvastatin, lovastatin, simvastatin, and cerivastatin.

The present compositions can also be administered together with a PPAR agonist, for example a thiazolidinedione or a fibrate. Thiazolidinediones for use in combination with the compounds and compositions of the invention include but are not limited to 5-((4-(2-(methyl-2-pyridinylamino)ethoxy)phenyl)methyl)-2,4-thiazolidinedione, troglitazone, pioglitazone, ciglitazone, WAY-120,744, englitazone, AD 5075, darglitazone, and rosiglitazone. Fibrates for use in combination with the compounds and compositions of the invention include but are not limited to gemfibrozil, fenofibrate, clofibrate, or ciprofibrate. As mentioned previously, a therapeutically effective amount of a fibrate or thiazolidinedione often has toxic side effects. Accordingly, in a preferred embodiment of the present invention, when a composition of the invention is administered in combination with a PPAR agonist, the dosage of the PPAR agonist is below that which is accompanied by toxic side effects.

The present compositions can also be administered together with a bile-acid-binding resin. Bile-acid-binding resins for use in combination with the compounds and compositions of the invention include but are not limited to cholestyramine and colestipol hydrochloride. The present compositions can also be administered together with niacin or nicotinic acid. The present compositions can also be administered together with a RXR agonist. RXR agonists for use in combination with the compounds of the invention include but are not limited to LG 100268, LGD 1069, 9-cis retinoic acid, 2-(1-(3,5,5,8,8-pentamethyl-5,6,7,8-tetrahydro-2-naphthyl)-cyclopropyl)-pyridine-5-carboxylic acid, or 4-((3,5,5,8,8-pentamethyl-5,6,7,8-tetrahydro-2-naphthyl)₂-carbonyl)-benzoic acid. The present compositions can also be administered together with an anti-obesity drug. Anti-obesity drugs for use in combination with the compounds of the invention include but are not limited to β-adrenergic receptor agonists, preferably β-3 receptor agonists, fenfluramine, dexfenfluramine, sibutramine, bupropion, fluoxetine, and phentermine. The present compositions can also be administered together with a hormone. Hormones for use in combination with the compounds of the invention include but are not limited to thyroid hormone, estrogen and insulin. Preferred insulins include but are not limited to injectable insulin, transdermal insulin, inhaled insulin, or any combination thereof. As an alternative to insulin, an insulin derivative, secretagogue, sensitizer or mimetic may be used. Insulin secretagogues for use in combination with the compounds of the invention include but are not limited to forskolin, dibutryl cAMP or isobutylmethylxanthine (IBMX).

The present compositions can also be administered together with a tyrophostine or an analog thereof. Tyrophostines for use in combination with the compounds of the invention include but are not limited to tryophostine 51.

The present compositions can also be administered together with sulfonylurea-based drugs. Sulfonylurea-based drugs for use in combination with the compounds of the invention include, but are not limited to, glisoxepid, glyburide, acetohexamide, chlorpropamide, glibomuride, tolbutamide, tolazamide, glipizide, gliclazide, gliquidone, glyhexamide, phenbutamide, and tolcyclamide. The present compositions can also be administered together with a biguanide. Biguanides for use in combination with the compounds of the invention include but are not limited to metformin, phenformin and buformin.

The present compositions can also be administered together with an α-glucosidase inhibitor. α-glucosidase inhibitors for use in combination with the compounds of the invention include but are not limited to acarbose and miglitol.

The present compositions can also be administered together with an apo A-I agonist. In one embodiment, the apo A-I agonist is the Milano form of apo A-I (apo A-IM). In a preferred mode of the embodiment, the apo A-IM for administration in conjunction with the compounds of the invention is produced by the method of U.S. Pat. No. 5,721,114 to Abrahamsen. In a more preferred embodiment, the apo A-I agonist is a peptide agonist. In a preferred mode of the embodiment, the apo A-I peptide agonist for administration in conjunction with the compounds of the invention is a peptide of U.S. Pat. No. 6,004,925 or 6,037,323 to Dasseux.

The present compositions can also be administered together with apolipoprotein E (apo E). In a preferred mode of the embodiment, the apoE for administration in conjunction with the compounds of the invention is produced by the method of U.S. Pat. No. 5,834,596 to Ageland.

In yet other embodiments, the present compositions can be administered together with an HDL-raising drug; an HDL enhancer; or a regulator of the apolipoprotein A-I, apolipoprotein A-IV and/or apolipoprotein genes.

### 5.10. Combination Therapy with Cardiovascular Drugs

The present compositions can be administered together with a known cardiovascular drug. Cardiovascular drugs for use in combination with the compounds of the invention to prevent or treat cardiovascular diseases include but are not limited to peripheral antiadrenergic drugs, centrally acting antihypertensive drugs (e.g., methyldopa, methyldopa HCl), antihypertensive direct vasodilators (e.g., diazoxide, hydralazine HCl), drugs affecting renin-angiotensin system, peripheral vasodilators, phentolamine, antianginal drugs, cardiac glycosides, inodilators (e.g., amrinone, milrinone, enoximone, fenoximone, imazodan, sulmazole), antidysrhythmic drugs, calcium entry blockers, ranitine, bosentan, and rezulin.

### 5.11. Combination Therapy for Cancer Treatment

The present compositions can be administered together with treatment with irradiation or one or more chemotherapeutic agents. For irridiation treatment, the irradiation can be gamma rays or X-rays. For a general overview of radiation therapy, see Hellman, Chapter 12: Principles of Radiation Therapy Cancer, in: Principles and Practice of Oncology, DeVita et al., eds., 2^{nd}. Ed., J. B. Lippencott Company, Philadelphia. Useful chemotherapeutic agents include methotrexate, taxol, mercaptopurine, thioguanine, hydroxyurea, cytarabine, cyclophosphamide, ifosfamide, nitrosoureas, cisplatin, carboplatin, mitomycin, dacarbazine, procarbizine, etoposides, campathecins, bleomycin, doxorubicin, idarubicin, daunorubicin, dactinomycin, plicamycin, mitoxantrone, asparaginase, vinblastine, vincristine, vinorelbine, paclitaxel, and docetaxel. In a specific embodiment, a composition of the invention further comprises one or more chemotherapeutic agents and/or is administered concurrently with radiation therapy. In another specific embodiment, chemotherapy or radiation therapy is administered prior or subsequent to administration of a present composition, preferably at least an hour, five hours, 12 hours, a day, a week, a month, more preferably several months (e.g., up to three months), subsequent to administration of a composition of the invention.

### 5.12. Docking Procedures for the Identification of Non-Substrate Inhibitors of Acyl Coenzyme A Ligases and Acyl Coenzyme A Metabolizing Enzymes

The present invention is directed, in part, toward obtaining compounds useful for the prevention and treatment the conditions disclosed above. More specifically, the present invention is directed toward obtaining acyl coenzyme A mimics that are selective, non-substrate inhibitors of acyl coenzyme A ligases and acyl coenzyme A metabolizing enzymes. Identification of such inhibitors is carried out using computer-assisted methods including, but not limited to, docking procedures and the development and use of pharmacophore models.

In certain embodiments, the acyl coenzyme A metabolizing or binding proteins are acyl coenzyme A or fatty acid ligases. Exemplary acyl CoA ligases include, but are not limited to acetate-coA ligase (EC 6.2.1.1), butyrate-coA ligase (EC 6.2.1.2), long-chain-fatty-acid-coA ligase (EC 6.2.1.3), succinate-coA ligase (GDP-forming) (EC 6.2.1.4), succinate-coA ligase (ADP-forming) (EC 6.2.1.5), glutarate-coA ligase (EC 6.2.1.6), cholate-coA ligase (EC 6.2.1.7), oxalate-coA ligase (EC 6.2.1.8), malate-coA ligase (EC 6.2.1.9), acid-coA ligase (GDP-forming) (EC 6.2.1.10), biotin-coA ligase (EC 6.2.1.11), 4-coumarate-coA ligase (EC 6.2.1.12), acetate-coA ligase (ADP-forming) (EC 6.2.1.13), 6-carboxyhexanoate-coA ligase (EC 6.2.1.14), arachidonate-coA ligase (EC 6.2.1.15), acetoacetate-coA ligase (EC 6.2.1.16), propionate-coA ligase (EC 6.2.1.17), citrate-coA ligase (EC 6.2.1.18), long-chain-fatty-acid-luciferin-component ligase (EC 6.2.1.19), long-chain-fatty-acid-acyl-carrier protein ligase (EC 6.2.1.20), [citrate (pro-3S)-lyase] ligase (EC 6.2.1.22), dicarboxylate-coA ligase (EC 6.2.1.23), phytanate-coA ligase (EC 6.2.1.24), benzoate-coA ligase (EC 6.2.1.25), O-succinylbenzoate-coA ligase (EC 6.2.1.26), 4-hydroxybenzoate-coA ligase (EC 6.2.1.27), 3-alpha,7-alpha-dihydroxy-5-beta-cholestanate-coA ligase (EC 6.2.1.28), 3-alpha,7-alpha,12-alpha-trihydroxy-5-beta-cholestanate-coA ligase (EC 6.2.1.29), phenylacetate-coA ligase (EC 6.2.1.30), 2-furoate-coA ligase (EC 6.2.1.31), anthranilate-coA ligase (EC 6.2.1.32), 4-chlorobenzoate-coA ligase (EC 6.2.1.33), and trans-feruloyl-coA synthase (EC 6.2.1.34). Methods of isolation and/or determining binding to and/or measuring activity of an acyl coenzyme A ligase are described in Aas and Bremer, 1968, Biochim Biophys Acta 164(2):157-66; Barth et al., 1971, Biochim Biophys Acta 248(1):24-33; Groot, 1975, Biochim Biophys Acta 380(1):12-20; Scholte et al., 1971, Biochim Biophys Acta 231(3):479-86; Scholte and Groot, 1975, Biochim Biophys Acta 409(3):283-96; Scaife and Tichivangana, 1980, Biochim Biophys Acta. 619(2):445-50; Man and Brosnan, 1984, Int J Biochem. 1984;16(12):1341-3; Patel and Walt, 1987, J Biol Chem. 262(15):7132-4; Philipp and Parsons, 1979, J Biol Chem. 254(21):19785-90; Vanden Heuvel et al., 1991, Biochem Pharmacol. 42(2):295-302; Youssef et al., 1994, Toxicol Lett. 74(1):15-21; and Vessey et al., 1999, Biochim Biophys Acta1428(2-3):455-62.

In other embodiments, the acyl coenzyme A metabolizing or binding proteins are enzymes or proteins involved in reactions utilizing acyl carrier protein (ACP). Exemplary ACPs include, but are not limited to, [acyl-carrier-protein] acetyltransferase (EC 2.3.1.38), [acyl-carrier-protein] malonyltransferase (EC 2.3.1.39), [acyl-carrier-protein] phosphodiesterase (EC 3.1.4.14); enoyl-[acyl-carrier-protein] reductase (NADPH) (EC 1.3.1.10), holo-[acyl-carrier-protein] synthase (EC 2.7.8.7), 3-oxoacyl-enzyme [acyl-carrier protein], 3-oxoacyl-[acyl-carrier-protein] reductase (EC 1.1.1.100), or 3-oxoacyl-[acyl-carrier-protein] synthase (EC 2.3.1.41).

In yet other embodiments, the acyl coenzyme A metabolizing or binding proteins are enzymes or proteins involved in reactions using Coenzyme A. Exemplary enzymes or proteins involved in reactions using Coenzyme A include, but are not limited to, acetate-coA ligase (EC 6.2.1.1), acetoacetyl-coA hydrolase (EC 3.1.2.11), acetoacetyl-coA: acetate coA transferase (EC 2.8.3.8), acetyl-coA acetyltransferase [thiolase] (EC 2.3.1.9), acetyl-coA acyltransferase (EC 2.3.1.16), acetyl-coA carboxylase (EC 6.4.1.2), [acetyl-coA carboxylase] phosphatase (EC 3.1.3.4), acetyl-coA ligase (EC 6.2.1.1), acyl-coA acyltransferase (EC 2.3.1.16), acyl-coA dehydrogenase (EC 1.3.99.3), acyl-coA dehydrogenase (NADP+) (EC 1.3.1.8), butyryl-coA dehydrogenase (EC 1.3.99.2), cholate-coA ligase (EC 6.2.1.7), dephospho-coA kinase (EC 2.7.1.24), enoyl-coA hydratase (EC 4.2.1.17), formyl-coA hydrolase (EC 3.1.2.10), glucan-1,4-a-glucosidase [glucoAmylase] (EC 3.2.1.3), glutaryl-coA dehydrogenase (EC 1.3.99.7), glutaryl-coA ligase (EC 6.2.1.6), 3-hydroxyacyl-coA dehydrogenase (EC 1.1.1.35), 3-hydroxybutyryl-coA dehydratase (EC 4.2.1.55), 3-hydroxybutyryl-coA dehydrogenase (EC 1.1.1.157), 3-hydroxyisobutyryl-coA hydrolase (EC 3.1.2.4), hydroxymethylglutaryl-coA lyase (EC 4.1.3.4), hydroxymethylglutaryl-coA reductase (EC 1.1.1.88), hydroxymethylglutaryl-coA reductase (NADPH) (EC 1.1.1.34), [hydroxymethylglutaryl-coA reductase (NADPH)] kinase (EC 2.7.1.109), [hydroxymethylglutaryl-coA reductase (nadph)] phosphatase (EC 3.1.3.47), hydroxymethylglutaryl-coA synthase (EC 4.1.3.5), lactoyl-coA dehydratase (EC 4.2.1.54), malonate-coA transferase (EC 2.8.3.3), malonyl-coA decarboxylase (EC 4.1.1.9), methylcrotonyl-coA carboxylase (EC 6.4.1.4), methylglutaconyl-coA hydratase (EC 4.2.1.18), methylmalonyl-coA carboxyltransferase (EC 2.1.3.1), methylmalonyl-coA decarboxylase (EC 4.1.1.41), methylmalonyl-coA epimerase (EC 5.1.99.1), methylmalonyl-coA mutase (EC 5.4.99.2), oxalate-coA transferase (EC 2.8.3.2), oxalyl-coA decarboxylase (EC 4.1.1.8), 3-oxoacid-coA transferase (EC 2.8.3.5), 3-oxoadipate coA-transferase (EC 2.8.3.6), palmitoyl-coA-enzyme palmitoyltransferase, propionate-coA ligase (EC 6.2.1.17), propionyl-coA carboxylase (EC 6.4.1.3), succinate-coA ligase (ADP-forming) (EC 6.2.1.5), succinate-coA ligase (GDP-forming) (EC 6.2.1.4), or succinate-propionate coA transferase.

In yet other embodiments, the acyl coenzyme A metabolizing or binding proteins are enzymes or proteins involved in reactions resulting in the biosynthesis or degradation of coA. Exemplary enzymes or proteins involved in reactions resulting in the biosynthesis or degradation of coA include, but are not limited to, pantothenatekinase (EC 2.7.1.33), pantothenate-B-alanine ligase (EC 6.3.2.1), phosphopantothenate-cysteine ligase (EC 6.3.2.5), pantetheine kinase (EC 2.7.1.34), pantetheine-phosphate adenylyltransferase (EC 2.7.7.3), 2-dehydropantoate reductase (EC 1.1.1.169), pantothenase (EC 3.5.1.22), pantothenoylcysteine decarboxylase (EC 4.1.1.30), phosphopantothenate-cysteine ligase (EC 6.3.2.5), phosphopantothenoylcysteine decarboxylase (EC 4.1.1.36).

In yet other embodiments, the acyl coenzyme A metabolizing or binding proteins are enzymes or proteins involved in the “mevalonate shunt,” as described in Edmond and Popjak, 1974, J. Biol. Chem. 249:66-71

In specific embodiments, the present invention is directed toward obtaining acyl coenzyme A mimics that are selective, non-substrate inhibitors of short-chain acyl coenzyme A ligases and of short-chain acyl coenzyme A metabolizing enzymes.

Docking procedures involve inter alia the computer-assisted determination and evaluation of the interaction between a biological macromolecule and a ligand. In certain embodiments, the biological macromolecule is an enzyme and the ligand may be a substrate, or a non-substrate inhibitor, of that enzyme. Non-substrate inhibitors can be, but are not limited to, structural analogs or molecular mimics, in whole or in part, of a natural substrate of the enzyme. Accordingly, docking procedures are used in the present invention both qualitatively and quantitatively for the identification of putative inhibitors of, e.g., short-chain acyl coenzyme A ligases and of short-chain acyl coenzyme A metabolizing enzymes. Such docking procedures are also used to evaluate the binding of those putative identified inhibitors to long-chain acyl coenzyme A ligases and long-chain acyl coenzyme A metabolizing enzymes. Comparison of the relative binding strength of the identified, putative inhibitors to each class of acyl coenzyme A binding enzyme provides an indication of the specificity and selectivity of the inhibitor.

The docking procedures of the present invention employ computation tools for the identification and evaluation of energetically favorable binding interactions between a biological macromolecule and a ligand that have been shown to be useful for structure-based drug design, such as those disclosed in U.S. Pat. Nos. 5,866,343, 6,341,256 B1, and 6,365,626 B1, each of which is hereby incorporated by reference in its entirety. The docking approaches useful in different aspects of the present invention fall into two main categories, namely, qualitative and quantitative methods. Qualitative methods are restricted primarily to calculations based on shape, complementarity and consist of finding the best fit between two shapes, which can be carried out, in one non-limiting approach, using the computer program called “Dock,” as described B. K. Shoichet et al. (Shichet et al., Protein Engineering, 7: 723-732, 1993, which is hereby incorporated by reference in its entirety). Quantitative methods useful in the docking methods of the present invention are based primarily on energy calculations designed to determine the global minimum energy of the ligand binding interaction with the protein target. One non-limiting description of a method useful in this aspect of the invention is provided by Kollman (Kollam, Chem. Rev. 93: 2395-2417, 1993, which is hereby incorporated by reference in its entirety). Moreover, the docking methods of the present invention further comprise hybrid methods in which an interaction energy is calculated for the binding of a target protein and an individual fragment of a putative ligand; the resulting data are then assembled based on shape, complementarity criteria to form new ligand molecules. This aspect of the present invention uses, in one non-limiting example, the approach described by P. A. Goodford (Goodford, J. Med. Chem, 28: 849-857, 1985, which is hereby incorporated by reference in its entirety).

By using the docking methods of the present invention, intermolecular movement between the biological macromolecule and ligand are simulated by computing intermolecular forces to evaluate preferred “docking” interactions between the molecules. According to these methods, the energy of the interaction between the two molecules is calculated in order to define, as the best binding site interactions, those which have the most favorable or minimum potential energy. That is, it is possible to rank a series of putative ligands with respect to their relative ability to bind to the biological macromolecule. Moreover, therefore, it is also possible to compare the strength of the interaction of a given ligand with two different biological macromolecules, e.g., a short-chain acyl coenzyme A ligase and a long-chain acyl coenzyme A ligase. It should be noted that the predictive accuracy of any such quantitative method is limited by the resolution or precision of the model. In most calculations of such binding interactions, the molecular structures are mapped onto a grid. This mapping is performed either with or without a transfer function, e.g. a 1/r-function in the case of electrostatic potential description. The calculation of the interaction between the two biological macromolecule and the ligand, such as calculating the potential energy between the two molecules, is performed for each relative position of the two molecules, namely, each relative translational position and each rotational orientation between the two molecules.

In a preferred embodiment, therefore, the docking methods of the present invention make use of correlation between a potential grid, which represents one molecule, and an interaction field grid, which represents the second molecule, to obtain for each selected relative rotation between the two molecules, a potential energy that represents a binding energy of the two molecules for relative translational positions in space between the two molecules. Therefore, by using a single complex correlation calculation for each relative rotation between the two molecules, the resulting grids can be scanned to obtain the most energetically favorable binding interaction between two molecules. More specifically, by using a grid resolution in the range of 0.25 Å-0.45 Å, this approach provides very acceptable quantitative results for determining molecule binding energy for all relative translational positions in space between the two molecules.

Therefore, in one embodiment the present invention, docking methods are employed that provide a quantitative value for an energetically favorable binding interaction between two molecules, i.e. a biological macromolecule and a ligand. In a specific embodiment of the present invention, the biological macromolecule is involved in the synthesis and or metabolism of an acyl coenzyme A compound while the ligand is an acyl coenzyme A mimic that binds to and/or inhibits the enzyme. One such method comprises the steps of: a) obtaining potential energy structural data for each atom site in the molecules; b) selecting a grid resolution corresponding to a sampling grid size substantially smaller than an average distance between bonded atoms in the molecules; c) selecting a range of relative rotations between the two molecules; d) mapping a plurality of potential energy field components of one of the molecules onto a corresponding one of a plurality of energy field component grids having the resolution with one molecule at a predetermined rotation and position, wherein each grid point of the component grids has a potential energy value interpolated from the potential energy structural data; e) mapping a plurality of interaction field components of another of the molecules onto a corresponding one of a plurality of interaction component grids having the resolution with the other molecule at a predetermined rotation and position, the interaction component corresponding to coefficients of a forcefield between the molecules, wherein each grid point of the component grids has an interaction value interpolated from the potential energy structural data; f) calculating a correlation between each potential energy field component grid and each interaction field component grid to obtain a grid of molecule binding energy values representing a binding energy of the two molecules in the relative rotation for relative translational positions in space between the molecules; g) determining at least one maximum of the binding energy values and recording the relative translational positions for the maximum binding energy values; h) rotating at least one of the molecules according to each relative rotation in the range, repeating the step of mapping for the at least one of the molecules and subsequently repeating the steps (f) and (g) of calculating and determining for each relative rotation; and i) selecting an energetically favorable one of the relative rotations in the range and the relative translational positions based on the maximum binding energy values to generate the position value for an energetically favorable binding site between the two molecules.

Therefore, according this method, only one molecule, e.g., the biological macromolecule, needs to be rotated relative to the other, e.g. the ligand which is a putative inhibitor of the biochemical activity of the biological macromolecule. Consequently, the map of one of the molecules can be used repeatedly while the map of the second molecule can be recalculated for each new rotational position. That is, the map of the target macromolecule can be used repeatedly, while that for each ligand/putative inhibitor is varied. Since the interaction field components are easier to map, it is preferred that only the interaction component grids be remapped for each new rotation. Also preferably, the preferred transform for carrying out the correlation is the discrete Fourier transform.

Preferably, the potential energy field components consist of the electrostatic potential which is based on Coulomb's law and varies as a function of 1/r, a second component for the first Van der Waals term A, which varies as a function of 1/r¹² and a third component for the second Van der Waals term B, which varies as a function of 1/r⁶. The result of the correlation for each field component must be summed with the results of the other components in order to obtain a total binding energy of the two molecules for the given relative rotation and for each relative translational position in space provided within the grid.

The docking methods of the present invention are directed toward obtaining and evaluating interactions between ligands, which may be non-substrate inhibitors, and biological macromolecules which are proteins, and more specifically, are short-chain acyl coenzyme A ligases, long-chain acyl coenzyme A ligases, short-chain acyl coenzyme A metabolizing enzymes, and long-chain acyl coenzyme A metabolizing enzymes. The potential energy of the system consisting of the protein and ligand is calculated by determining the potential energy field created by the protein and then calculating the potential energy resulting from the contribution of each atom in the ligand for a particular position in space within the potential energy field of the protein. The potential energy is calculated using three basic terms. The first term is the electrostatic potential. This results from an electrostatic charge at a particular atom within the ligand interacting with the electrostatic field potential created by the molecule. Such potentials are greater in polar or ionic molecules. The second and third potential energy terms come from the Van der Waals potentials, which is generally the 6-12 Lennard Jones potential. The combination of the three potential energy terms are used to provide a potential energy minimum (maximum binding energy) as a particular radial distance. Potential terms can be extended by an explicit term for hydrogen bond interaction, using, as one non-limiting example, the methods and approaches disclosed in U.S. Pat. Nos. 5,642,292, and 6,308,145 B1, each of which is hereby incorporated by reference in its entirety.

For the chosen protein and the chosen ligand/putative inhibitor, data concerning static charge at the atom sites in the molecules as well as the coefficients for the Van der Waals forces are obtained from existing databases. Such potential energy structural data is originally determined empirically and/or by theoretical model calculations. Next, a grid resolution corresponding to a sampling grid size substantially smaller than an average distance between atoms in the molecules is selected. A sampling grid size of 0.4 Å provides, in most cases, sufficiently high resolution to obtain good results for protein ligand pairs. A grid resolution of 0.25 Å, while computationally more intensive, provides substantially more accurate results.

Once the grid resolution is selected, each potential energy field component of one of the molecules, in the preferred embodiment the protein, is mapped onto a corresponding energy field component grid. This typically involves calculating for each grid point the potential energy field created by each atom site in the protein and summing all potentials to obtain the field potential. Since this step of mapping may only be carried out once for each target protein, the effect of every atom site in the protein may be taken into account and all of the computation time required may be taken. For atoms very close to a grid point, where computational errors can result from selection outside the representation range of numbers in a computer, an arbitrary high value for their contribution to the potential field is taken. The relative spatial coordinates of each atom site for the protein and for the ligand are known from the structural data obtained from existing databases, or from predicted structural data.

The ligands, which can be non-substrate inhibitors of the enzymes indicated above, are generally much smaller molecule and therefore are easier to map onto the grid. The potential energy field components are not mapped onto the grid but rather the interaction field components are mapped onto the grid. The interaction field components relate to the charge quantities in the case of the electrostatic potential and the Van der Waals coefficients in the case of the Van der Waals potentials. For each atom site, the coefficients associated therewith are mapped onto the grid points surrounding each atom site in virtual space. The interpolation method for such mapping may be trilinear or a Gaussian distribution. Calculation of the values for the interaction field grid relating to the ligand involves carrying out a series of simple calculations with respect to each atom site in the ligand. The interaction component grids are built up for the particular rotational orientation of the ligand within the grid space by calculating the interaction field components for all of the atom sites in the ligand.

Since the potential energy field grids and the corresponding interaction field component grids have the same grid resolution and grid size, a correlation between the two grids may be calculated. In a preferred embodiment, the discrete Fourier transform using a fast Fourier transform method is applied to each grid. The two transformed grids are then multiplied using element by element multiplication to obtain an intermediate product grid, and then the intermediate product grid is subjected to an inverse fast Fourier transform to obtain a grid representing for each point in the grid a binding energy for each component for each translational position in space between the protein and the ligand. By summing the resulting component grids for the binding energies, a single total binding energy grid is obtained. The total binding energy grid is scanned to determine a maximum binding energy value for the particular rotation of the ligand. As can also be appreciated, if an atom site happens to fall directly on a grid point as a result of the virtual rotation, the computational accuracy is not compromised. For this reason, it is further preferred to rotate the molecule whose interaction field components are being calculated and mapped onto the grid rather than rotating the molecule whose potential energy field components are being mapped. The method described thus far is carried out for every conceivable relative rotation between the protein and the ligand. Since, in many cases, the ligands/putative inhibitors of the present invention are structural analogs or molecular mimics, in whole or in part, of coenzyme, A, and the interaction between the enzyme and coenzyme A may have been previously characterized, not all possible orientations need be examined.

Since, generally, only a small part of the protein will adjust to a different conformation on the incoming ligand, potential energy components are then preferably mapped in two parts. First the potential energy field grid is mapped for the larger part of the protein which does not change conformation, and this first grid is stored and reused each time. To calculate the total potential energy field grid for each conformation of the protein, the potential energy grid for the second part of the protein, which has assumed a different conformation, is calculated. The potential energy field grid of the first part is added to the potential energy grid of the second part to obtain the total potential energy field grid for the protein in the conformational state. This method of mapping the potential energy component grids is preferred because the computational time required to map the potential energy components onto the component grids is significant for larger molecules.

In one embodiment of, the docking methods of the present invention are applied using, as the biological macromolecular component of the interaction, a short-chain acyl coenzyme A ligase, such as but not limited to a short chain acyl coenzyme A synthetase or butyrate-CoA ligase. In another embodiment, the biological macromolecular component of the interaction, is a short-chain acyl coenzyme A metabolizing enzyme selected from the group consisting of aceto acetyl-CoA thiolase, HMG-CoA synthase, and HMG-CoA reductase. In each of these embodiments, putative inhibitors, which are ligands identified by virtue of the computed binding energy of their interaction with the biological macromolecule examined, are docked, using the same methods to one or more long-chain acyl coenzyme A ligases and/or one or more long-chain acyl coenzyme A metabolizing enzymes, such as, but not limited to those selected from the group consisting of fatty acyl CoA synthetase and palymitoyl CoA synthetase long chain acyl-CoA oxidase, long-chain enoyl-CoA hydratase, and long chain hydoxyacyl CoA dehydrogenase.

In another embodiment of the present invention, which is particularly useful for screening purposes for obtaining non-substrate inhibitors useful for treatment of the conditions disclosed above, a consensus three-dimensional structure is constructed for each of the following enzymes: (a) short-chain acyl coenzyme A ligase, (b) a short-chain acyl coenzyme A ligase, (c) a long-chain acyl coenzyme A ligase, and (d) a long-chain acyl coenzyme A metabolizing enzyme. The construction of such consensus structures is facilitated by the existence of publically-avail able crystal structures for representative enzymes. Moreover, since these structures include complexes of the enzyme and substrate, conformational alterations resulting from substrate binding, as well as the delineation of the substrate-binding site, and amino acid residues involved in and/or critical to that binding, may be inferred by those skilled in the art. See for example, the structures provided by the Protein Data Bank (http://rutgers.rcsb.org/pdb) and described by Berman et al. (Berman et al. 2000, Nucleic Acids Research 28(1): 235-42).

For example, such a consensus structure may be constructed by superimposing the coordinates each of the crystal structures that are publically available using the InsightII computer program ((1996), Molecular Simulations, Inc., San Diego, Calif.) to provide the best overall structural comparison, in which each of the input amino acid sequences are aligned based on the superimposition of their structures. Such sequence alignment accommodates such features as loops in a protein which differ from the other protein sequences. The structural superimposition is performed using the Homology module of the InsightII ((1996), Molecular Simulations, Inc., San Diego, Calif.) program and, in one non-limiting example, a Silicon Graphics INDIGO2 computer (Silicon Graphics Inc., Mountain View, Calif.). The sequence alignment can be manually adjusted and sequence variation profile can be provided for each input amino acid sequence. The sequence variation profile can then be used for comparing the consensus structure so determined with each new protein to be examined. In this procedure, the sequence of a target protein is read into the program and manually aligned with the known proteins based on the sequence variation profile described previously. A set of three-dimensional coordinates can then be assigned to a target protein using the Homology module of the InsightII program ((1996), Molecular Simulations, Inc., San Diego, Calif.). The coordinates for loop regions resulting, e.g. in a new, target protein, resulting from an insertion a number of amino acids, can be automatically generated by the computer program and manually adjusted to provide a more ideal geometry using the program CHAIN (Sack, J. S. (1988) J. Mol. Graphics 6, 244-245). Finally, the molecular model derived for the new target protein is subjected to energy minimization using the X-plor program (Brunger, A. T. (1992), New Haven, Conn.) so that any steric strain introduced during the model-building process is be relieved. Such a model can then be screened for unfavorable steric contacts and if necessary such side chains were remodeled either by using a rotamer library database or by manually rotating the respective side chains. A molecular structure constructed in this manner can then be used in the docking procedures described above to obtain the desired inhibitors.

If the three dimensional structure of a ligand is not known, one can use one or more computer programs, including but not limited to, CATALYST (Molecular Simulations, Inc., San Diego, Calif.), to predict the three-dimensional structure of the compound. Three-dimensional conformers are generated from a starting structure using software well known in the art such as, but not limited to, the Best or Fast Conformational Analyses (Molecular Simulations, Inc., San Diego, Calif.). In addition, where the ligand or putative inhibitor is a structural analog or molecular mimic of all or part of a natural substrate of the target enzyme, the three-dimensional structure of that substrate can-be used to predict the three-dimensional structure of the subject ligand. This is particularly helpful where the three-dimensional structure of the natural substrate has been established by X-ray crystallography of an enzyme-substrate complex.

In one embodiment, analysis of such is carried out using the Docking module within the program INSIGHTII and using the Affinity suite of programs for automatically docking a ligand to the biological macromolecule i.e. enzyme. As notes above, hydrogen atoms on the ligand and enzyme are generated and potentials are assigned to both enzyme and ligand prior to the start of the docking procedure. The docking method in the InsightII program uses the CVFF force field and a Monte Carlo search strategy to search for and evaluate docked structures. While the coordinates for the bulk of the receptor are kept fixed, a defined region of the substrate-binding site is allowed to relax, thereby permitting the protein to adjust to the binding of different inhibitors. A binding set is defined within a distance of 5 Å from the inhibitor, allowing residues within this distance to shift and/or rotate to energetically favorable positions to accommodate the ligand. An assembly is defined consisting of the receptor and inhibitor molecule and docking performed using the fixed docking mode. Calculations approximating hydrophobic and hydrophilic interactions are used to determine the ten best docking positions of each ligand enzyme's substrate-binding site. The various docked positions of ligand are qualitatively evaluated using Ludi (Bohm, H. J. (1992) J. Comput. Aided Mol. Des. 6(6): 593-606; and Bohm, H. J. (1994) J. Comput. Aided Mol. Des. 8(3): 243-56) in INSIGHTII which can be used to estimate a binding constant (Kᵢ) for each compound in order to rank their relative binding capabilities and predicted inhibition of the target enzyme examined. The Kᵢ trends for ligands are compared with the trend of experimentally determined ligands/inhibitors in order to elucidate the structure-activity relationships (SAR) determining the potency of the ligands/inhibitors tested.

In another aspect of the present invention, the three-dimensional structure of the target enzyme, and more particularly, the substrate-binding site of that enzyme is inferred by comparing the amino acid sequence of that target protein to a homolog for which a crystal structure has been determined. In a still further aspect of the present invention, the three-dimensional structure of the target enzyme, and more particularly, the substrate-binding site of that enzyme, is determined by determining the structure using X-crystallography, NMR, or a combination of such methods, that are well known in the art.

5.13. Pharmacophore Models and Use Thereof for the Identification of Non-Substrate Inhibitors of Short-Chain Acyl Coenzyme A Ligases and Short-Chain Acyl Coenzyme A Metabolizing Enzymes

In yet another aspect of the present invention, the structure of the target enzyme is not determined a priori. Rather, desired compounds, which are non-substrate inhibitors of short-chain acyl coenzyme A ligases and/or short-chain acyl coenzyme A metabolizing enzymes but are not inhibitors of long-chain acyl coenzyme A ligases and/or long-chain acyl coenzyme A metabolizing enzymes, are identified by constructing one or more pharmacophore models and then using those models to search databases of three-dimensional structures for compounds corresponding to the pharmaocophore. Compounds identified in this manner may then be used in the docking methods described above, or as lead compounds for the design and synthesis of inhibitors that may be tested in animal model systems, tissue extracts, or in vitro assay systems using purified enzymes, as disclosed herein. Methods useful for the construction and use of a pharmacophore model for the identification of ligands/inhibitors that bind target biological macromolecules are described in U.S. Pat. No. 6,365,626 B1, which is hereby incorporated by reference in its entirety.

Pharmacophore models are used to describe compounds on the basis of shared chemical features among identified inhibitors that are inferred to be critical to the binding interactions between the ligand/inhibitor and the chemical substructures within the substrate-binding site of the protein (e.g. see Tomioka et al., (1994) J. Comput. Aided. Mol. Des. 8(4): 347-66; Greene et al. (1994) J. Chem. Inf. Comput. Sci. 34: 1297-1308).

Accordingly, compounds useful in the methods of the present invention for the prevention and treatment of the conditions disclosed herein are identified in certain embodiments using computer-assisted methods that detect potential acyl CoA mimics that are selective inhibitors of enzymes forming and/or metabolizing short chain acyl CoA compounds. Such methods can comprise accessing a database of compounds which contains structural information for the compounds in the database and comparing the compounds in the database with a pharmacophore to obtain compounds having the features common to a collection of known acyl coenzyme A mimics that are selective inhibitors of short chain acyl coenzyme A formation and/or metabolism.

Such structural comparisons can be carried out using the software described above, generally using the default parameters supplied by the manufacturer. Such parameters, however, can be modified where desired. The number of hits to be found in a given database may be influenced by the nature of the pharmacophore or query structure used, the software employed, and the constraints applied to the searches performed by that software.

The computer-assisted methods used in combination with the pharmacophores described above provide those skilled in the art with a tool for obtaining compounds that can then be evaluated for activity, either in vivo or in vitro, using the assay systems disclosed herein. For example, those skilled in the art can use pharmacophores in conjunction with a computational computer program, such as CATALYST (Molecular Simulations, Inc., San Diego, Calif.), to search databases of existing compounds for compounds that fit a derived pharmacophore and that have the desired inhibitory activity. The degree of fit of an experimental compound structure to a pharmacophore is calculated using computer-assisted methods to determine whether the compound possesses the chemical features of the pharmacophore and whether the features can adopt the necessary three-dimensional arrangement to fit the model. The computer output provides information regarding those features of the pharmacophore that are fit by an experimental compound. A compound “fits” the pharmacophore if it has the features of the pharmacophore.

Computer programs useful for searching databases of chemical compounds useful in the methods of the present invention include ISIS (MDL Information Systems, Inc., San Leandro, Calif.), SYBYL (Tripos, Inc., St. Louis, Mo.), INSIGHT II (Pharmacopeia, Inc., Princeton, N.J.), and MOE (Chemical Computing Group, Inc., Montreal, Quebec, Canada). Examples of databases of chemical compounds that can be searched using such structure-recognition software include, but are not limited to the BioByte MasterFile (BioByte Corp., Claremont, Calif.), NCI (Laboratory of Medicinal Chemistry, National Cancer Institute, NIH, Frederick, Md.), Derwent (Derwent Information, London, UK) and Maybridge (Maybridge plc, Trevillett, Tintagel, Cornwall, UK) databases, which are available from Pharmacopeia, Inc., Princeton, N.J.). Software-assisted searches of chemical databases for compounds of the present invention can be performed using a wide variety of computer workstations or general purpose computer systems.

### 5.14. Biological Methods of Identifying Acyl Coenzyme a Mimics

The present invention provides biological assays for obtaining and identifying acyl coenzyme A mimics that are useful for treating or preventing a condition of the invention.

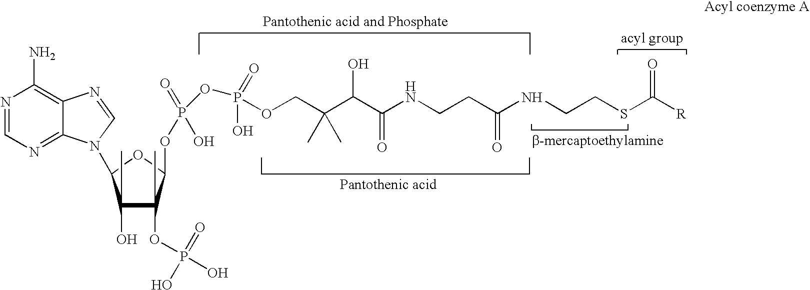

Without being bound by any theory, the present inventors believe that acyl coenzyme A mimics that bind to and/or inhibit the activity of acyl coenzyme A metabolizing or binding proteins are useful in treating or preventing diseases of the invention. As used herein the phrase “acyl coenzyme A mimic” also includes compounds that are mimics and analogs of coenzyme A as well as analogs of portions of coenzyme A, such as but not limited to the pantothenic acid portion of coenzyme A, including, but not limited to phosphorylated derivatives of pantothenic acid and analogs thereof.

Methods of measuring the binding or inhibition of acyl coenzyme A metabolizing or binding proteins by an acyl coenzyme A mimic are well known in the art. In certain embodiments, said binding or inhibition is measured by high pressure liquid chromatography, thin layer chromatography, mass spectrometry. The assays can be carried out on cellular extracts containing the acyl coenzyme A metabolizing or binding proteins or on purified, for example recombinantly expressed, acyl coenzyme A metabolizing or binding proteins.

In a preferred embodiment, the acyl coenzyme A mimic is a competitive inhibitor of acyl coenzyme A, and is most preferably a competitive inhibitor of acetyl coenzyme A. To determine whether a coenzyme A mimic is a competitive inhibitor of coenzyme A, the binding of the mimic to a fatty acid ligase is determined at two different concentrations of acyl coenzyme A. Compounds whose binding to the ligase is reduced at greater concentrations of acyl coenzyme A are competitive inhibitors of acyl coenzyme A. In other embodiments, the acyl coenzyme A mimic is a non-competitive inhibitor of acyl coenzyme A, preferably of acetyl coenzyme A. In yet other embodiments, the acyl coenzyme A mimic is an allosteric inhibitor of acyl coenzyme A, preferably of acetyl coenzyme A.

Test compounds that can be used in the present methods can include any compound from any source, including but not limited to compound libraries. The compounds can assayed singly or in multiplex format assays.

In certain embodiments, the acyl coenzyme A metabolizing or binding proteins are acyl coenzyme A or fatty acid ligases. Exemplary acyl CoA ligases include, but are not limited to acetate-CoA ligase (EC 6.2.1.1), butyrate-CoA ligase (EC 6.2.1.2), long-chain-fatty-acid-CoA ligase (EC 6.2.1.3), succinate-CoA ligase (GDP-forming) (EC 6.2.1.4), succinate-CoA ligase (ADP-forming) (EC 6.2.1.5), glutarate-CoA ligase (EC 6.2.1.6), cholate-CoA ligase (EC 6.2.1.7), oxalate-CoA ligase (EC 6.2.1.8), malate-CoA ligase (EC 6.2.1.9), acid-CoA ligase (GDP-forming) (EC 6.2.1.10), biotin-CoA ligase (EC 6.2.1.11), 4-coumarate-CoA ligase (EC 6.2.1.12), acetate-CoA ligase (ADP-forming) (EC 6.2.1.13), 6-carboxyhexanoate-CoA ligase (EC 6.2.1.14), arachidonate-CoA ligase (EC 6.2.1.15), acetoacetate-CoA ligase (EC 6.2.1.16), propionate-CoA ligase (EC 6.2.1.17), citrate-CoA ligase (EC 6.2.1.18), long-chain-fatty-acid-luciferin-component ligase (EC 6.2.1.19), long-chain-fatty-acid-acyl-carrier protein ligase (EC 6.2.1.20), [citrate (pro-3S)-lyase] ligase (EC 6.2.1.22), dicarboxylate-CoA ligase (EC 6.2.1.23), phytanate-CoA ligase (EC 6.2.1.24), benzoate-CoA ligase (EC 6.2.1.25), O-succinylbenzoate-CoA ligase (EC 6.2.1.26), 4-hydroxybenzoate-CoA ligase (EC 6.2.1.27), 3-alpha,7-alpha-dihydroxy-5-beta-cholestanate-CoA ligase (EC 6.2.1.28), 3-alpha,7-alpha,12-alpha-trihydroxy-5-beta-cholestanate-CoA ligase (EC 6.2.1.29), phenylacetate—CoA ligase (EC 6.2.1.30), 2-furoate-CoA ligase (EC 6.2.1.31), anthranilate-CoA ligase (EC 6.2.1.32), 4-chlorobenzoate-CoA ligase (EC 6.2.1.33), and trans-feruloyl-CoA synthase (EC 6.2.1.34). Methods of isolation and/or determining binding to and/or measuring activity of an acyl coenzyme A ligase are described in Aas and Bremer, 1968, Biochim Biophys Acta 164(2):157-66; Barth et al., 1971, Biochim Biophys Acta 248(1):24-33; Groot, 1975, Biochim Biophys Acta 380(1):12-20; Scholte et al., 1971, Biochim Biophys Acta 231(3):479-86; Scholte and Groot, 1975, Biochim Biophys Acta 409(3):283-96; Scaife and Tichivangana, 1980, Biochim Biophys Acta. 619(2):445-50; Man and Brosnan, 1984, Int J Biochem. 1984;16(12):1341-3; Patel and Walt, 1987, J Biol Chem. 262(15):7132-4; Philipp and Parsons, 1979, J Biol Chem. 254(21):19785-90; Vanden Heuvel et al., 1991, Biochem Pharmacol. 42(2):295-302; Youssef et al., 1994, Toxicol Lett. 74(1):15-21; and Vessey et al., 1999, Biochim Biophys Acta 1428(2-3):455-62. In certain specific embodiments, the fatty acid ligases are short chain fatty acid ligases. In such embodiments, preferred acyl coenzyme A mimics preferentially bind to or inhibit the activity of a short chain fatty acid ligase relative to a long chain fatty acid ligase.

Preferential binding by the acyl coenzyme A mimic to a short chain fatty acid ligase relative to a long chain fatty acid ligase means that the acyl coenzyme A mimic binds to the short chain fatty acid ligase with at least a 3-fold greater affinity more preferably with at least a 5-fold greater affinity, and most preferably with at least a 10-fold greater affinity than to the long chain fatty acid ligase. Preferential inhibition of a short chain fatty acid ligase relative to a long chain fatty acid ligase by the acyl coenzyme A mimic means that a particular amount or concentration of the acyl coenzyme A mimic inhibits the activity of the short chain fatty acid ligase by a degree of at least 50% more, more preferably at least 70% more, and yet more preferably at least 90% more than it inhibits the activity of the long chain fatty acid ligase. Thus, if an acyl coenzyme A mimic inhibits the activity of a a long chain fatty acid ligase by 40% at a given concentration, then the acyl coenzyme A mimic is said to inhibit the activity of the short chain fatty acid ligase by a degree of at least 50% more than it inhibits the activity of the long chain fatty acid ligase if it does so by 60% (40% +(50% x 40%)).

As used herein, a short chain fatty acid ligase is an enzyme that catalyzes the addition of coenzyme A to an acyl coenzyme A molecule in which the acyl group comprises less than eight to ten carbon atoms. Further, as used herein, a long chain fatty acid ligase is an enzyme that catalyzes the addition of coenzyme A to an acyl coenzyme A molecule in which the acyl group comprises greater than twelve to sixteen carbon atoms.

In one embodiment, a biological sample known or suspected to have fatty acid ligase activity, most preferably short chain and long chain fatty acid ligase activity, is contacted with the test compound and the output of the ligase activity (i.e., measurement of acyl coenzyme A synthesis) or binding to the ligase by the test compound is measured. In one embodiment, the biological sample is a liver extract, for example a beef liver extract (see Mahler et al., 1953, J. Biol. Chem. 204:453-468), or an adipose tissue extract. In another embodiment, the biological sample is a mitochondrial extract, a cytosol extract, a smooth endoplasmic reticulum extract, a microsomal extract, or a peroxisomal extract.

In other embodiments, the acyl coenzyme A metabolizing or binding proteins are enzymes or proteins involved in reactions utilizing acyl carrier protein (ACP). Exemplary ACPs include, but are not limited to, [acyl-carrier-protein] acetyltransferase (EC 2.3.1.38), [acyl-carrier-protein] malonyltransferase (EC 2.3.1.39), [acyl-carrier-protein] phosphodiesterase (EC 3.1.4.14); enoyl-[acyl-carrier-protein] reductase (NADPH) (EC 1.3.1.10), holo-[acyl-carrier-protein] synthase (EC 2.7.8.7), 3-oxoacyl-enzyme [acyl-carrier protein], 3-oxoacyl-[acyl-carrier-protein] reductase (EC 1.1.1.100), or 3-oxoacyl-[acyl-carrier-protein] synthase (EC 2.3.1.41).

In yet other embodiments, the acyl coenzyme A metabolizing or binding proteins are enzymes or proteins involved in reactions using Coenzyme A. Exemplary enzymes or proteins involved in reactions using Coenzyme A include, but are not limited to, acetate-coA ligase (EC 6.2.1.1), acetoacetyl-coA hydrolase (EC 3.1.2.11), acetoacetyl-coA: acetate coA transferase (EC 2.8.3.8), acetyl-coA acetyltransferase [thiolase] (EC 2.3.1.9), acetyl-coA acyltransferase (EC 2.3.1.16), acetyl-coA carboxylase (EC 6.4.1.2), [acetyl-coA carboxylase] phosphatase (EC 3.1.3.4), acetyl-coA ligase (EC 6.2.1.1), acyl-coA acyltransferase (EC 2.3.1.16), acyl-coA dehydrogenase (EC 1.3.99.3), acyl-coA dehydrogenase (NADP+) (EC 1.3.1.8), butyryl-coA dehydrogenase (EC 1.3.99.2), cholate-coA ligase (EC 6.2.1.7), dephospho-coA kinase (EC 2.7.1.24), enoyl-coA hydratase (EC 4.2.1.17), formyl-coA hydrolase (EC 3.1.2.10), glucan-1,4-a-glucosidase [glucoAmylase] (EC 3.2.1.3), glutaryl-coA dehydrogenase (EC 1.3.99.7), glutaryl-coA ligase (EC 6.2.1.6), 3-hydroxyacyl-coA dehydrogenase (EC 1.1.1.35), 3-hydroxybutyryl-coA dehydratase (EC 4.2.1.55), 3-hydroxybutyryl-coA dehydrogenase (EC 1.1.1.157), 3-hydroxyisobutyryl-coA hydrolase (EC 3.1.2.4), hydroxymethylglutaryl-coA lyase (EC 4.1.3.4), hydroxymethylglutaryl-coA reductase (EC 1.1.1.88), hydroxymethylglutaryl-coA reductase (NADPH) (EC 1.1.1.34), [hydroxymethylglutaryl-coA reductase (NADPH)] kinase (EC 2.7.1.109), [hydroxymethylglutaryl-coA reductase (nadph)] phosphatase (EC 3.1.3.47), hydroxymethylglutaryl-coA synthase (EC 4.1.3.5), lactoyl-coA dehydratase (EC 4.2.1.54), malonate-coA transferase (EC 2.8.3.3), malonyl-coA decarboxylase (EC 4.1.1.9), methylcrotonyl-coA carboxylase (EC 6.4.1.4), methylglutaconyl-coA hydratase (EC 4.2.1.18), methylmalonyl-coA carboxyltransferase (EC 2.1.3.1), methylmalonyl-coA decarboxylase (EC 4.1.1.41), methylmalonyl-coA epimerase (EC 5.1.99.1), methylmalonyl-coA mutase (EC 5.4.99.2), oxalate-coA transferase (EC 2.8.3.2), oxalyl-coA decarboxylase (EC 4.1.1.8), 3-oxoacid-coA transferase (EC 2.8.3.5), 3-oxoadipate coA-transferase (EC 2.8.3.6), palmitoyl-coA-enzyme palmitoyltransferase, propionate-coA ligase (EC 6.2.1.17), propionyl-coA carboxylase (EC 6.4.1.3), succinate-coA ligase (ADP-forming) (EC 6.2.1.5), succinate-coA ligase (GDP-forming) (EC 6.2.1.4), or succinate-propionate coA transferase.

In yet other embodiments, the acyl coenzyme A metabolizing or binding proteins are enzymes or proteins involved in reactions resulting in the biosynthesis or degradation of coA. Exemplary enzymes or proteins involved in reactions resulting in the biosynthesis or degradation of coA include, but are not limited to, pantothenatekinase (EC 2.7.1.33), pantothenate-B-alanine ligase (EC 6.3.2.1), phosphopantothenate-cysteine ligase (EC 6.3.2.5), pantetheine kinase (EC 2.7.1.34), pantetheine-phosphate adenylyltransferase (EC 2.7.7.3), 2-dehydropantoate reductase (EC 1.1.1.169), pantothenase (EC 3.5.1.22), pantothenoylcysteine decarboxylase (EC 4.1.1.30), phosphopantothenate-cysteine ligase (EC 6.3.2.5), phosphopantothenoylcysteine decarboxylase (EC 4.1.1.36).

In yet other embodiments, the acyl coenzyme A metabolizing or binding proteins are enzymes or proteins involved in the “mevalonate shunt,” as described in Edmond and Popjak, 1974, J. Biol. Chem. 249:66-71.

The present invention will be further understood by reference to the following non-limiting examples. The following examples are provided for illustrative purposes only and are not to be construed as limiting the invention scope of the invention in any manner.

### 6. EXAMPLES

### 6.1. Experimental

### Example 1

### 9-Hydroxy-3-(6-hydroxy-5,5-dimethylhexyl)-8,8-dimethylnonan-2-one (Compound G)

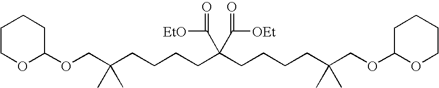

2,2-Bis-[5,5-dimethyl-6-(tetrahydropyran-2-yloxy)-hexyl]-malonic acid diethyl ester. Under nitrogen atmosphere, to a solution of 2-(6-bromo-2,2-dimethylhexyl)-tetrahydropyran (U.S. Pat. No. 6,459,003 B1; 17.6 g, 60 mmol) and diethyl malonate (4.8 g, 30 mmol) in anhydrous DMSO (145 mL) was added sodium hydride (60% dispersion in mineral oil, 2.88 g, 72 mmol) under cooling with a water bath. Tetrabutylammonium iodide (2.1 g, 3.6 mmol) was added and the mixture was stirred for 16 h at room temperature. Water (140 mL) was added carefully to the reaction mixture under cooling with water bath. The product was extracted with diethyl ether (3×60 ml). The combined organic layers were washed with water (4×50 mL) and brine (50 mL), dried over sodium sulfate, and concentrated in vacuo to give 2,2-bis-[5,5-dimethyl-6-(tetrahydropyran-2-yloxy)-hexyl]-malonic acid diethyl ester (17.3 g, 82%) as an oil. ¹H NMR (300 MHz, CDCl₃/TMS): δ (ppm): 4.41 (t, J=3.1 Hz, 2H), 4.01 (q, J=7.0 Hz, 4H), 3.82-3.70 (m, 2H), 3.50-3.30 (m, 4H), 2.87 (d, J=9.1 Hz, 2H), 1.80-1.35 (m, 16H), 1.30-0.95 (m, 18H), 0.88-0.74 (m, 12H). ¹³C NMR (75 MHz, CDCl₃/TMS): δ (ppm): 172.0, 99.1, 76.6, 61.9, 60.9, 57.6, 39.2, 34.3, 32.3, 30.7, 25.7, 25.0, 24.6, 24.6, 24.3, 19.5, 14.2.

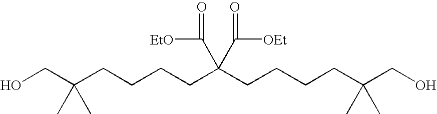

2,2-Bis-(6-hydroxy-5,5-dimethylhexyl)-malonic acid diethyl ester. A solution of 2,2-bis-[5,5-dimethyl-6-(tetrahydropyran-2-yloxy)-hexyl]-malonic acid diethyl ester (2.92 g, 5 mmol) in concentrated, aqueous HCl (2.4 mL) and water (1.6 mL) was heated to reflux for 1 h. Ethanol (8.2 ml) was added and the reaction mixture was heated to reflux for additional 3 h. The reaction mixture was diluted with water (20 mL) and extracted with diethyl ether (3×20 mL). The organic layers was washed with water (20 mL) and brine (20 mL), dried over Na₂SO₄, and concentrated to yield 2,2-bis-(6-hydroxy-5,5-dimethylhexyl)-malonic acid diethyl ester (1.74 g, 84%) as an oil. ¹H NMR (300 MHz, CDCl₃/TMS): δ (ppm): 4.13(q, J=7.2 Hz, 4H), 3.25 (s, 4H), 2.42 (s, 2H), 1.90-1.75 (m, 4H), 1.30-1.12 (m, 18H), 0.84 (s, 12H). ³C NMR (75 MHz, CDCl₃/TMS): δ (ppm): 172.0, 71.7, 60.9, 57.4, 38.2, 34.9, 32.1, 24.8, 24.0, 23.7, 14.0. HRMS (FAB, gly): Calcd for C₂₃H₄₅O₆ (MH⁺): 417.3216, found: 417.3210.

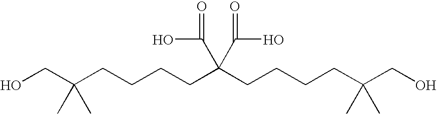

2,2-Bis-(6-hydroxy-5,5-dimethylhexyl)-malonic acid. To a stirred solution of potassium hydroxide (85%, 4.83 g, 75 mmol) in water (4.2 mL) and ethanol (15 mL) was added 2,2-bis-(6-hydroxy-5,5-dimethylhexyl)-malonic acid diethyl ester (15 g, 36.0 mmol). The reaction mixture was heated to reflux for 14 h. Ethanol was removed under reduced pressure and the remaining aqueous solution was extracted with chloroform (2×50 mL). The aqueous layer was acidified with HCl until pH 1 and extracted with diethyl ether (3×50 mL). The ethereal solution was dried over MgSO₄ and concentrated in vacuo at 80° C. to give 2,2-bis-(6-hydroxy-5,5-dimethylhexyl)-malonic acid (7.8 g, 82%) as yellow solid. Mp 178-180° C. ¹H NMR (300 MHz, CD₃OD/TMS): δ (ppm): 4.86 (s, 4H), 3.22 (s, 4H), 1.9-1.8 (m, 4H), 1.36-1.10 (m, 12H), 0.84 (s, 12H). ¹³C NMR (75 MHz, CD₃OD/TMS): δ (ppm): 176.0, 72.0, 58.7, 39.8, 36.0, 34.1, 26.5, 25.5, 24.5. HRMS (LSIMS, gly): Calcd for C₁₉H₃₇O₆ (MH⁺): 361.2590, found: 361.2582.

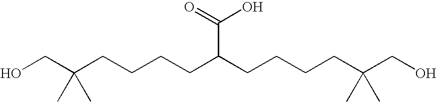

8-Hydroxy-2-(6-hydroxy-5,5-dimethylhexyl)-7,7-dimethyloctanoic acid. Using an oil-bath, 2,2-bis-(6-hydroxy-5,5-dimethylhexyl)-malonic acid (4.69 g, 13.0 mmol) was heated to 200° C. for 30 min, affording 8-hydroxy-2-(6-hydroxy-5,5-dimethylhexyl)-7,7-dimethyloctanoic acid (4.04 g, 98%) as an oil. ¹H NMR (300 MHz, CD₃OD/TMS): δ (ppm): 4.88 (s, 3H), 3.22 (s, 4H), 2.29 (m, 1H), 1.70-1.40 (m, 4H), 1.4-1.1 (m, 12H), 0.84 (s, 12H). ¹³C NMR (75 MHz, CD₃OD/TMS): δ (ppm): 180.5, 72.1, 47.1, 39.9, 36.0, 33.8, 29.7, 25.0, 24.6. HRMS (FAB, gly): Calcd for C₁₈H₃₇O₄ (MH⁺): 317.2692, found: 317.2689.

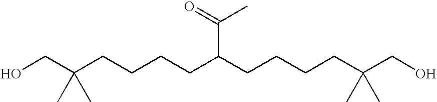

9-Hydroxy-3-(6-hydroxy-5,5-dimethylhexyl)-8,8-dimethylnonan-2-one. A solution of 8-hydroxy-2-(6-hydroxy-5,5-dimethylhexyl)-7,7-dimethyloctanoic acid (1.0 g, 3.16 mmol) in THF (40 mL) was cooled in an ice-water bath and methyl lithium (1.4 M in diethyl ether, 27 mL, 37.8 mmol) was added. The reaction was kept for 2 h at 0° C., then poured into dilute hydrochloric acid (5 mL concentrated hydrochloric acid/60 mL water). The organic layer was separated and the aqueous layer was extracted with diethyl ether (2×50 mL). The combined organic layers were dried over sodium sulfate and concentrated. The crude product (1.0 g) was purified by column chromatography (hexanes/ethyl acetate=80/20, then 50/50) to give 9-hydroxy-3-(6-hydroxy-5,5-dimethylhexyl)-8,8-dimethylnonan-2-one (0.41 g, 41%) as an oil, together with 7-(1-hydroxy-1-methylethyl)-2,2,12,12-tetramethyltridecan-1,13-diol (0.4 g, 38%, no data given here). ¹H NMR (300 MHz, CDCl₃/TMS): δ (ppm): 3.26 (s, 4H), 2.45-2.30 (m, 1H), 2.08 (s, 3H), 1.86 (br, 2H), 1.62-1.30 (m, 4H), 1.30-1.05 (m, 12H), 0.82 (s, 12H). ¹³C NMR (75 MHz, CDCl₃/TMS): δ (ppm): 213.4, 71.7, 53.2, 38.3, 34.9, 31.6, 28.7, 28.3, 23.8. HRMS (LSIMS, gly): Calcd for C₁₉H₃₉O₃ (MH⁺): 315.2899, found: 315.2866. HPLC (Alltima C₈, 250 mm×4.6 mm, 5 μm, flow rate 1.0 mL/min, acetonitrile/water=50/50, RI detection, retention time 13.68 min): 90.45%.

### Example 2

### 6-(6-Hydroxy-5,5-dimethylhexylamino)-2,2-dimethylhexan-1-ol (Compound H)

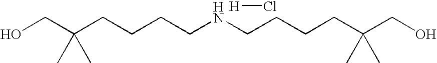

6-(6-Hydroxy-5,5-dimethylhexylamino)-2,2-dimethylhexan-1-ol hydrochloride. A mixture of 2-(6-bromo-2,2-dimethylhexyloxy)-tetrahydropyran (U.S. Pat. No. 6,459,003 B1; 15.2 g, 51.8 mmol),p-toluenesulfonamide (4.43 g, 25.9 mmol), sodium hydroxide (2.60 g, 64.75 mmol), tetrabutylammonium iodide (480 mg, 1.30 mmol), benzene (175 mL), and water (50 mL) was stirred vigorously and heated to 70° C. under N₂-atmosphere. Additional tetrabutylammonium iodide (400 mg, 1.08 mol) was added after 20 h and stirring was continued at 80° C. After a total reaction time of 44 h, the mixture was cooled to room temperature, the layers were separated, and the organic layer was extracted with water (100 mL). The organic layer was dried over MgSO₄, concentrated, and dried in vacuo to furnish crude N,N-bis-[5,5-dimethyl-6-(tetrahydropyran-2-yloxy)-hexyl]-4-methyl-benzene-sulfonamide (14.5 g) as a colorless, viscous oil. Under N₂-atmosphere, anhydrous dimethoxyethane (75 mL) was added to a mixture of sodium (2.15 g, 93.6 mmol) and naphthaline (14.8 g, 115.5 mmol). The reaction mixture was stirred for 2 h at room temperature to give a dark-green solution of sodium naphthalenide. A portion of this solution (ca. 40 mL) was added dropwise to a solution of bis-(5,5-dimethyl-6-tetrahydropyranyloxyhexyl)-p-toluenesulfonamide (7.0 g, 11.7 mmol) in anhydrous dimethoxyethane (200 mL) at −78° C. until a greenish-colored solution persisted. After additional 15 min, the reaction mixture was hydrolized with saturated NaHCO₃ solution (20 mL) and warmed to room temperature. Potassium carbonate (100 g) was added and the reaction mixture was stirred for 1.5 h. The solids were removed by filtration and washed with diethyl ether (2×200 mL). The filtrate was dried over sodium sulfate and concentrated to give crude bis-(5,5-dimethyl-6-tetrahydropyranyloxy-hexyl)-amine (13.5 g) as an oil. The obtained oil (13.5 g) was dissolved in methanol (100 mL), concentrated HCl (10 mL) was added, and the reaction mixture was heated to reflux under an N₂-atmosphere for 2 h. After cooling to room temperature, water (200 mL) was added and the non-salts were removed by extraction with CH₂Cl₂ (3×100 mL). The pH of the aqueous layer was adjusted to 11 with solid Na₂CO₃. The aqueous layer was extracted with CH₂Cl₂ (3×100 mL). The combined organic layers were dried over Na₂SO₄, and concentrated in vacuo to give the free amine as a red oil. This oil was dissolved in ethanol (20 mL) and acidified with concentrated HCl (2 mL) to pH 1. The solvents were removed in high vacuo, affording 6-(6-hydroxy-5,5-dimethylhexylamino)-2,2-dimethylhexan-1-ol hydrochloride (1.45 g, 37% over three steps) as a reddish glass. ¹H NMR (300 MHz, CD₃OD/TMS): δ (ppm): 3.24 (s, 4H), 3.00 (m, 4H), 1.70 (m, 4H), 1.48-1.22 (m, 8H), 0.88 (s, 12H). ¹³C NMR (75 MHz, CD₃OD/TMS): 6 (ppm): 71.66, 49.06, 39.34, 36.02, 28.25, 24.65, 22.25. HRMS (LSIMS, gly): Calcd for C₁₆H₃₆NO₂: 274.2746, found: 274.2746. GC (Alltech AT-5, 15 m×0.53 μm, 1.2 mm film, retention time 18.2 min): 95.5%.

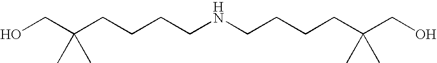

6-(6-Hydroxy-5,5-dimethylhexylamino)-2,2-dimethylhexan-1-ol. 6-(6-Hydroxy-5,5-dimethyl-hexylamino)-2,2-dimethylhexan-1-ol hydrochloride (7.68 g, 24.78 mmol) was extracted with 10% aqueous NaOH solution (100 mL) and dichloromethane (80 mL). The layers were separated and the aqueous layer was extracted with dichloromethane (2×80 mL). The combined organic layers were washed with saturated NaCl solution (50 mL), dried over Na₂SO₄, concentrated in vacuo, and dried in high vacuo to afford 6-(6-hydroxy-5,5-dimethylhexylamino)-2,2-dimethylhexan-1-ol (5.55 g, 82%) as an orange, viscous oil. ¹H NMR (300 MHz, CDCl₃/TMS): δ (ppm): 3.27 (s, 4H), 3.1-2.4 (br, OH, NH), 2.60 (t, 4H, J=7.1 Hz), 1.48 (m, 4H), 1.25 (m, 8H), 0.85 (s, 12H). ¹³C NMR (75 MHz, CDCl₃/TMS): δ (ppm): 71.15, 49.62, 38.05, 35.13, 30.35, 24.29, 21.36. HRMS (LSIMS, gly): Calcd for C₁₆H₃₆NO₂ (MH⁺): 274.2746, found: 274.2746.

### Example 3

### 6-1Hydroxy-(6-hydroxy-5,5-dimethylhexyl)-amino]-2,2-dimethylhexan-1-ol (Compound I)

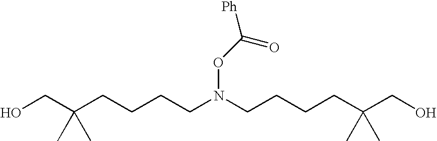

6-[Benzoyloxy-(6-hydroxy-5,5-dimethylhexyl)-amino]-2,2-dimethylhexan-1-ol. Under Ar-atmosphere, to a stirred suspension of 6-(6-hydroxy-5,5-dimethylhexylamino)-2,2-dimethylhexan-1-ol (2.62 g, 9.58 mmol) and disodium hydrogen phosphate (6.95 g, 48.93 mmol) in methyl tert-butyl ether (MTBE, 50 mL) was added dropwise over 45 min a solution of benzoyl peroxide (2.55 g, 10.54 mmol) in MTBE (90 mL) at room temperature. The mixture was heated to 45° C. for 17 h, cooled to room temperature, diluted with MTBE (100 mL), and extracted with 10% sodium carbonate solution (2×100 mL) and brine (50 mL). The organic layer was dried over magnesium sulfate and concentrated in vacuo. The residue was purified by flash chromatography (silica, hexanes/ethyl acetate=50/50) to afford 6-[benzoyloxy-(6-hydroxy-5,5-dimethylhexyl)-amino]-2,2-dimethylhexan-1-ol (2.24 g, 59%) as a viscous, slightly yellowish oil. ¹H NMR (300 MHz, CDCl₃/TMS): δ (ppm): 8.01 (m, 2H), 7.97 (m, 1H), 7.44 (m, 2H), 3.29 (s, 4H), 2.98 (t, 4H, J=7.3 Hz), 2.62 (s, 2H), 1.56 (m, 4H), 1.42-1.16 (m, 8H), 0.83 (s, 12H). ¹³C NMR (75 MHz, CDCl₃/TMS): δ (ppm): 165.92, 133.17, 129.56, 129.17, 128.51, 71.20, 59.33, 37.97, 35.04, 27.46, 24.20, 21.28. HRMS (LSIMS, gly): Calcd for C₂₃H₄₀NO₄ (MH⁺): 394.2957, found: 394.2954.

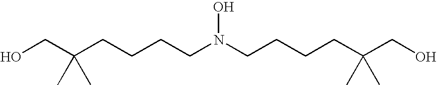

6-1Hydroxy-(6-hydroxy-5,5-dimethylhexyl)-amino]-2,2-dimethylhexan-1-ol. Under Ar-atmosphere, to a solution of 6-[benzoyloxy-(6-hydroxy-5,5-dimethylhexyl)-amino]-2,2-dimethylhexan-1-ol (2.0 g, 5.08 mmol) in anhydrous methanol (20 mL) was added a solution of sodium methoxide in anhydrous methanol (0.5 M, 20.4 mL, 10.16 mmol) at room temperature. The mixture was stirred for 4 h, diluted with saturated NH₄Cl solution (200 mL), and extracted with dichloromethane (2×50 mL). The combined organic layers were washed with saturated NaCl solution (100 mL), dried over MgSO₄, concentrated in vacuo, and dried in high vacuo to give the crude product (1.6 g). This residue was purified by flash chromatography (silica, hexanes/ethyl acetate=25/75) to afford 6-[hydroxy-(6-hydroxy-5,5-dimethylhexyl)-amino]-2,2-dimethylhexan-1-ol (710 mg, 48%) as a white solid. Crystallization from methyl tert-butyl ether/hexanes (10 mL, 50/50) at −5° C. furnished the product (620 mg, 42%) in form of white crystals. Mp 73° C. ¹H NMR (300 MHz, CDCl₃/TMS): δ (ppm): 3.30 (s, 4H), 2.67 (t, 4H, J=7.1 Hz), 1.58 (m, 4H), 1.27 (m, 4H), 0.85 (s, 12H). ¹³C NMR (75 MHz, CDCl₃/TMS): δ (ppm): 71.59, 60.57, 38.31, 35.21, 27.85, 24.29, 21.68. HRMS (LSIMS, gly): Calcd for C₁₆H₃₆NO₃ (MH⁺): 290.2695, found: 290.2676.

### Example 4

### 7-Hydroxy-6,6-dimethylheptanoic acid (6-hydroxy-5,5-dimethylhexyl)-amide (Compound J)

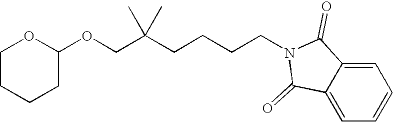

2-[5,5-Dimethyl-6-(tetrahydropyran-2-yloxy)-hexl]-isoindole-1,3-dione. Under N₂ atmosphere, potassium phthalimide (49.1 g, 265 mmol) was added to a stirred solution of 2-(6-bromo-2,2-dimethylhexyloxy)-tetrahydropyran (U.S. Pat. No. 6,459,003 B1; 70.7 g, 241 mmol) in DMF (150 mL, dried over 4-Å molecular sieves) at room temperature. The suspension was heated to 80-95° C. for 3 h. The reaction mixture was cooled to room temperature, diluted with water (500 mL), and extracted with diethyl ether (2×250 mL, 1×100 mL). The combined organic layers were washed with saturated NaCl solution (100 mL), dried over MgSO₄, concentrated in vacuo, and dried in high vacuo to furnish 2-[5,5-dimethyl-6-(tetrahydropyran-2-yloxy)-hexyl ]-isoindole-1,3-dione (78.4 g, 90%) as a yellowish oil. ¹H NMR (300 MHz, CDCl₃): δ (ppm): 7.84 (dd, 2H, J=5.4, 3.1 Hz), 7.71 (dd, 2H, J=5.4, 3.1 Hz), 4.53 (t, 1H, J=2.9 Hz), 3.81 (m, 1H), 3.68 (t, 2H, J=7.3 Hz), 3.48 (m, 1H), 3.46 (d, 1H, J=9.2 Hz), 2.97 (d, 1H, J=9.2 Hz), 2.97 (d, 1H, J=9.2), 1.90-1.42 (m, 9H), 1.31 (m, 3H), 0.89 (s, 3H), 0.88 (s, 3H). ¹³C NMR (75 MHz, CDCl₃/TMS): δ (ppm): 168.42, 133.89, 132.26, 123.18, 99.09, 76.45, 61.88, 38.95, 38.13, 34.25, 30.70, 29.59, 25.65, 24.60, 21.44, 19.48. HRMS (LSIMS, gly): Calcd for C₁₆H₂₂NO₃ (MH⁺−DHP): 276.1600, found: 276.1597.

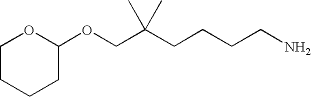

5,5-Dimethyl-6-(tetrahydropyran-2-yloxy)-hexylamine. A solution of hydrazine hydrate in water (85% w/w, 17.3 g, 294 mmol) was added dropwise to a solution of 2-[5,5-dimethyl-6-(tetrahydropyran-2-yloxy)-hexyl]-isoindole-1,3-dione (78.0 g, 217 mmol) in ethanol (400 mL) at room temperature. The reaction mixture was heated to reflux for 1 h, then cooled to room temperature. The precipitate was removed by filtration and washed with ethanol (2×100 mL). The filtrate was concentrated to a volume of ca. 100 mL. Additional precipitate was filtered off and washed with diethyl ether (4×100 mL). The combined organic layers were washed with saturated NaCl solution (3×75 mL), dried over MgSO₄, concentrated in vacuo, and dried in high vacuo to give 5,5-dimethyl-6-(tetrahydropyran-2-yloxy)-hexylamine (29.0 g, 58%) as a yellowish oil. ¹H NMR (300 MHz, CDCl₃/TMS): 6 (ppm): 4.58 (s, 2H, OH), 4.54 (t, 1H, J=3.5 Hz), 3.82 (m, 1H), 3.49 (m, 4H), 3.46 (d, 1H, J=9.1 Hz), 2.98 (d, 1H, J=9.1 Hz), 2.78 (t, 2H, J=7.3 Hz), 1.94-1.44 (m, 8H), 1.40-1.20 (m, 4H), 0.89 (s, 6H). ¹³C NMR (75 MHz, CDCl₃/TMS): δ (ppm): 99.08, 76.34, 61.89, 41.18, 38.91, 34.15, 32.29, 30.63, 25.53, 24.49, 21.17, 19.44. HRMS (LSIMS, nba): Calcd for C₁₃H₂₈NO₂ (MH⁺): 230.2120, found: 230.2123.

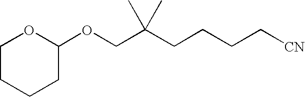

6,6-Dimethyl-7-(tetrahydropyran-2-yloxy)-heptanonitrile. Under N₂-atmosphere dry sodium cyanide (2.1 g, 42.9 mmol) was added to DMSO (100 mL) and the mixture was heated to 90° C. A solution of 2-(6-bromo-2,2-dimethylhexyloxy)-tetrahydropyran (10.0 g, 34.1 mmol) in DMSO (50 mL) was added dropwise. The mixture was heated to 105° C. for 1 h and stirred overnight at room temperature. The mixture was poured into water (400 mL) and extracted with CHCl₃ (3×200 mL). The extracts were washed with brine (4×200 mL) and dried over CaCl₂. The solvent was removed under reduced pressure to give 6,6-dimethyl-7-(tetrahydropyran-2-yloxy)-heptanonitrile (7.5 g, 88%) as a colorless liquid. ¹H NMR (300 MHz, CDCl₃/TMS): 8 (ppm): 4.45 (s, 1H), 3.79 (m, 1H), 3.45 (m, 1H), 3.42 (d, 1H, J=9.0 Hz), 2.94 (d, J=9.0 Hz, 1H), 2.30 (t, J=6.0 Hz, 2H), 1.85-1.15 (m, 12H), 0.82 (s, 6H). ³C NMR (75 MHz, CDCl₃/TMS): δ (ppm): 120.6, 99.9, 77.0, 62.8, 39.1, 34.9, 31.4, 27.0, 26.3, 25.3, 25.2, 23.9, 20.2, 17.8. HRMS (LSIMS, nba): Calcd for C₁₄H₂₆NO₂ (MH⁺) 240.1964, found: 240.1941.

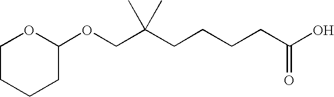

6,6-Dimethyl-7-(tetrahydropyran-2-yloxy)-heptanoic acid. A solution of 6,6-dimethyl-7-(tetrahydropyran-2-yloxy)-heptanonitrile (8.0 g, 33.5 mmol) and sodium hydroxide (4.80 g, 120 mmol) in ethanol (80 mL) and water (80 mL) was heated to reflux for 24 h. The reaction mixture was concentrated to a volume of ca. 80 mL and extracted with CH₂Cl₂ (300 mL). Additional water (500 mL) and CH₂Cl₂ (500 mL) were added for better separation of layers. The aqueous layer was acidified with 1 N aqueous HCl (100 mL) to pH 1 and extracted with CH₂Cl₂ (2×400 mL). The combined organic layers were washed with brine (200 mL), dried over MgSO₄, concentrated in vacuo and dried in high vacuo to furnish 6,6-dimethyl-7-(tetrahydropyran-2-yloxy)-heptanoic acid (8.0 g, 74%) as a colorless oil. ¹H NMR (300 MHz, CDCl₃/TMS): δ (ppm): 9.75 (m br, 1H), 4.55 (m br, 1H), 3.85 (m, 1H), 3.50 (m, 1H), 3.45 (d, 1H, J=9.0 Hz), 2.95 (d, J=9.0 Hz, 1H), 2.35 (t, J=6.0 Hz, 2H), 1.90-1.45 (m, 8H), 1.30 (m, 4H), 0.90 (s, 6H). ¹³C NMR (75 MHz, CDCl₃/TMS): δ (ppm): 179.74, 99.17, 76.56, 61.94, 39.01, 34.30, 30.74, 25.74, 25.66, 24.70, 23.64, 19.47. HRMS (LSIMS, gly): Calcd for C₁₄H₂₇O₄ (MH⁺): 259.1909, found: 259.1898.

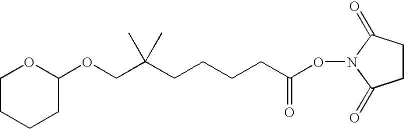

6,6-Dimethyl-7-(tetrahydropyran-2-yloxy)-heptanoic acid 2,5-dioxopyrrolidinyl ester. Under nitrogen atmosphere, to a solution of 6,6-dimethyl-7-(tetrahydropyran-2-yloxy)-heptanoic acid (3.0 g, 11.70 mmol) in CH₂Cl₂ (500 mL) was added N-hydroxysuccinimide (1.34 g, 11.70 mmol) and DCC (2.40 g, 1.17 mmol). The reaction mixture was stirred at room temperature for 4 h. The dicyclohexylurea (DCU) formed was filtered off and the solution was concentrated in vacuo. The residue was dissolved in diethyl ether (100 mL) and the insoluble solid (DCU) was removed by filtration. The filtrate was concentrated in vacuo and dried in high vacuo to furnish 6,6-dimethyl-7-(tetrahydropyran-2-yloxy)-heptanoic acid 2,5-dioxopyrrolidinyl ester (3.10 g, 75%) as a light yellowish oil. ¹H NMR (300 MHz, CDCl₃/TMS): δ (ppm): 4.50 (m, 1H), 3.75 (m, 1H), 3.35 (m, 2H), 2.90 (d, J=9.0 Hz, 1H), 2.70 (s, 4H), 2.57 (t, J=6.0, 2H), 1.80-1.05 (m, 12H), 0.90 (s, 6H). ¹³C NMR (75 MHz, CDCl₃/TMS): δ (ppm): 169.6, 169.1, 99.5, 76.8, 62.3, 39.1, 34.6, 31.4, 31.0, 25.9, 24.9, 24.8, 23.7, 19.8. HRMS (LSIMS): Calcd for C₁₈H₃₀O₆N (MH⁺): 356.2073, found: 356.2101.

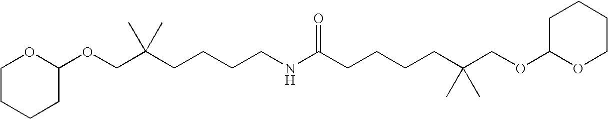

6,6-Dimethyl-7-(tetrahydropyran-2-yloxy)-heptanoic acid [5,5-dimethyl-6-(tetrahydro-pyran-2-yloxy)-hexyl]-amide. To a solution of 5,5-dimethyl-6-(tetrahydropyran-2-yloxy)-hexylamine (1.0 g, 4.37 mmol) in CH₂Cl₂ (600 mL) was added dropwise a solution of 6,6-dimethyl-7-(tetrahydropyran-2-yloxy)-heptanoic acid 2,5-dioxopyrrolidinyl ester (1.55 g, 4.37 mmol) in CH₂Cl₂ (200 mL). The reaction mixture was stirred for 48 h at room temperature. The solvent was removed in vacuo. The residue was dissolved in diethyl ether and the solution filtered to remove leftover DCU from the previous step. The filtrate was concentrated in vacuo and purified by flash chromatography (silica gel, ethyl acetate/hexanes=1/2) to furnish 6,6-dimethyl-7-(tetrahydropyran-2-yloxy)-heptanoic acid [5,5-dimethyl-6-(tetrahydro-pyran-2-yloxy)-hexyl]-amide (1.56 g, 76%) as a colorless oil. ¹H NMR (300 MHz, CDCl₃/TMS): δ (ppm): 5.50 (s, 1H), 4.50 (s, 2H), 3.75 (m, 2H), 3.40(m, 4H), 3.15 (m, 2H), 2.95 (d, J=9 Hz, 2H), 2.12 (t, J=7 Hz, 2H), 1.90-1.0 (m, 24H), 0.85 (s, 12H). ¹³C NMR (75 MHz, CDCl₃/TMS): δ (ppm): 173.17, 99.27, 99.20, 76.50, 76.43, 62.12, 62.01, 39.44, 38.97, 38.85, 36.85, 34.20, 30.72, 30.52, 26.83, 25.60, 24.54, 23.70, 21.29, 19.60, 19.53. HRMS (LSIMS): Calcd for C₂₇H₅₂O₅N (MH⁺): 470.3845, found: 470.3839.

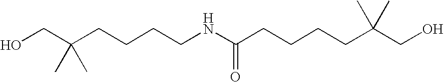

7-Hydroxy-6,6-dimethylheptanoic acid (6-hydroxy-5,5-dimethylhexyl)-amide. To a solution of 6,6-dimethyl-7-(tetrahydropyran-2-yloxy)-heptanoic acid [5,5-dimethyl-6-(tetrahydro-pyran-2-yloxy)-hexyl]-amide (1.41 g, 3.0 mmol) in methanol (50 mL) was added aqueous HCl (9 mL, 37%). The reaction mixture was heated to reflux for 2 h, cooled to room temperature, diluted with water (50 mL), and concentrated in vacuo to a volume of ca. 60 mL. The solution was extracted with CH₂Cl₂ (3×100 mL). The combined organic layers were washed with saturated NaHCO₃ solution (3×100 mL) and brine (50 mL), dried over MgSO₄, and concentrated in vacuo. The residual oil was purified by flash chromatography (silica, ethyl acetate/hexanes=1/2, followed by methylene chloride/methanol=10/1) to furnish 7-hydroxy-6,6-dimethylheptanoic acid (6-hydroxy-5,5-dimethylhexyl)-amide (0.77 g, 85%) as a yellowish oil. ¹H NMR (300 MHz, CDCl₃/TMS): δ (ppm): 6.05 (m, 1H), 3.28 (s, 4H), 3.25 (m, 2H), 2.58 (br, 2H), 2.19 (t, J=7.0 Hz, 2H), 1.61 (m, 2H), 1.48 (m, 2H), 1.25 (m, 8H), 0.85 (s, 12H). ¹³C NMR (75 MHz, CDCl₃/TMS): δ (ppm): 173.80, 71.35, 71.15, 39.10, 37.99, 37.82, 36.60, 35.17, 30.55, 26.69, 24.40, 24.30, 23.41, 20.80. HRMS (LSIMS, gly): Calcd for C₁₇H₃₆NO₃ (MH⁺): 302.2695, found: 302.2723. HPLC (Alltima C₈, 250 mm×4.6 mm, 5 μm, flow rate 1.0 mL/min, acetonitrile/water 10/90, RI detection, retention time 1.95 min): 97.0%.

### Example 5

### 7-Hydroxy-6,6-dimethylheptanoic acid 6-hydroxy-5,5-dimethylhexyl ester (Compound K).

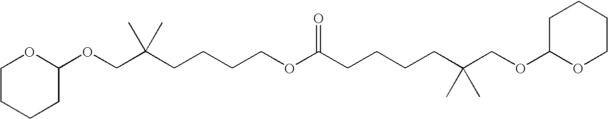

6,6-Dimethyl-7-(tetrahydropyran-2-yloxy)-heptanoic acid 5,5-dimethyl-6-(tetrahydro-pyran-2-yloxy)-hexyl ester. A mixture of 5-5-dimethyl-6-(tetrahydropyran-2-yloxy)-hexan-1-ol (U.S. Pat. No. 6,459,003 B1; 8.1 g, 35.2 mmol), 6,6-dimethyl-7-(tetrahydropyran-2-yloxy)-heptanoic acid (10.0 g, 38.8 mmol), DCC (8.8 g, 42.7 mmol), and DMAP (1.1 g, 9.0 mmol) in CH₂Cl₂ (600 mL) under N₂ atmosphere was stirred at room temperature for 18 h. The precipitated dicyclohexyl urea was removed by filtration. The filtrate was concentrated in vacuo and the residue purified by column chromatography (silica, hexanes/ethyl acetate=5/1), affording 6,6-dimethyl-7-(tetrahydropyran-2-yloxy)-heptanoic acid 5,5-dimethyl-6-(tetrahydropyran-2-yloxy)-hexyl ester (11.0 g, 66%) as an oil. ¹H NMR (300 MHz, CDCl₃/TMS): δ (ppm): 4.50 (s, 2H), 4.08 (t, J=7.5 Hz, 2H), 3.75 (m, 2H), 3.40 (m, 4H), 2.95 (m, 2H), 2.25 (t, J=7.5 Hz, 2H), 1.90-1.0 (m, 24H), 0.85 (s, 12H). ¹³C NMR (75 MHz, CDCl₃/TMS): δ (ppm): 174.46, 99.64, 77.00, 64.90, 62.43, 39.57, 35.46, 34.96, 31.21, 30.10, 26.93, 25.99, 24.26, 24.12, 20.94, 19.99. HPLC (Alltima C8, 250 mm x 4.6 mm, acetonitrile/HOAc-TEA buffer (4 mL/L HOAc, 8 mL/L TEA) 60/40, flow rate 1.0 mL/ml, UV detection, retention time 12.75 min): 56.68%.

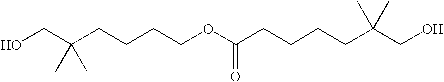

7-Hydroxy-6,6-dimethylheptanoic acid 6-hydroxy-5,5-dimethylhexyl ester. A solution of 6,6-dimethyl-7-(tetrahydropyran-2-yloxy)-heptanoic acid 5,5-dimethyl-6-(tetrahydropyran-2-yloxy)-hexyl ester (10.0 g, 21.28 mmol) in acetic acid/THF/water (4/2/1, 437.5 mL) was heated to 45° C. for 4 h. The solution was concentrated in vacuo. Diethyl ether (300 mL) was added to the crude product and the solids (dicyclohexyl urea) were removed by filtration. The filtrate was concentrated in vacuo and purified by flash chromatography (silica, ethyl acetate/hexanes=1/2) to furnish 7-hydroxy-6,6-dimethylheptanoic acid 6-hydroxy-5,5-dimethylhexyl ester (2.5 g, 39%) as an oil. ¹H NMR (300 MHz, CDCl₃/TMS): δ (ppm): 4.08 (t, J=6.6 Hz, 2H), 3.29 (s, 4H), 2.29 (m, 2H), 1.61 (m, 4H), 1.26 (m, 8H), 0.87 (s, 6H) 0.86 (s, 6H). ¹³C NMR (75 MHz, CDCl₃/TMS): δ (ppm): 174.22, 71.71, 64.39, 38.26, 35.10, 34.40, 29.62, 25.92, 23.98, 23.50, 20.38. HRMS (LSIMS, gly): Calcd for C₁₇H₃₅O₄ (MH⁺): 303.2535, found: 303.2528. HPLC (Alltima C₈, 250 mm×4.6 mm, 5 μL, methanol/water 50/50, flow rate 1.0 mL/min, retention time 9.70 min, RI detection): 97.6%.

### Example 6

### 7-Hydroxy-6,6-dimethylheptanoic acid 6-hydroxy-5,5-dimethylhexyl ester (Compound L)

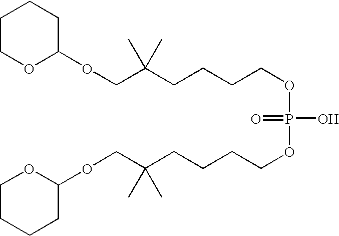

Phosphoric acid bis-15,5-dimethyl-6-(tetrahydropyran-2-yloxy)-hexyl]-ester. Phosphorus oxychloride (4.06 g, 2.5 mL, 26.48 mmol) was added dropwise to a solution of 5-5-dimethyl-6-(tetrahydropyran-2-yloxy)-hexan-1-ol (U.S. Pat. No. 6,459,003 BI; 12.2 g; 52.96 mmol) and triethylamine (5.36 g, 7.4 mL, 52.96 mmol) in anhydrous diethyl ether (200 mL) at room temperature under N₂-atmosphere. The reaction mixture was stirred for 17 h. The ammonium salts were removed by filtration and washed with diethyl ether (100 mL). The filtrate was concentrated in vacuo to give a yellowish oil (15.0 g). To a solution of this oil in water (100 mL) and acetonitrile (100 mL) was added KHCO₃ (13.3 g, 133 mmol) and the reaction mixture was stirred at room temperature for 3.5 h. The reaction mixture was diluted with water (250 mL) and extracted with diethyl ether (250 mL). The aqueous layer was acidified with concd HCl (7 mL) to pH 1 and then extracted with diethyl ether (2×250 mL). The combined organic phases were washed with saturated NaCl solution (100 mL), dried over MgSO₄, concentrated in vacuo, and dried in high vacuo to give crude phosphoric acid bis-[5,5-dimethyl-6-(tetrahydropyran-2-yloxy)-hexyl]-ester (4.1 g, 7.84 mmol, ca. 30%) as a viscous oil, which was used for the next step without further purification. ¹H NMR (300 MHz, CDCl₃/TMS): δ (ppm): 4.54 (t, 2H, J=3.3 Hz), 4.02 (m, 4H), 3.82 (m, 2H), 3.50 (m, 2H), 3.45 (d, 2H, J=9.2 Hz), 2.98 (d, 2H, J=9.2 Hz), 1.92-1.20 (m, 24H), 0.89 (s, 6H), 0.88 (s, 6H). ¹³C NMR (75 MHz, CDCl₃/TMS): δ (ppm): 99.33; 76.64, 67.76 (J=6 Hz), 62.14, 39.00, 34.40, 31.28 (J=7 Hz), 30.82, 25.72, 24.67, 20.10, 19.61.

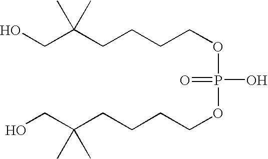

Phosphoric acid bis-(5,5-dimethyl-6-hydroxyhexyl)-ester. A solution of crude phosphoric acid bis-[5,5-dimethyl-6-(tetrahydropyran-2-yloxy)-hexyl]-ester (4.0 g; 7.65 mmol) in methanol (100 mL) and conced HCl (10 mL) was heated to reflux for 2 h. The solution was diluted with water (200 mL) and concentrated under reduced pressure to a volume of ca. 100 mL. This aqueous phase was extracted with CH₂Cl₂ (3×100 mL). The combined organic layers were extracted with saturated NaHCO₃ solution (2×50 mL). The combined aqueous layers were acidified with concd HCl (10 mL) to pH 1 and extracted with CH₂Cl₂ (3×75 mL). The combined organic layers were dried over MgSO₄, concentrated in vacuo, and dried in high vacuo to give phosphoric acid bis-(5,5-dimethyl-6-hydroxyhexyl)-ester (1.73 g, 4.88 mmol, 9% over both steps) as a viscous oil. ¹H NMR (300 MHz, CDCl₃/TMS): δ (ppm): 6.18 (m br, 3H), 4.05 (m, 4H), 3.33 (s, 4H), 1.48-1.22 (m, 8H), 0.87 (s, 12H). ¹³C NMR (75 MHz, CDCl₃/TMS): δ (ppm): 71.15, 67.29 (J=6 Hz), 37.70, 35.14, 30.94 (J=7 Hz), 24.38, 19.76. HRMS (LSIMS, gly): Calcd for C₁₆H₃₆PO₆ (MH⁺): 355.2250, found: 355.2245.

### Example 7

### (6-Hydroxy-5,5-dimethylhexyl)-carbamic acid 6-hydroxy-5,5-dimethylhexyl ester (Compound M)

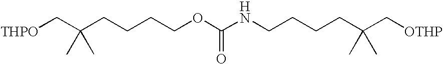

[5,5-Dimethyl-6-(tetrahydropyran-2-yloxy)-hexyl]-carbamic acid 5,5-dimethyl-6-(tetra-hydropyran-2-yloxy)-hexyl ester. To a solution of 5-5-dimethyl-6-(tetrahydropyran-2-yloxy)-hexan-1-ol (U.S. Pat. No. 6,459,003 B1; 4.0 g, 17.4 mmol) and 1,1′-carbonydiimidazole (3.52 g, 21.7 mmol) in anhydrous CH₂Cl₂ (200 mL) was added 4-dimethylaminopyridine (0.42 g, 3.5 mmol) at room temperature under N₂ atmosphere. The reaction mixture was stirred for 2 h at room temperature and concentrated in vacuo to furnish a yellowish oil (5 g). A portion of this crude oil (2.7 g) was dissolved in anhydrous CH₃CN (160 mL) and 5,5-dimethyl-6-(tetrahydropyran-2-yloxy)-hexylamine (8.25 g, 36.15 mmol) in anhydrous CH₃CN (20 mL) was added dropwise. The reaction mixture was stirred for 24 h at room temperature. The solution was washed with 15% aqueous citric acid (2×75 mL) and 1% aqueous HCl (100 mL). The combined organic layers were dried over MgSO₄ and concentrated in vacuo to afford [5,5-dimethyl-6-(tetrahydropyran-2-yloxy)-hexyl]-carbamic acid 5,5-dimethyl-6-(tetra-hydropyran-2-yloxy)-hexyl ester (3.05 g, 67%) as an oil. ¹H NMR (300 MHz, CDCl₃/TMS): δ (ppm): 4.75 (s, 1H), 4.48 (m, 2H), 3.99 (t, J=6.6 Hz, 2H), 3.77 (m, 2H), 3.42 (m, 4H), 3.11 (m, 2H), 2.94 (d, J=9.0 Hz, 2H), 1.78-1.46 (m, 16H), 1.22 (m, 8H), 0.83 (s, 12H). ¹³C NMR (75 MHz, CDCl₃/TMS): δ (ppm): 156.94, 99.38, 99.16, 76.63, 76.45, 64.98, 62.28, 62.05, 40.98, 39.17, 38.90, 34.39, 30.85, 30.14, 25.72, 24.79, 24.68, 21.18, 20.51, 19.71, 19.60. HRMS (LSIMS, nba): Calcd for C₂₇H₅₂NO₆ (MH⁺): 486.3795, found: 486.3775.

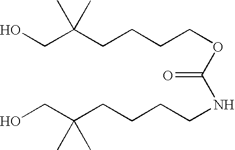

(6-Hydroxy-5,5-dimethylhexyl)-carbamic acid 6-hydroxy-5,5-dimethylhexyl ester. A solution of [5,5-dimethyl-6-(tetrahydropyran-2-yloxy)-hexyl]-carbamic acid 5,5-dimethyl-6-(tetrahydropyran-2-yloxy)-hexyl ester (7.35 g, 15.15 mmol) in acetic acid/THF/water (236 mL/118 mL/59 mL) was heated to 45° C. for 24 h. The reaction mixture was poured into ice-water (200 g) and extracted with CH₂Cl₂ (3×100 mL). The organic layers were washed with saturated NaHCO₃ solution (2×100 mL) and brine (150 mL), dried over MgSO₄, and concentrated in vacuo. The residue was purified by flash chromatography (silica gel, hexanes, then ethyl acetate/hexanes=1/10, 1/2, and 1/1), affording (6-hydroxy-5,5-dimethylhexyl)-carbamic acid 6-hydroxy-5,5-dimethylhexyl ester (3.65 g, 76%) as an oil. ¹H NMR (300 MHz, CDCl₃/TMS): δ (ppm): 4.95 (br, 1H), 4.08 (t, J=6.3 Hz, 2H), 3.25 (s, 4H), 3.15 (m, 2H), 2.17 (br, 2H), 1.53 (m, 2H), 1.41 (m, 2H), 1.21 (m, 8H), 0.81 (s, 12H). ¹³C NMR (75 MHz, CDCl₃/TMS): δ (ppm): 157.29, 71.61, 71.40, 64.83, 40.71, 38.22, 37.99, 35.19, 30.97, 30.07, 24.21, 24.12, 20.92, 20.31. HRMS (LSIMS, gly): Calcd for C₁₇H₃₆NO₄ (MH⁺): 318.2644, found: 318.2663. HPLC (C-18, 250 mm×4.6 mm, acetonitrile/water 60/40, flow rate 1.0 mL/min, RI detection, retention time 4.23 min): 99.0%.

### Example 8

### Phosphoric acid mono-16-(5,5-dimethyl-6-phosphonooxyhexyloxy)-2,2-dimethylhexyl] ester, tetraammonium salt (Compound A)

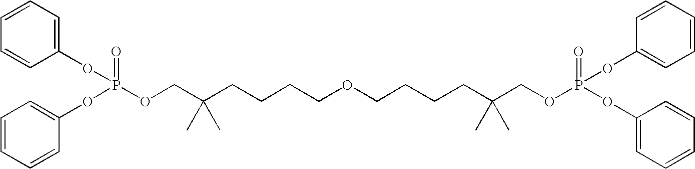

Phosphoric acid 13-(diphenyl-phosphoryloxy)-2,2,12,12-tetramethyl-7-oxa-tridecanyl ester diphenyl ester. Under inert gas atmosphere, to a stirred solution of 6-(6-hydroxy-5,5-dimethylhexyloxy)-2,2-dimethylhexan-1-ol (5.38 g, 18.7 mmol) and 4-dimethylaminopyridine (DMAP, 0.11 g, 0.9 mmol) in pyridine (60 mL) at 0° C. was added dropwise a solution of diphenyl chlorophosphate (10.32 g, 38.0 mmol) in dichloromethane, while maintaining the temperature between 0 and 10° C. The mixture was stirred at room temperature for 20 h, then poured into a mixture of 2 N HCl (400 mL), ice (100 g) and dichloromethane (140 mL). The aqueous layer was extracted with dichloromethane (100 mL) and the combined organic layers were washed with saturated NaHCO₃ solution (100 mL) and saturated NaCl solution (100 mL), dried over MgSO₄, and concentrated in vacuo to give the crude product as a colorless oil (14.0 g). The crude product was purified by column chromatography (silica; hexanes/ethyl acetate=3/1) to afford phosphoric acid 13-(diphenyl-phosphoryloxy)-2,2,12,12-tetramethyl-7-oxa-tridecanyl ester diphenyl ester (11.9 g, 87%) as a colorless oil. ¹H NMR (300 MHz, CDCl₃/TMS): δ (ppm): 7.33 (t, 8H, J=6.9 Hz), 7.24-7.15 (m, 12H), 3.92 (d, 4H, J=4.8 Hz), 3.34 (t, 4H, J=6.6 Hz), 1.51-1.46 (m, 4H), 1.26-1.24 (m, 8H), 0.89 (s, 12H). ¹³C NMR (75 MHz, CDCl₃/TMS): δ (ppm): 150.8 (d, J=6.9 Hz), 129.9, 125.5, 120.2 (d, J=5.0 Hz), 77.1 (d, J=7.5 Hz), 70.9, 38.4, 34.8 (d, J=8.0 Hz), 30.6, 23.9, 20.5. HRMS (LSIMS, nba): Calcd for C₄₀H₅₃O₉P₂ (MH⁺): 739.3164, found: 739.3210.

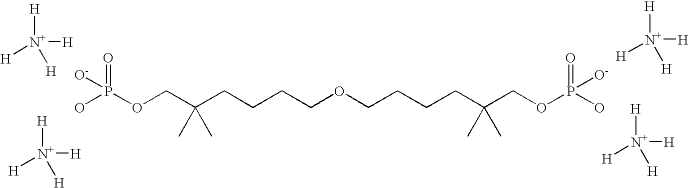

Phosphoric acid mono-16-(5,5-dimethyl-6-phosphonooxyhexvloxy)-2,2-dimethylhexyl] ester, tetraammonium salt. A 5-L three-necked flask equipped with mechanic stirrer, dry-ice condenser and argon outlet adapter was charged with THF (650 mL) and liquid ammonia (2.2 L) was condensed at −60° C. Lithium wire (6×4 cm, 49 mg/cm, 170 mmol) was dissolved under stirring and a deep blue solution was formed. To this solution was added dropwise a solution of phosphoric acid 13-(diphenyl-phosphoryloxy)-2,2,12,12-tetramethyl-7-oxatridecanyl ester diphenyl ester (62 g, 82 mmol) in THF (150 mL). After decolorization, the addition was stopped and more lithium wire [4 cm x 40, 49 mg/cm, 1130 mmol; total 184 cm, 49 mg/cm, 1300 mmol) was added in 10 portions until the blue color was restored. The addition of phosphoric acid 13-(diphenylphosphoryloxy)-2,2,12,12-tetramethyl-7-oxatridecanyl ester diphenyl ester was continued. The mixture was stirred at −60° C. for 1 h and at −50° C. for 5 h, resulting in a deep blue-colored solution. The mixture was slowly quenched with 2-propanol (100 mL) and allowed to warm to 20° C. overnight, while the ammonia was gradually evaporated. The remaining ammonia and part of the THF was removed in vacuum and the residue was dissolved in ice-water (600 mL). The aqueous solution was subjected to ion-exchange column chromatography (Amberlyst 36 (wet), 1000 mL). The column was eluted with deionized water (3400 mL). The acidic fractions (pH 3-5) were collected and lyophilized to give phosphoric acid mono-[6-(5,5-dimethyl-6-phosphonooxyhexyloxy)-2,2-dimethylhexyl] ester (38 g) as a pale-brown oil. This material (38 g) was dissolved in water (100 mL) and aqueous ammonium hydroxide (28%, 80 mL) was added. The solution was filtered and lyophilized to give phosphoric acid mono-[6-(5,5-dimethyl-6-phosphonooxyhexyloxy)2,2-dimethylhexyl] ester, tetra-ammonium salt (35.8 g, 86%) as an off-white solid. Mp 185-186° C. ¹H NMR (300 MHz, D₂O, HDO=4.81 ppm): δ (ppm): 3.41 (t, 4H, J=6.3 Hz), 3.34 (d, 4H, J=3.6 Hz), 1.47-1.42 (m, 4H), 1.16-1.13 (m, 8H), 0.76 (s, 12H). ¹³C NMR (75 MHz, D₂O/CD₃OD, 5% v/v, CD₃OD=49.15 ppm): 6 (ppm): 74.3 (d, J=5.3 Hz), 71.8, 39.4, 35.0 (d, J=7.4 Hz), 30.9, 24.6, 21.1. HRMS (LSIMS, gly): Calcd for C₁₆H₃₇O₉P₂ (MH⁺): 435.1912, found: 435.1947.

### Example 9

### Phosphoric Acid mono-(2,2,12,12-tetramethyl-7-oxo-13-phosphonooxytridecyl) ester, diammonium salt (Compound D)

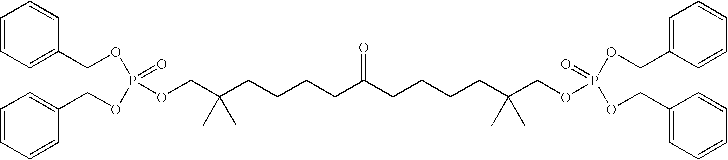

2,2,12,12-Tetramethyl-1,13-bis-(dibenzyloxyphosphoryloxy)-tridecan-7-one. Under N₂ atmosphere, a 2-L three-necked flask equipped with mechanical stirrer, thermometer, dropping funnel and N₂ inlet adapter was loaded with 1,13-dihydroxy-2,2,12,12-tetramethyltridecan-7-one (83.90 g, 292 mmol), 1,2,4-triazole (155 g, 2.24 mol), 4-dimethylaminopyridine hydrochloride (2.2 g, 13.9 mmol) and acetonitrile (500 mL). A solution of dibenzyl diisopropylphosphoramidite (376.5 g, 1.09 mmol) in acetonitrile (250 mL) was added dropwise at 25° C., resulting in a slight exothermic reaction. The mixture was stirred at 20° C. for 16 h. Water (14.2 g, 0.79 mol) was added in one portion and the solution was stirred at 20° C. for 5 h. After the addition of methylene chloride (350 mL), the reaction solution was stirred at 20° C. for 30 min and cooled to −40° C. 3-Chloroperoxybenzoic acid (77%, 245 g, 1.09 mmol.) was added in 60 portions, keeping the internal temperature below −25° C. The mixture was stirred for 16 h and allowed to warm to 20° C. The reaction mixture was poured into water (2.25 L) and methylene chloride (1.45 L). The aqueous layer was extracted with methylene chloride (500 mL). The combined organic layers were washed with 0.5 M aqueous NaS₂O₄ solution (1000 mL), saturated aqueous Na₂CO₃ solution (1000 mL), and saturated aqueous NaCl solution (2×1500 mL), dried over MgSO₄, and concentrated in vacuum to give the crude product as a colorless oil (350 g). The crude product was subjected to column chromatography on silica (3300 g, 230-400 mesh) using hexanes/ethyl acetate (3:1, 2:1 then 4:3) as eluent to afford 2,2,12,12-tetramethyl-1,13-bis-(dibenzyloxyphosphoryloxy)-tridecan-7-one (94.0 g, 40%) as a colorless oil. ¹H NMR (300 MHz, CDCl₃/TMS): δ (ppm): 7.31 (br s, 20H), 5.02 (d, 8H, J=6.4 Hz), 3.62 (d, 4H, J=4.5 Hz), 2.30 (t, 4H, J=7.5 Hz), 1.46 (m, 4H), 1.16 (m, 8H), 0.81 (s, 12H). ¹³C NMR (75 MHz, CDCl₃/TMS): δ (ppm): 210.8, 135.8 (d, J=6.3 Hz), 128.4, 127.7, 75.5 (d, J=6.4 Hz), 69.0 (d, J=5.1 Hz), 42.5, 38.1, 34.3 (d, J=7.8 Hz), 24.3, 23.7, 23.2. HRMS (gly): Calcd for C₄₅H₆₁O₉P₂ (MH⁺): 807.3786, found: 807.3774.

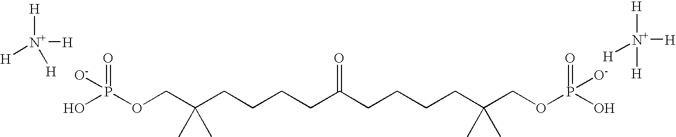

Phosphoric acid mono-(2,2,12,12-tetramethyl-7-oxo-13-phosphonooxytridecyl) ester, diammonium salt. The hydrogenation of 2,2,12,12-tetramethyl-1,13-bis-(dibenzyloxyphosphoryloxy)-tridecan-7-one was performed in two batches: Batch 1: The starting material (49.5 g, 61.4 mmol) was dissolved in 2-propanol (200 mL) in a 500-mL Parr bottle. To the solution was added 5% Pd-C catalyst (8.5 g) and the mixture was shaken at 20° C. under 50-70 psi hydrogen atmosphere on a Parr-apparatus for 26 h. The catalyst was removed by filtration through a fritted funnel and washed with methanol (50 mL). Batch 2: The starting material (45.0 g, 55.8 mmol) in 2-propanol (170 mL) was hydrogenated with 5% Pd-C (5.8 g) at 20° C. under 50-70 psi hydrogen atmosphere for 44 h. The catalyst was removed by filtration and washed with methanol (50 mL). The filtrates from both batches were combined and concentrated at a temperature below 50° C. under reduced pressure to give phosphoric acid mono-(2,2,12,12-tetramethyl-7-oxo-13-phosphonooxytridecyl) ester (66.7 g) as a colorless viscous oil. This oil was dissolved in water (200 mL) and 28-30% aqueous ammonium hydroxide solution (120 mL) was added. The formed emulsion was extracted with diethyl ether (2×200 mL) to give a clear aqueous solution. The aqueous solution was lyophilized, affording phosphoric acid mono-(2,2,12,12-tetramethyl-7-oxo-13-phosphonooxytridecyl) ester, diammonium salt (56.1 g, 98%) as a white solid. Mp 191-193° C. ¹H NMR (300 MHz, D₂O, HDO=4.80 ppm): δ (ppm): 3.43 (d, 4H, J=4.3 Hz), 2.55 (t, 4H, J=7.4 Hz), 1.52-1.49 (m, 4H), 1.22-1.20 (m, 8H), 0.84 (s, 12H). ¹³C NMR (75 MHz, D₂O/CD₃OD, 10% v/v, CD₃OD=49.10 ppm): δ (ppm): 218.3, 74.3 (d, J=5.5 Hz), 43.6, 39.5, 35.0 (d, J=8.0 Hz), 25.7, 24.6, 24.2. HRMS (ESI-FT-ICR): Calcd for C₁₇H₃₆O₉P₂Na (MNa⁺): 469.1727, found: 469.1802.

### Example 10

### Phosphoric Acid mono-(2,2,14,14-tetramethyl-8-oxo-15-phosphonooxy-pentadecyl) Ester, Diammonium Salt (Compound E)

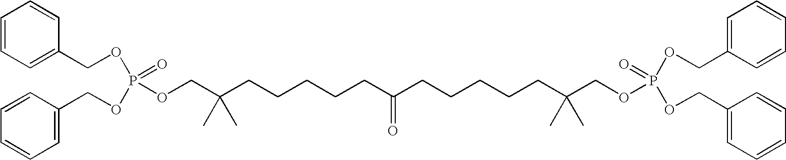

2,2,14,14-Tetramethyl-1,15-bis-(dibenzyloxyphosphoryloxy)-pentadecan-8-one. Under N₂ atmosphere, an oven-dried 2-L three-necked flask equipped with mechanical stirrer, thermometer, dropping funnel and N₂ inlet adapter was loaded with 1,15-dihydroxy-2,2,14,14-tetramethylpentadecan-8-one (86.1 g, 274 mmol), 1,2,4-triazole (155 g, 2.24 mol), 4-dimethylaminopyridine hydrochloride (2.3 g, 14.5 mmol) and acetonitrile (450 mL). A solution of dibenzyl diisopropylphosphoramidite (380 g, 1.1 mmol) in acetonitrile (450 mL) was added dropwise at 25° C. The mixture was stirred for 46 h and water (14.2 g, 0.79 mol) was added in one portion. The solution was stirred at 20° C. for 5 h. Methylene chloride (350 mL) was added and the solution was stirred for 30 min, then cooled to −40° C. 3-Chloroperoxybenzoic acid (77%, 247 g, 1.1 mol.) was added in 24 portions, keeping the temperature below −25° C. The mixture was stirred for 16 h, allowed to warm to 20° C., and poured into water (3.0 L) and methylene chloride (2.45 L). The aqueous layer was extracted with methylene chloride (300 mL). The combined organic layers were washed with aqueous 0.5 M NaS₂O₄ solution (2.0 L), saturated aqueous Na₂CO₃ solution (2.0 L), and saturated aqueous NaCl solution (2×2 L), dried over MgSO₄, and concentrated in vacuum to give the crude product as a colorless oil (384 g). The crude product was subjected to column chromatography on silica (3.7 kg, 230-400 mesh) using hexanes/ethyl acetate (4:1, 3:1, then 2:1) as eluent, affording 2,2,14,14-tetramethyl-1,15-bis-(dibenzyloxyphosphoryloxy)-pentadecan-8-one (127.4 g, 56%) as a colorless oil. ¹H NMR (300 MHz, CDCl₃/TMS): 6 (ppm): 7.36-7.29 (m, 20H), 5.06-5.02 (m, 8H), 3.66 (d, 4H, J=4.6 Hz), 2.34 (t, 4H, J=7.3 Hz), 1.55-1.51 (m, 4H), 1.20-1.19 (m, 12H), 0.84 (s, 12H). ¹³C NMR (75 MHz, CDCl₃/TMS): δ (ppm): 210.7, 135.7 (d, J=6.7 Hz), 128.3, 128.2, 127.6, 75.3 (d, J=6.6 Hz), 68.9 (d, J=5.4 Hz), 42.4, 38.0, 34.2 (d, J=7.8 Hz), 29.7, 23.5, 23.4, 23.2. HRMS (ESI): Calcd for C₄₇H₆₅O₉P₂ (MH⁺): 835.4104, found 835.4195. HPLC (Alltech Alltima C₈, 5 m, 250 mm×4.6 mm, acetonitrile/water 70/30, flow rate 1 mL/min, retention time 32.6 min, UV detection): 97.6%.

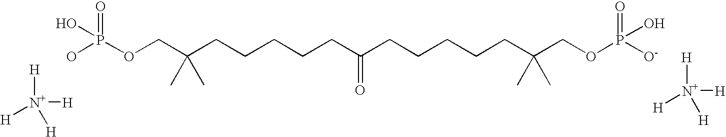

Phosphoric acid mono-(2,2,14,14-tetramethyl-8-oxo-15-phosphonooxypentadecyl) ester, diammonium salt. The hydrogenation of 2,2,14,14-tetramethyl-1,15-bis-(dibenzyloxyphosphoryloxy)-pentadecan-8-one was performed in two batches: Batch 1: The starting material (55.0 g, 66.0 mmol) was dissolved in 2-propanol (170 mL) in a 500-mL Parr bottle. To the solution was added 5% Pd-C catalyst (8.11 g) and the mixture was shaken at 20° C. under 50-70 psi hydrogen atmosphere for 26 h using a Parr-apparatus. The catalyst was removed by filtration through a fritted funnel, and washed with methanol (50 mL). Batch 2: To a solution of the starting material (50.5 g, 60.0 mmol) in 2-propanol (170 mL) was added 5% Pd-C catalyst (8.07 g) and the mixture was shaken at 20° C. under 50-70 psi hydrogen for 20 h. The catalyst was removed by filtration and washed with methanol (50 mL). The filtrates from both batches were combined and concentrated at below 50° C. under reduced pressure to give phosphoric acid mono-(2,2,14,14-tetramethyl-8-oxo-15-phosphonooxypentadecyl) ester (61.0 g, 99%) as a colorless viscous oil. This oil was dissolved in water (350 mL) and 30% aqueous ammonium hydroxide solution (130 mL) was added, causing a slightly exothermic reaction. The formed emulsion was stirred for 2 h and extracted with diethyl ether (2×100 mL) to give a clear aqueous solution. The aqueous solution was lyophilized, affording phosphoric acid mono-(2,2,14,14-tetramethyl-8-oxo-15-phosphonooxy-pentadecyl) ester, diammonium salt (59.5 g, 93%) as a white solid. Mp 187-189° C. ¹H NMR (300 MHz, D₂O, HDO=4.80 ppm): δ (ppm): 3.38 (d, 4H, J=3.8 Hz),2.47 (t, 4H, J=7.1 Hz), 1.51-1.47(m, 4H), 1.19(m, 12H),0.80(s, 12H). ¹³C NMR (75 MHz, D₂O/CD₃OD, 90/10 v/v, CD₃OD=49.15 ppm): δ (ppm): 217.7, 74.2 (d, J=5.2 Hz), 43.5, 39.6, 34.9 (d, J=7.8 Hz), 30.8, 24.8, 24.5, 24.2. HRMS (LSIMS, gly): Calcd for C₁₉H₄₁O₉P₂ (MH⁺): 475.2226, found: 475.2225. HPLC: 98.2% pure.

### 6.2. Example: Effects of an Illustrative Compound of the Pathway on Obese Female Zucker Rats

In a number of different experiments, phosphoric acid mono-[6-5,5-dimethyl-6-(phosphonooxyhexyloxy)-2,2-dimethylhexyl]ester (hereinafter, “Compound A”), an illustrative compound of the invention, or one of two reference compounds (bis(6-Hydroxy-5,5-dimethylhexyl)ether; hereinafter, “Compound 1”, or rosiglitazone maleate salt (5-{4-[2-(Methyl-pyridin-2-yl-amino)-ethoxy]-benzyl}-thiazolidine-2,4-dione)); hereinafter, “Compound 2”) were administered daily to 11-13 week old chow fed obese female Zucker rats for 14 days in the morning by oral gavage in 20% ethanol/80% polyethylene glycol-200 (dosing vehicle)(“EP”). Compounds A and 2 were administered at doses of 100 mg/kg of body weight, whereas Compound 1 was administered at a dose of 5 mg/kg body weight. The dosing vehicle was administered to control animals in parallel experiments.

Body weight was determined daily prior to dosing. Animals were allowed free access to rodent chow and water throughout the study. Blood glucose was determined after a 6-hour fast in the afternoon without anesthesia from a tail vein. Serum was also prepared from a blood sample subsequently obtained from the orbital venous plexus (with O₂/CO₂ anesthesia) prior to and after one week treatment and used lipid and insulin determinations. At two weeks, blood glucose was again determined after a 6-hour fast without anesthesia from a tail vein. Soon thereafter, animals were sacrificed by CO₂ inhalation in the afternoon and cardiac blood serum was collected and assessed for various lipids and insulin.

Generally, Compound A improved the ratio of non-HDL cholesterol to HDL cholesterol content relative to both control animals and animals treated with a reference compound. Additionally, Compound A generally reduced serum triglyceride content and did not cause the body weight increases seen in reference compound-treated animals.

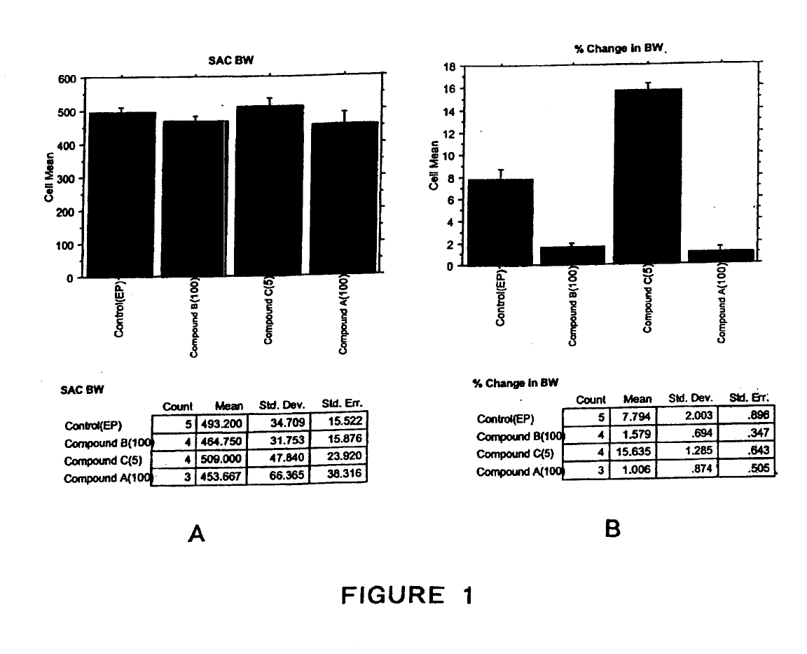

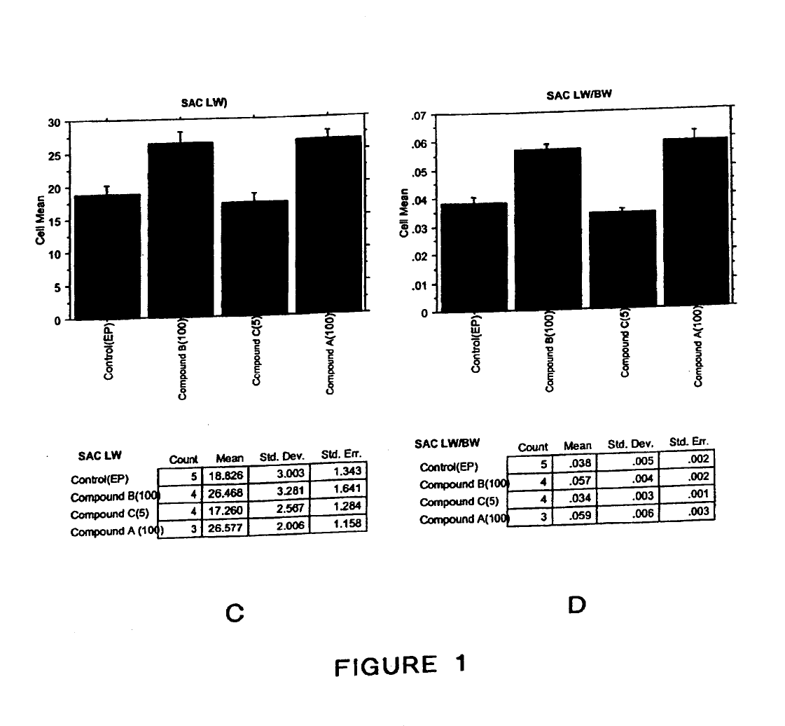

The data concerning Compound A are graphically depicted in FIGS. 1-4. Following treatment with Compound A or Compound 1, liver to body weight increased, whereas it was slightly reduced in Compound 2-treated animals. FIG. 1A shows the mean weight of the experimental animals and FIG. 1B shows the weekly percent weight gain in the Zucker rats during treatment. Control rats gained almost 8% percent of their initial weight after two weeks respectively. With Compound A treatment, the test animals gained only 1% or their initial weight, similar to the weight gain observed in Compound 1-treated animals (1.5%). In contrast, Compound 2 treatment caused increased weight gain (greater than 15%). The liver weight and the liver-to-body weight ratio were determined after two weeks of treatment at the time of sacrifice (FIGS. 1C and 1D, respectively).

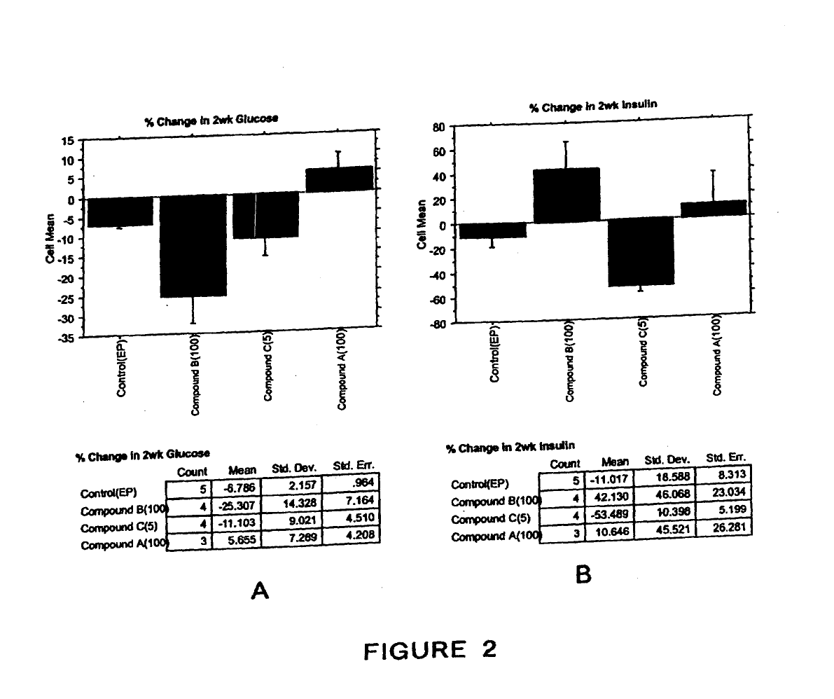

Blood glucose (FIG. 2A) and serum insulin levels (FIG. 2B) were determined from fasted rats just prior to and following treatment. Blood glucose was maintained at slightly elevated levels for 10-12 week old obese Zucker rats during treatment, whereas treatment with Compounds 1 and 2 resulted in a reduction of glucose levels. Relative to pretreatment values, serum insulin (FIG. 2B) rose slightly in Compound A-treated animals. For reference Compounds 1 and 2, serum insulin levels were increased and reduced, respectively, following two weeks of treatment.

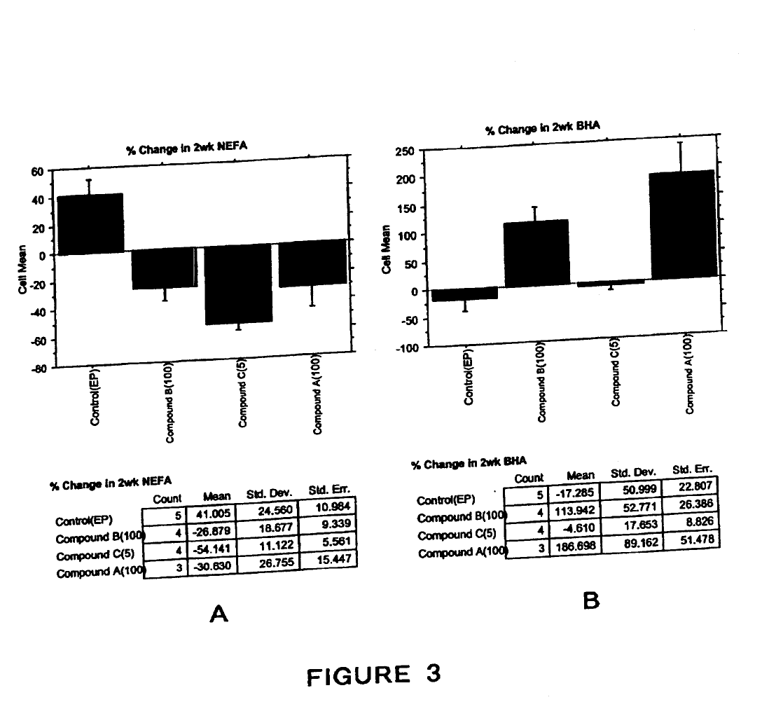

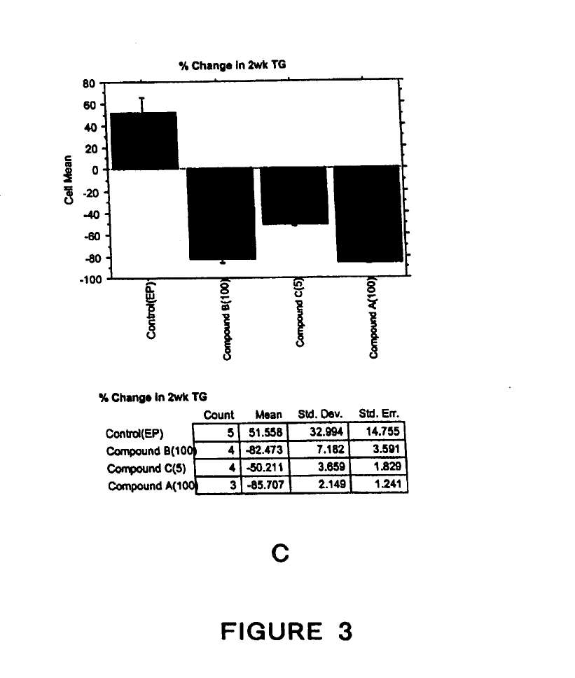

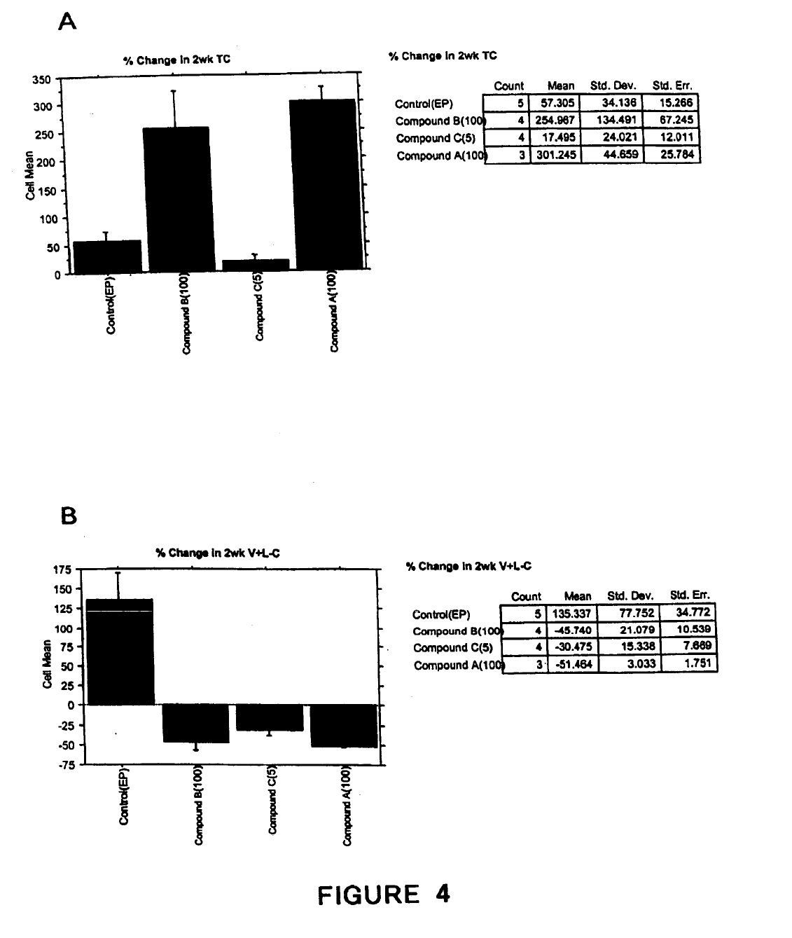

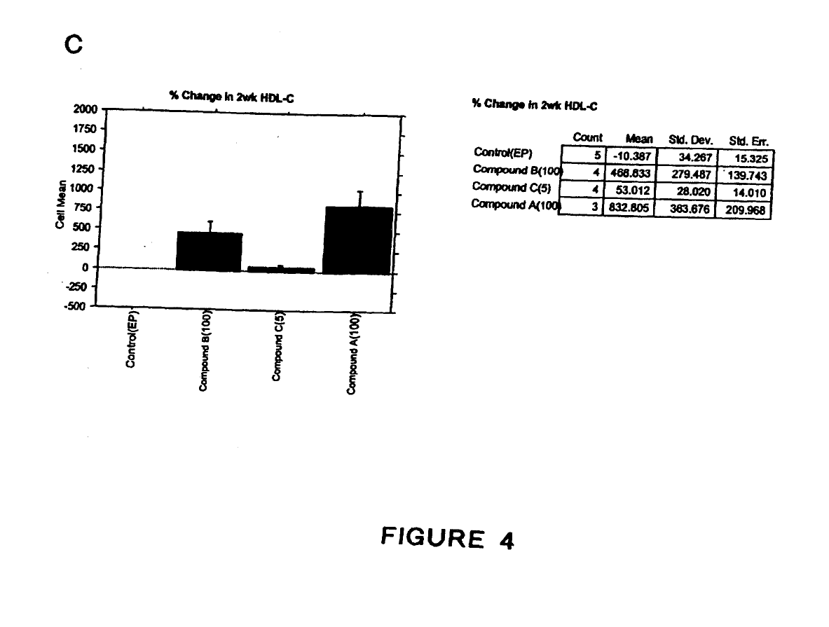

Compound A treatment reduced serum levels of harmful triglycerides (FIG. 3C), reduced serum levels of harmful non-esterified fatty acids (FIG. 3A), and elevated levels of the beneficial β-hydroxy butyrate (FIG. 3B). Compound A treatment elevated serum total cholesterol (FIG. 4A). For Compound 1, total cholesterol was only modestly elevated (FIG. 4A). Elevation in serum cholesterol observed with Compound A were largely reflected by a marked elevation in HDL-cholesterol. After a two-week treatment with Compound A, HDL-cholesterol was elevated 9-fold (FIG. 4C), a greater elevation than seen with either reference compound.

The data from FIG. 1-FIG. 4 are summarized in Table 1 below:

TABLE 1

| | | | | HDL-C/ | | | |
|---|---|---|---|---|---|---|---|
| | | Treat | mg/kg/ | nonHD | | | Non |
| Compd | (N) | days | day | L-C | TG | TC | HDL-C |
| Ctrl. | 4 | 14 | 0 | 138.3 ± 44.7 | −19.8 ± 15.5 | 48.7 ± 18.7 | 33.9 ± 10.8 |
| A | 3 | 14 | 100 | −51.5 ± 1.8 | 833.8 ± 209.9 | −85.7 ± 1.2 | −30.6 ± 15.4 |
| 1 | 4 | 14 | 100 | −45.7 ± 10.5 | 468.8 ± 139.7 | −82.5 ± 3.6 | −26.9 ± 9.3 |
| 2 | 4 | 14 | 5 | −30.5 ± 7.7 | 53 ± 14 | −50.2 ± 1.8 | −54.1 ± 5.6 |

| Compd | HDL-C | Glucose | Insulin | NEFA | BHA |
|---|---|---|---|---|---|
| Ctrl. | 3.4 ± 12.4 | −7.7 ± 0.5 | −10.4 ± 10.7 | 0.036 ± 0.002 | 7.5 ± 1.1 |
| A | 186.7 ± 51.5 | 5.6 ± 4.2 | 10.6 ± 26.3 | 0.059 ± 0.003 | 1 ± 0.5 |
| 1 | 113.9 ± 26.4 | −25.3 ± 7.2 | 42.1 ± 23 | 0.056 ± 0.002 | 1.6 ± 0.3 |
| 2 | −4.6 ± 8.8 | −11.1 ± 4.5 | −53.3 ± 5.2 | 0.034 ± 0.001 | 15.6 ± 0.6 |

Additional therapeutic effects of illustrative compounds of the invention are displayed in Table 2.

Accordingly, the compounds of the present invention, as illustrated by Compound A and the ones mentioned in Table 1, or pharmaceutically acceptable salts thereof, are useful for improving the ratio of HDL:non-HDL cholesterol in the blood, reducing serum triglycerides, and/or elevating HDL-cholesterol, without the adverse side effect of promoting weight gain in a patient to whom the compound is administered.

TABLE 2
Examples of effects of oral daily treatment of obese female Zucker rats with
compounds of the invention for fourteen days (n is number of animals per experiment)

| | | | | | | Percent Change from Pre-treatment | | | | | | | |
|---|---|---|---|---|---|---|---|---|---|---|---|---|---|
| | | | Dose | % | | | | | | | | | |
| | | | (mg/kg/ | wt. | HDL-C/ | | | Non | | | | | |
| Compd | Expt. # | n | day) | gain | nonHDL-C | TG | TC | HDL-C | HDL-C | Glucose | Insulin | NEFA | BHA |
| Vehicle | LR92 | 4 | — | 7 | 2 | 1 | −3 | 24 | −10 | −5 | −9 | 11 | 62 |
| A | LR92 | 4 | 10 | 9 | 2 | 11 | 115 | 67 | 179 | −9 | −7 | 14 | 102 |
| A | LR92 | 4 | 30 | 3 | 4 | −31 | 164 | 71 | 228 | 1 | −16 | −32 | 141 |
| A | LR92 | 4 | 100 | 4 | 8 | — | 287 | 11 | 559 | 6 | 0 | −53 | 257 |
| Vehicle | LR99 | 5 | — | 8 | 2 | 19 | 1 | 56 | −19 | −7 | 22 | −3 | 42 |
| D | LR99 | 5 | 10 | 12 | 2 | 11 | 8 | 25 | 5 | −5 | 4 | −13 | 123 |
| D | LR99 | 5 | 30 | 9 | 2 | −12 | 21 | 22 | 28 | −4 | 2 | −25 | 271 |
| D | LR99 | 5 | 100 | 8 | 2 | −1 | 55 | 71 | 57 | −3 | 14 | −22 | 814 |
| Vehicle | LR105 | 4 | — | 6 | 1 | 29 | 3 | 50 | −17 | −4 | −25 | 23 | 46 |
| B | LR105 | 4 | 10 | 7 | 2 | 31 | −4 | 15 | −10 | 12 | −21 | 18 | 175 |
| B | LR105 | 4 | 30 | 6 | 2 | 4 | 66 | 42 | 91 | 1 | −17 | −7 | 202 |
| B | LR105 | 4 | 100 | 6 | 3 | 1 | 150 | 105 | 408 | 2 | 28 | −24 | 171 |
| Vehicle | LR107 | 4 | — | 8 | 8 | 3 | −4 | 3 | 97 | −14 | −11 | −13 | 139 |
| E | LR107 | 4 | 10 | 11 | 6 | 39 | 3 | 6 | 129 | −6 | 25 | −5 | 140 |
| E | LR107 | 4 | 30 | 10 | 4 | −2 | 126 | 51 | 174 | −1 | −12 | −28 | 73 |
| E | LR107 | 4 | 100 | 4 | 8 | −51 | 161 | 20 | 246 | −4 | −15 | −40 | 134 |
| Vehicle | LR65 | 4 | — | 11 | 2 | 19 | 2 | 76 | −18 | −7 | 2 | −16 | 107 |
| M | LR65 | 3 | 100 | 20 | 2 | −4 | 70 | 84 | 65 | 2 | 26 | −53 | 145 |
| Vehicle | LR28 | 5 | — | 1 | 1 | −41 | −14 | −39 | 58 | −16 | −43 | −37 | 236 |
| L | LR28 | 2 | 80 | 0 | 2 | −13 | −4 | −17 | 8 | 9 | 21 | 62 | 575 |
| Vehicle | LR56 | 5 | — | 13 | 2 | 26 | 1 | 43 | −17 | 5 | −21 | 7 | 56 |
| K | LR56 | 3 | 100 | 11 | 3 | 7 | 12 | −5 | 23 | −21 | 0 | 43 | 103 |
| Vehicle | LR52 | 4 | — | 8 | 2 | −6 | −14 | −16 | −7 | 3 | −36 | −7 | 31 |
| J | LR52 | 4 | 30 | 11 | 2 | −10 | 9 | −7 | 20 | 18 | −41 | −38 | 21 |
| Vehicle | LR22 | 3 | — | 14 | 2 | −7 | 18 | 47 | 3 | 6 | −46 | −34 | 35 |
| H | LR22 | 2 | 53 | 16 | 2 | −32 | 5 | −10 | 16 | −23 | −49 | −55 | 48 |
| Vehicle | LR65 | 4 | — | 11 | 2 | 19 | 2 | 76 | −18 | −7 | 2 | −16 | 107 |
| C | LR65 | 5 | 30 | 13 | 2 | 22 | 6 | 52 | −19 | 9 | 11 | −33 | 123 |

### 6.3. Example: Effect of an Illustrative Compound of the Invention on the Synthesis of Total Lipids in Hepatocytes Isolated from a Male Sprague-Dawley Rat

A male Sprague-Dawley rate was anesthetized by administration of sodium pentobarbitol by intraperitoneal at 80 mg/kg. In situ perfusion of the liver was performed as follows. The abdomen of the animal was opened, the portal vein canulated, and the liver perfused with WOSH solution (149 mM NaCl, 9.2 mM Na HEPES, 1.7 mM Fructose, 0.5 mM EGTA, 0.029 mM Phenol red, 10 U/ml heparin, pH 7.5) at a flow rate of 30 ml/min for 6 minutes. To digest the liver, DSC solution (6.7 mM KCl, 143 mM NaCl, 9.2 mM Na HEPES, 5 mM CaCl₂-2H₂O, 1.7 mM Fructose, 0.029 mM Phenol red, 0.2% BSA, 100 U/ml collagenase Type 1,160 BAEE/ml trypsin inhibitor, pH 7.5) was perfused through the liver at a flow rate of 30 ml/min for 6 minutes at a temperature of 37° C. After digestion, cells were dispersed in a solution of DMEM-(DMEM containing 2 mM GlutMax-1, 0.2% BSA, 5% FBS, 12 nM insulin, 1.2 μM hydrocortisone) to stop the digestion process. The crude cell suspension was filtered through three layers of stainless steel mesh with pore sizes of 250, 106, and 75 μm respectively. Filtered cells were centrifuged at 50×g for two minutes and the supernatant discarded. The resulting cell pellet was resuspended in DMEM and centrifuged again. This final cell pellet was resuspended in DMEM+HS solution (DMEM containing 2 mM GlutMax-1, 20 mM delta-aminolevulinic acid, 17.4 mM MEM nonessential amino acids, 20% FBS, 12 nM insulin, 1.2 μM hydrocortisone) and plated to form monolayer cultures at a density of 100×10³ cells/cm² on collagen coated culture dishes. Four hours after initial plating, media was changed to DMEM+(DMEM containing 2 mM GlutMax-1, 20 nM delta-aminolevulinic acid, 17.4 mM MEM non-essential amino acids, 10% FBS, 12 nM insulin, 1.2 μM hydrocortisone) and remained on cells overnight.

To test the effect of an illustrative compound of the invention on synthesis rates of total lipids, the monolayer cultures were exposed to 1, 3, 10, 30, 100, or 300 μM of Compound A or 10 μM of Compound 1 in DMEM+containing 1 μCi/ml 14C-acetate, D-glucose, hepes, glutamine, leucine, alanine, lactate, pyruvate, non-essential amino acids, BSA, and gentamicine. Control cells were exposed to the same media lacking lovastatin or the test compounds. All cells were exposed to 0.1% DMSO. Metabolic labeling with ¹⁴C-acetate continued for 4 hr at 37° C. After labeling, cells were washed twice with 1 mL of PBS followed by addition of scintallant (Microscent E) and counted on Topcount®.

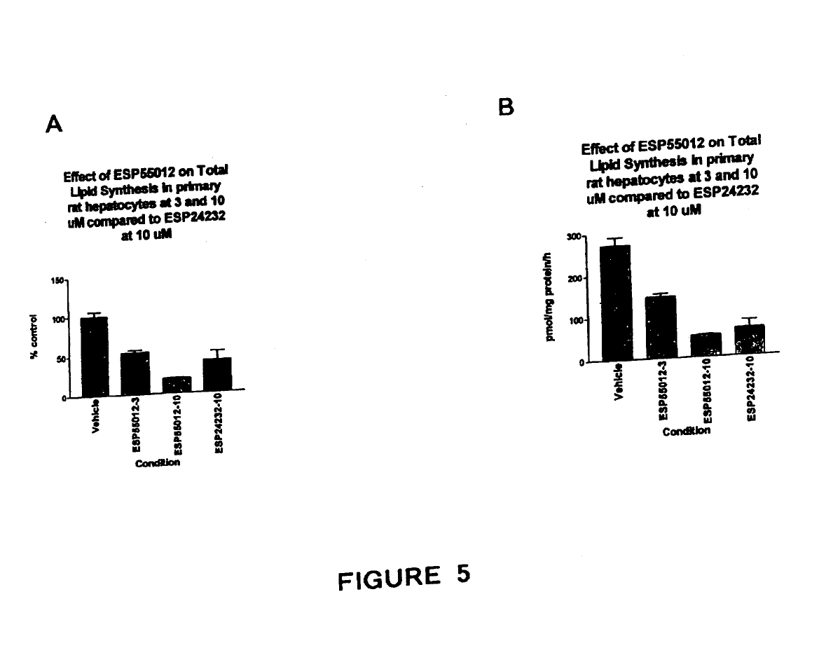

FIG. 5A shows the rates of total lipid synthesis following treatment with Compounds A and B. Data are represented as a percent of no compound treatment (Vehicle control). Data are represented as the mean of three measurements +/−one standard deviation. The data indicate that the illustrative compound of the invention is useful for inhibition of lipid synthesis. In particular, Compound A at 3 and 10 μM reduced the rates of total lipid synthesis by at least 97% in the rat hepatocyte cells. Compound 1 also reduced the rates of total lipids by at least 65% in the rat hepatocyte cells.

FIG. 5B shows the lipid to protein synthesis ratio in primary rat hepatocytes following treatment with Compounds A and 1. Data are represented as the ratio of hourly production of pmol lipid:mg protein. Data are represented as the mean of three measurements +/−one standard deviation. The data confirm the findings in FIG. 5A that the illustrative compound of the invention are useful for inhibition of lipid synthesis.

Table 3 presents IC₅₀ values indicating inhibition of lipid synthesis in primary hepatocytes for the compounds of this invention.

TABLE 2
Examples of IC₅₀

| Compound | IC₅₀ (μm) |
|---|---|
| 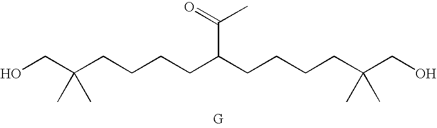 | 62 |
| 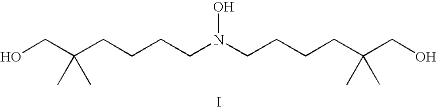 | 64 |
| 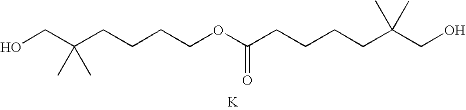 | 14 |
| 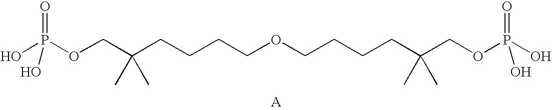 | 43 |
| 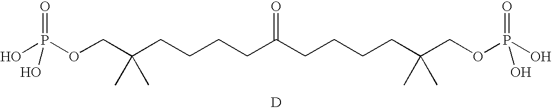 | 16 |
| 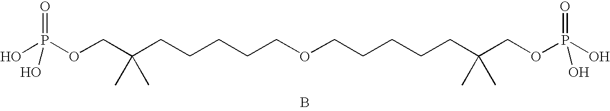 | 28 |
| 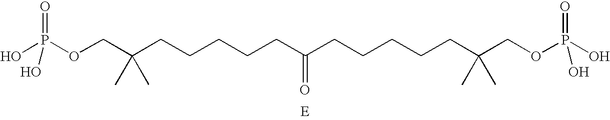 | 35 |

Accordingly, the compounds of the present invention, in which Compound A or a pharmaceutically acceptable salt thereof is illustrative, are useful for reducing lipid synthesis in a patient.

The present invention is not to be limited in scope by the specific embodiments disclosed in the examples which are intended as illustrations of a few aspects of the invention and any embodiments which are functionally equivalent are within the scope of this invention. Indeed, various modifications of the invention in addition to those shown and described herein will become apparent to those skilled in the art and are intended to fall within the appended claims.

What is claimed is:

1. A compound of formula I: 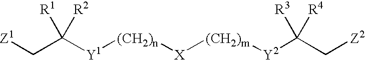
and pharmaceutically acceptable salts, solvates, hydrates, or prodrugs thereof, wherein:
Z¹ and Z² are independently —OH, —OPO₃H, —OP₂O₆H₂, —OPO₃-(nucleotide), —OP₂O₆(H)-(nucleotide);
R¹ and R³ are independently hydrogen, methyl, or phenyl;
R² and R⁴ are independently methyl or phenyl;
m and n are independently 0, 1, 2, 3, 4, 5, or 6;
Y¹ and Y² are independently —CH₂, 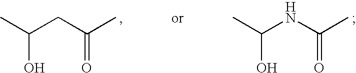
and
X is O, S, Se, C(O), C(H)F, CF₂, S(O), NH, O—P(O)(OH)—O, NH—C(O)—NH or NH—C(S)—NH,
with the proviso that when:
X is O and n and m are 3;
or X is C(O), S, or S(O) and n and m are 1-4;
Y¹ and Y² are —CH₂—; and
R¹-R⁴ are independently methyl or phenyl,
then at least one of Z¹ and Z² is —OPO₃-(nucleotide) or —OP₂O₆(H)-(nucleotide).

2. A compound of formula I: 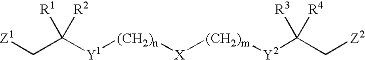
or pharmaceutically acceptable salts, solvates, hydrates, or prodrugs thereof, wherein:
Z¹ and Z² are independently —OH, —OPO3H, —OP₂O₆H₂, —OPO₃-(nucleotide), —OP₂O₆(H)-(nucleotide);
R¹ and R² are taken together to form a cycloalkyl ring of 3 to 6 carbons;
R³ is hydrogen, methyl, or phenyl;
R⁴ is methyl or phenyl;
m and n are independently 0, 1, 2, 3, 4, 5, or 6;
Y¹ and Y² are independently —CH₂, 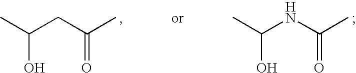
and
X is O, S, Se, C(O), C(H)F, CF₂, S(O), NH, O—P(O)(OH)—O, NH—C(O)—NH or NH—C(S)—NH,
with the proviso that when:
X is O and n and m are 3;
or X is C(O), S, or S(O) and n and m are 1-4;
Y¹ and Y² are —CH₂—;
R¹ and R² are taken together to form a cycloalkyl ring of 3 to 6 carbons; and R³ and R⁴ are independently methyl or phenyl,
then at least one of Z¹ and Z² is —OPO₃-(nucleotide) or —OP₂O₆(H)-(nucleotide).

3. A compound of formula I: 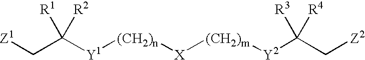
or pharmaceutically acceptable salts, solvates, hydrates, or prodrugs thereof, wherein:
Z¹ and Z² are independently —OH, —OPO₃H, —OP₂O₆H₂, —OPO₃-(nucleotide), —OP₂O₆(H)-(nucleotide);
R¹ and R² are taken together to form a cycloalkyl ring of 3 to 6 carbons;
R³ and R⁴ are taken together to form a cycloalkyl ring of 3 to 6 carbons;
m and n are independently 0, 1, 2, 3, 4, 5, or 6;
Y¹ and Y² are independently —CH₂, 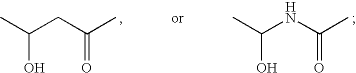
and
X is O, S, Se, C(O), C(H)F, CF₂, S(O), NH, O—P(O)(OH)—O, NH—C(O)—NH or NH—C(S)—NH,
with the proviso that when:
X is O and n and m are 3;
or X is C(O), S, or S(O) and n and m are 1-4;
Y¹ and Y² are —CH₂—;
R¹ and R² are taken together to form a cycloalkyl ring of 3 to 6 carbons; and R³ and R⁴ are taken together to form a cycloalkyl ring of 3 to 6 carbons,
then at least one of Z¹ and Z² is —OPO₃-(nucleotide) or —OP₂O₆(H)-(nucleotide).

4. A composition comprising a compound of claim 1 and a pharmaceutically acceptable vehicle, excipient, or diluent.

5. A composition comprising a compound of claim 2 and a pharmaceutically acceptable vehicle, excipient, or diluent.

6. A composition comprising a compound of claim 3 and a pharmaceutically acceptable vehicle, excipient, or diluent.

7. A method for treating or preventing cardiovascular disease, dyslipidemia, dyslipoproteinemia, a disorder of glucose metabolism, hypertension, or impotence in a patient, comprising administering to a patient in need of such treatment or prevention a therapeutically or prophylactically effective amount of a compound of claim 1.

8. A method for treating or preventing Alzheimer's Disease, Syndrome X, a peroxisome proliferator activated receptor-associated disorder, septicemia, a thrombotic disorder, obesity, pancreatitis, renal disease, cancer, inflammation, or bacterial infection in a patient, comprising administering to a patient in need of such treatment or prevention a therapeutically or prophylactically effective amount of a compound of claim 1.

9. A method for treating or preventing cardiovascular disease, dyslipidemia, dyslipoproteinemia, a disorder of glucose metabolism, hypertension, or impotence in a patient, comprising administering to a patient in need of such treatment or prevention a therapeutically or prophylactically effective amount of a compound of claim 2.

10. A method for treating or preventing Alzheimer's Disease, Syndrome X, a peroxisome proliferator activated receptor-associated disorder, septicemia, a thrombotic disorder, obesity, pancreatitis, renal disease, cancer, inflammation, or bacterial infection in a patient, comprising administering to a patient in need of such treatment or prevention a therapeutically or prophylactically effective amount of a compound of claim 2.

11. A method for treating or preventing cardiovascular disease, dyslipidemia, dyslipoproteinemia, a disorder of glucose metabolism, hypertension, or impotence in a patient, comprising administering to a patient in need of such treatment or prevention a therapeutically or prophylactically effective amount of a compound of claim 3.

12. A method for treating or preventing Alzheimer's Disease, Syndrome X, a peroxisome proliferator activated receptor-associated disorder, septicemia, a thrombotic disorder, obesity, pancreatitis, renal disease, cancer, inflammation, or bacterial infection in a patient, comprising administering to a patient in need of such treatment or prevention a therapeutically or prophylactically effective amount of a compound of claim 3.

13. A method for treating or preventing a cardiovascular disease in a patient, comprising administering to a patient in need of such treatment or prevention a therapeutically or prophylactically effective amount of a compound of claim 1.

14. A method for treating or preventing a cardiovascular disease in a patient, comprising administering to a patient in need of such treatment or prevention a therapeutically or prophylactically effective amount of a compound of claim 2.

15. A method for treating or preventing a cardiovascular disease in a patient, comprising administering to a patient in need of such treatment or prevention a therapeutically or prophylactically effective amount of a compound of claim 3.

16. A method for treating or preventing a dyslipidemia in a patient, comprising administering to a patient in need thereof a therapeutically or prophylactically effective amount of a compound of claim 1.

17. A method for treating or preventing a dyslipidemia in a patient, comprising administering to a patient in need thereof a therapeutically or prophylactically effective amount of a compound of claim 2.

18. A method for treating or preventing a dyslipidemia in a patient, comprising administering to a patient in need thereof a therapeutically or prophylactically effective amount of a compound of claim 3.

19. A method for treating or preventing hypertension in a patient, comprising administering to a patient in need thereof a therapeutically or prophylactically effective amount of a compound of claim 1.

20. A method for treating or preventing hypertension in a patient, comprising administering to a patient in need thereof a therapeutically or prophylactically effective amount of a compound of claim 2.

21. A method for treating or preventing hypertension in a patient, comprising administering to a patient in need thereof a therapeutically or prophylactically effective amount of a compound of claim 3.

22. A method for treating or preventing cancer in a patient, comprising administering to a patient in need thereof a therapeutically or prophylactically effective amount of a compound claim 1.

23. A method for treating or preventing cancer in a patient, comprising administering to a patient in need thereof a therapeutically or prophylactically effective amount of a compound claim 2.

24. A method for treating or preventing cancer in a patient, comprising administering to a patient in need thereof a therapeutically or prophylactically effective amount of a compound claim 3.

25. A method for treating or preventing inflammation in a patient, comprising administering to a patient in need thereof a therapeutically or prophylactically effective amount of a compound of claim 1.

26. A method for treating or preventing inflammation in a patient, comprising administering to a patient in need thereof a therapeutically or prophylactically effective amount of a compound of claim 2.

27. A method for treating or preventing inflammation in a patient, comprising administering to a patient in need thereof a therapeutically or prophylactically effective amount of a compound of claim 3.

28. A method for treating or preventing impotence in a patient, comprising administering to a patient in need thereof a therapeutically or prophylactically effective amount of a compound of claim 1.

29. A method for treating or preventing impotence in a patient, comprising administering to a patient in need thereof a therapeutically or prophylactically effective amount of a compound of claim 2.

30. A method for treating or preventing impotence in a patient, comprising administering to a patient in need thereof a therapeutically or prophylactically effective amount of a compound of claim 3.

31. A single unit dosage form comprising a compound of claim 1 in an amount from about 0.001 mg to about 200 mg.

32. The dosage form of claim 31, wherein the amount is from about 0.025 mg to about 150 mg.

33. The dosage form of claim 32, wherein the amount is from about 0.05 mg to about 100 mg.

34. The dosage form of claim 31 which is formulated for oral administration.

35. The dosage form of claim 34 which is a solid.

36. The dosage form of claim 31 which is formulated for parenteral administration.

37. The dosage form of claim 36, wherein the dosage form is a sterile solution.

38. The dosage form of claim 31, wherein the dosage form is suitable for mucosal or transdermal administration.

39. A single unit dosage form comprising a compound of claim 2 in an amount from about 0.001 mg to about 200 mg.

40. The dosage form of claim 39, wherein the amount is from about 0.025 mg to about 150 mg.

41. The dosage form of claim 39, wherein the amount is from about 0.05 mg to about 100 mg.

42. The dosage form of claim 39, which is formulated for oral administration.

43. The dosage form of claim 42 which is a solid.

44. The dosage form of claim 39, which is formulated for parenteral administration.

45. The dosage form of claim 44, wherein the dosage form is a sterile solution.

46. The dosage form of claim 39, wherein the dosage form is suitable for mucosal or transdermal administration.

47. A single unit dosage form comprising a compound of claim 3 in an amount from about 0.001 mg to about 200 mg.

48. The dosage form of claim 47, wherein the amount is from about 0.025 mg to about 150 mg.

49. The dosage form of claim 47, wherein the amount is from about 0.05 mg to about 100 mg.

50. The dosage form of claim 47, which is formulated for oral administration.

51. The dosage form of claim 50, which is a solid.

52. The dosage form of claim 47, which is formulated for parenteral administration.

53. The dosage form of claim 52, wherein the dosage form is a sterile solution.

54. The dosage form of claim 47, wherein the dosage form is suitable for mucosal or transdermal administration.

55. A method for identifying a compound useful for treating or preventing a condition in a patient comprising:
a) docking a three-dimensional structure of a test compound with a three-dimensional structure of a substrate binding site of a short-chain acyl-coenzyme A ligase and determining a first binding energy value therefor;
b) docking the three-dimensional structure of the test compound with a three-dimensional structure of a substrate binding site of a long-chain acyl-coenzyme A ligase and determining a second binding energy value therefor; and
c) determining whether the ratio of the first binding energy value and the second binding energy value.

56. The method of claim 55, wherein the short-chain acyl-coenzyme A ligase is a short chain acyl coenzyme A synthetase or butyrate-CoA ligase.

57. The method of claim 55, wherein the long-chain acyl-coenzyme A ligase is selected from the group consisting of fatty acyl CoA synthetase and palymitoyl CoA synthetase.

58. The method of claim 55, wherein the ratio is at least 2.

59. The method of claim 55, wherein the ratio is at least 10.

60. The method of claim 55, wherein the ratio is at least 100.

61. The method of claim 7, wherein the amount of compound of claim 1 is from about 0.001 mg to about 200 mg per kilogram body weigh.

62. The method of claim 8, wherein the amount of compound of claim 1 is from about 0.001 mg to about 200 mg per kilogram body weigh.

63. The method of claim 9, wherein the amount of compound of claim 1 is from about 0.001 mg to about 200 mg per kilogram body weigh.

64. The method of claim 10, wherein the amount of compound of claim 1 is from about 0.001 mg to about 200 mg per kilogram body weigh.

65. The method of claim 11, wherein the amount of compound of claim 1 is from about 0.001 mg to about 200 mg per kilogram body weigh.

66. The method of claim 12, wherein the amount of compound of claim 1 is from about 0.001 mg to about 200 mg per kilogram body weigh.
